(19) United States
(12) Patent Application Publication
(10) Pub. No.: US 2024/0316217 A1
(43) Pub. Date: Sep. 26, 2024

(54) METHODS FOR PREVENTING CARDIAC OR SKELETAL DEFECTS IN DISEASES INCLUDING MUCOPOLYSACCHARIDOSES

(71) Applicants: Regents of the University of Minnesota, Minneapolis, MN (US); REGENXBIO Inc., Rockville, MD (US)

(72) Inventors: R. Scott McIvor, St. Louis Park, MN (US); Nicholas Alexander Piers Sascha Buss, Rockville, MD (US); Troy C. Lund, Eden Prairie, MN (US); Elizabeth Braunlin, Roseville, MN (US); Lalitha R. Belur, St. Paul, MN (US); Carolyn Fairbanks, St. Paul, MN (US); Marie-Laure Nevoret, Virginia Beach, VA (US)

(21) Appl. No.: 18/264,364

(22) PCT Filed: Feb. 4, 2022
(86) PCT No.: PCT/US2022/015294

§ 371 Date: Aug. 4, 2023

Related U.S. Application Data
(60) Provisional application No. 63/146,383, filed on Feb. 5, 2021.
(60) Provisional application No. 63/185,743, filed on May 7, 2021.
(60) Provisional application No. 63/186,491, filed on May 10, 2021.
(60) Provisional application No. 63/283,920, filed on Nov. 29, 2021.

(51) Int. Cl. A61K 48/00; A61K 9/00; A61K 38/47; A61P 3/06; A61P 9/00; A61P 19/00; C12N 15/86

(52) U.S. Cl. CPC A61K 48/005; A61K 9/0019; A61K 9/0085; A61K 38/47; A61K 48/0075; A61K 48/0083; A61P 3/06; A61P 9/00; A61P 19/00; C12N 15/86; C12Y 302/01076; C12N 2750/14143

(57) ABSTRACT

A method to prevent, inhibit the progression of, reduce the severity of, or treat cardiac, vascular or skeletal dysfunction or defect(s) in a human having lysosomal storage disorder, is provided.

### CROSS-REFERENCE TO RELATED APPLICATIONS

This application is a U.S. National Stage Filing under 35 U.S.C. § 371 of International Patent Application Serial No. PCT/US2022/015294, filed Feb. 4, 2022, and published as WO 2022/170082 A1 on Aug. 11, 2022, which claims the benefit of the filing date of U.S. application No. 63/283,920, filed on Nov. 29, 2021, U.S. application No. 63/186,491, filed on May 10, 2021, U.S. application No. 63/185,743, filed on May 7, 2021, and U.S. application No. 63/146,383, filed on Feb. 5, 2021, the disclosures of which are incorporated by reference herein.

### INCORPORATION BY REFERENCE OF SEQUENCE LISTING PROVIDED AS A TEXT FILE

A Sequence Listing is provided herewith as a text file, “600A81SEQList.txt”, created on Feb. 14, 2024 and having a size of 1,678 bytes. The contents of the text file are incorporated by reference herein in their entirety.

### BACKGROUND

The mucopolysaccharidoses (MPSs) are a group of storage diseases caused by disruptions in glycosaminoglycan (GAG) catabolism, leading to their accumulation in lysosomes (Muenzer, 2004; Munoz-Rojas et al., 2008). Mucopolysaccharidosis type I (MPS 1) is caused by deficiency of the lysosomal enzyme alpha-L-iduronidase (IDUA) and Mucopolysaccharidosis type II is caused by a deficiency of lysosomal enzyme Iduronate-2-sulfatase (IDS). Current treatments for MPS diseases such as MPS I and MPSII include enzyme replacement therapy (ERT) and allogeneic hematopoietic stem cell transplantation (HSCT). ERT has shown alleviation of peripheral metabolic storage disease, and HSCT has been shown to impede neurocognitive decline in severe MPS I (Hurler syndrome), but skeletal and cardiac manifestations of MPS I and MPS II are not remedied by either of these therapies. Moreover, there is currently no therapy that effectively addresses the cardiac and skeletal manifestations in MPS disease and lysosomal storage diseases in general, e.g., see Broomfield et al. 2019; Lum et al., 2017: Stevenson et al., 2013; and Taylor et al., 2019.

### SUMMARY

The disclosure involves methods to prevent, inhibit (delay) progression of, reduce the severity of, and/or treat cardiac, vascular and/or skeletal dysfunction or defect(s) in a mammal having a lysosomal storage disorder (LSD), such as a mucopolysaccharidosis (MPS) disease. The disclosure is based, in part, on the inventors' discovery that administration of an AAV9-IDUA vector, via two different routes (e.g., IV and IT), surprisingly resulted in high levels of enzyme activity in major organs, and prevented cardiac valve dysfunction, aortic skeletal dysplasia, and neurocognitive deficit in MPS I mice (see Examples A, B and C herein), e.g., using a low IV dose. These MPSI mice are a model for genetic therapy of human MPS I and other LSDs with cardiac and skeletal impairments, such as MPS II. It was also surprising that specific regimens disclosed herein involving administration of a recombinant AAV vector, administered either intravenously (IV) or intrathecally (IT), showed high levels of enzyme activity in major organs, and prevented cardiac valve dysfunction, aortic dilation, skeletal dysplasia, and neurocognitive deficit in MPS I mice (see Examples A, B and C herein).

These results are surprising because currently approved therapies for LSDs such as ERT and HSCT, while alleviating some disease symptoms, are not curative for cardiac and skeletal defects which are the major causes of death in many LSD patients, i.e., cardiovascular failure and airway obstruction, e.g., restrictive lung disease. AAV-mediated gene therapy, administered intravenously (IV), intrathecally (IT) or via the combined routes (IV and IT), e.g., using the regimens disclosed herein, can significantly improve patient outcomes by altering cardiac and/or skeletal dysfunction in LSDs, and in particular for MPS disease, such as MPS I and MPS II. In one embodiment, a method is provided to prevent, inhibit, reduce, or treat cardiac, vascular or skeletal dysfunction in a mammal having a lysosomal storage disease. In one embodiment, a method is provided to prevent, inhibit, reduce, or treat cardiac, vascular or skeletal dysfunction in a mammal having mucopolysaccharoidosis disease using an rAAV vector (e.g., rAAV9) to deliver: the Alpha-L-iduronidase (IDUA) transgene for MPSI; the Iduronate-2-sulfatase (IDS) transgene for MPSII; the Heparan-N-sulfatase (SGSH) transgene for MPSIIIA, the N-acetylglucosaminidase (NAGLU) transgene for MPSIIIB; the Acetyl CoA glucosamine N-acetyltransferase (HGSNAT) transgene for MPSIIIC; the N-acetyl-glucosamine-6-sulfatase (GNS) transgene for MPSIIID; the N-acetylgalactosamine-6-sulfate sulfatase (GALNS) transgene for MPSIVA; the β-galactosidase (GLB1) transgene for MPSIVB; the Arylsulfatase B (ARSB) transgene for MPSVI; the β-glucuronidase (GUSB) transgene for MPS VII; or the Hyaluronidase (HYAL1) transgene for MPS IX.

The methods include administering to a mammal (e.g., a human) a first composition comprising an effective amount of a first recombinant adeno-associated virus (rAAV) vector (e.g., rAAV9) comprising an open reading frame encoding a first gene product (e.g., IDUA or IDS) and optionally a second composition comprising an effective amount of a second rAAV (e.g., rAAV9) vector comprising an open reading frame also encoding the first gene product, or encoding a second gene product, wherein if the first and second compositions are administered, they are administered doses via different routes (e.g., IV and IT/IC). In one embodiment, the first and the second gene products are the same. In one embodiment, one of the routes is intravenous (IV) administration. In one embodiment, one of the routes is intrathecal (IT) or intracisternal (IC) administration. In one embodiment, the first and second compositions are concurrently administered. In one embodiment, the first and second compositions are administered one day apart. In one embodiment, the first and second compositions are administered two days apart. In one embodiment, the first composition is administered before the second composition. In one embodiment, the first composition is administered after the second composition. In one embodiment, the first composition is intrathecally administered and the second composition is intravenously administered.

The methods also include administering to a mammal (e.g., a human) a composition comprising an effective amount of a recombinant adeno-associated virus (rAAV) vector (e.g., rAAV9) comprising an open reading frame encoding a gene product (e.g., IDUA or IDS), wherein the composition is administered via one route of administration (e.g., IV or IT/IC). In one embodiment, the route of administration is intravenous (IV) administration. In one embodiment, the route of administration is intrathecal (IT) or intracisternal (IC) administration.

In one embodiment, the method includes administering to a human with MPSI a first composition and optionally a second composition, comprising a rAAV encoding alpha-L-iduronidase (IDUA).

In one embodiment, the method includes administering to a human with MPSII a first composition and optionally a second composition, comprising a rAAV encoding iduronate-2-sulfatase (IDS).

In one embodiment, the method includes administering to a human with MPSIIIA a first composition and optionally a second composition, comprising a rAAV encoding heparan-N-sulfatase (SGSH).

In one embodiment, the method includes administering to a human with MPSIIIB a first composition and optionally a second composition, comprising a rAAV encoding N-acetylglucosaminidase (NAGLU).

In one embodiment, the method includes administering to a human with MPSIIIC a first composition and optionally a second composition, comprising a rAAV encoding acetyl CoA glucosamine N-acetyltransferase (HGSNAT).

In one embodiment, the method includes administering to a human with MPSIIID a first composition and optionally a second composition, comprising a rAAV encoding N-acetyl-glucosamine-6-sulfatase (GNS).

In one embodiment, the method includes administering to a human with MPSIVA a first composition and optionally a second composition, comprising a rAAV encoding N-acetylgalactosamine-6-sulfate sulfatase (GALNS).

In one embodiment, the method includes administering to a human with MPSIVB a first composition and optionally a second composition, comprising a rAAV encoding β-galactosidase.

In one embodiment, the method includes administering to a human with MPSVI a first composition and optionally a second composition, comprising a rAAV encoding arylsulfatase B (ARSB).

In one embodiment, the method includes administering to a human with MPSVII a first composition and optionally a second composition, comprising a rAAV encoding β-glucuronidase (GUSB).

In one embodiment, the method includes administering to a human with MPSIX a first composition and optionally a second composition, comprising a rAAV encoding hyaluronidase (HYAL1).

In one embodiment, the method includes administering to a human with Pompe disease a first composition and optionally a second composition, comprising a rAAV encoding alpha-glycosidase.

In one embodiment, the method includes administering to a human with Danon disease a first composition and optionally a second composition, comprising a rAAV encoding LAMP2.

In one embodiment, the method includes administering to a human with Fabry or Anderson-Fabry disease a first composition and optionally a second composition, comprising a rAAV encoding alpha-galactosidase A.

In one embodiment, the method includes administering to a human with Type I or Type 3, 3C, Gaucher Disease a first composition and optionally a second composition, comprising a rAAV encoding glucocerebrosidase.

In one embodiment, the method includes administering to a human with sialidosis (Mucolipidosis I) a first composition and optionally a second composition, comprising a rAAV encoding sialidase.

In one embodiment, the method includes administering to a human with mucolipidosis II (I-cell disease) a first composition and optionally a second composition, comprising a rAAV encoding N-acetylglucosamine-1-phosphotransferase.

In one embodiment, the method includes administering to a human with mucolipidosis III (pseudo-Hurler polydystrophy) a first composition and optionally a second composition, comprising a rAAV encoding N-acetylglucosamine-1-phosphotransferase.

In one embodiment, the method includes administering to a human with aspartylglucosaminuria a first composition and optionally a second composition, comprising a rAAV encoding aspartylglucosamidase.

In one embodiment, the method includes administering to a human with fucosidosis a first composition and optionally a second composition, comprising a rAAV encoding fucosidase.

In one embodiment, the method includes administering to a human with mannosidosis (Alpha & Beta) a first composition and optionally a second composition, comprising a rAAV encoding mannosidase.

In one embodiment, the method includes administering to a human with pycnodysostosis a first composition and optionally a second composition, comprising a rAAV encoding cathepsin K.

In one embodiment, the method includes administering to a human with galactosialidosis a first composition and optionally a second composition, comprising a rAAV encoding galactosidase.

In one embodiment, the method includes administering to a human with multiple sulfatase deficiency (MSD) a first composition and optionally a second composition, comprising a rAAV encoding SUMF-1.

In one embodiment, the method includes administering to a human with Farber disease a first composition and optionally a second composition, comprising a rAAV encoding N-acylsphingosine amidohydrolase (ASAH1).

In one embodiment, the amount reduces, treats, inhibits, or prevents cardiac valve dysfunction, coronary artery abnormalities, myocardial abnormalities, conduction system abnormalities, and/or aortic root dilation. In one embodiment, the amount reduces, treats, inhibits, or prevents cardiac dysfunction such as left ventricular hypertrophy, cardiac failure, hypertrophic cardiomyopathy, progressive conduction system disease, vulvular regurgitation, stenosis, cardiac hypertrophy, systolic dysfunction, pulmonary hypertension, pericardial effusion, and/or arrhythmias. In one embodiment, the mammal has one or more of cardiac valve disease, coronary artery abnormalities, myocardial abnormalities, conduction system abnormalities, or aortic root dilation. In one embodiment, administration inhibits progression of mitral valve dysfunction, aortic valve dysfunction, valve regurgitation, and/or stenosis, e.g., which results in thickened valve. In one embodiment, administration inhibits progression of systolic dysfunction, diastolic dysfunction, or both. In one embodiment, the amount reduces, treats, inhibits or prevents skeletal abnormalities such as short stature, dysostosis, contracture, hip dysplasia, scoliosis, kyphosis, low bone density, fracture, avascular necrosis, bone crisis and/or osteosclerosis

In one embodiment, the mammal is treated with one or more immunosuppressants (e.g., corticosteroids, tacrolimus and/or sirolumus). In one embodiment, at least one the rAAVs and the immune suppressant are co-administered, or the immune suppressant is administered after at least one of the rAAVs, or the immune suppressant is administered before at least one of the rAAVs. In one embodiment, the mammal is not treated with an immunosuppressant. In one embodiment, the mammal is not immunotolerized prior to administration of at least one of the rAAVs. In one embodiment, the mammal is immunotolerized prior to administration of rAAV.

In one embodiment, the mammal is a human. In one embodiment, the human has been diagnosed with MPS I. In one embodiment, the human has been diagnosed with MPS II. In one embodiment, the rAAV (e.g. rAAV9) comprising an open reading frame encoding a gene product (e.g., IDUA or IDS) is administered to the human via the IT/IC route of administration in a dose range of about 1×10⁸ to 1×10¹³ GC/g brain, 1×10⁹ to 1×10¹¹ GC/g brain, or 1×10¹⁰ to 1×10¹² GC/g brain. In one embodiment, the rAAV (e.g. rAAV9) comprising an open reading frame encoding a gene product (e.g., IDS) is administered to the human via IT/IC route of administration at a dose of about 1.3×10¹⁰, 6.5×10¹⁰ or 2×10¹¹ GC/g brain. In one embodiment, the rAAV (e.g. rAAV9) comprising an open reading frame encoding a gene product (e.g., IDUA) is administered to the human via IT/IC route of administration at a dose of about 1.3×10¹⁰, 5.0×10¹⁰ or 2×10¹¹ GC/g brain. In one embodiment, the rAAV (e.g. rAAV9) comprising an open reading frame encoding a gene product (e.g., IDUA or IDS) is administered to the human via IV route of administration in a dose range of about 1×10⁸ to 1×10¹³ GC/kg, 1×10⁹ to 1×10¹¹ GC/kg, or 1×10¹⁰ to 1×10¹² GC/kg, e.g., about 2×10¹¹ GC/kg to about 8×10¹¹ GC/kg or about 5×10¹¹ GC/kg to about 1×10^{12s} GC/kg. In one embodiment, the rAAV (e.g. rAAV9) comprising an open reading frame encoding a gene product (e.g., IDUA or IDS) is administered to the human via the IV route of administration at a dose of about 1×10¹¹ GC/kg, 2×10¹¹ GC/kg, 3×10¹¹ GC/kg, 4×10¹¹ GC/kg, 5×10¹¹ GC/kg, 6×10¹¹ GC/kg, 7×10¹¹ GC/kg, 8×10¹¹ GC/kg, 9×10¹¹ GC/kg, or 1×10¹² GC/kg. In one embodiment human is administered the rAAV (e.g. rAAV9) comprising an open reading frame encoding a gene product (e.g., IDUA or IDS) via two routes of administration (e.g., IV and IT/IC) at the aforementioned dose ranges and doses described for IT/IC administration and IV administration, respectively.

In one embodiment the human treated in accordance with the methods of the disclosure is one month old, two months old, three months old, four months old, five months old, six months old, seven months old, eight months old, none months old, ten months old, eleven months old, 1 year old, two years old, three years old, four years old, five years old, six years old, seven years old, eight years old, nine years old, ten years old, eleven years old, twelve years old, thirteen years old, fourteen years old, fifteen years old, sixteen years old, seventeen years old, eighteen years old or an adult older than eighteen years old.

In one embodiment, at least one of the rAAV vector is a recombinant AAV1, AAV2, AAV2tYF, AAV3, AAV4, AAV5, AAV6, AAV7, AAV8, AAV9, AAV10, AAV11, and AAVrh10, AAV.rh20, AAV.rh39, AAV.Rh74, AAV.RHM4-1, AAV.hu37, AAV.Anc80, AAV.Anc80L65, rAAV.7m8, AAV.PHP.B, AAV.PHP.eB, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, or AAV.HSC16 vector. In one embodiment, at least one of the rAAVs is rAAV9 or rAAVrh10. In one embodiment, at least one of the rAAVs is rAAV9. In one embodiment, at least one of the rAAVs is rAAVrh10. In one embodiment, the rAAV encodes IDUA, IDS, SGSH, NAGLU, HGSNAT, GNS, GALNS, GLB1, ARSB, GUSB, HYAL1, alpha-glycosidase, LAMP2, alpha-galactosidase A, glucocerebrosidase, sialidase, N-acetylglucosamine-1-phosphotransferase, N-acetylglucosamine-1-phosphotransferazse, aspartylglucosamidase, fucosidase, mannosidase, cathepsin K, galactosidase, SUMF-1, or N-acylsphingosine amidohydrolase (ASAH1).

Diseases or disorders amenable to the AAV therapy disclosed herein include diseases or disorders associated with cardiac defects, including but not limited to: MPS I, MPS II, MPS III (3a, 3b, 3c, 3d), MPS IV (4a, 4b), MPS VI, MPS VII, Pompe disease, Danon disease, Anderson-Fabry diseases, or Type 3C Gaucher Disease,

Diseases or disorders amenable to the AAV therapy disclosed herein include diseases or disorders associated with skeletal defects, including but not limited to: MPS I, MPS II, MPS III (3a, 3b, 3c, 3d), MPS IV (4a, 4b), MPS VI, MPS VII, MPS IX, Gaucher Disease (Type 1 and Type 3), Sialidosis (Mucolipidosis I), Mucolipidosis II (1-cell disease), Mucolipidosis III (pseudo-Hurler polydystrophy), Aspartylglucosaminuria, Fucosidosis, Mannosidosis (Alpha & Beta), Pycnodysostosis, Galactosialidosis, MSD, Pompe, Farber, or Fabry disease.

Diseases or disorders amenable to the therapy, e.g., AAV therapy, disclosed herein include diseases or disorders associated with a deficiency in a protein, diseases/disorders and proteins including but not limited to MPS I and alpha iduronidase, MPS II and iduronate-2-sulfatase, MPS III (3a, 3b, 3c, or 3d) and heparan-N-sulfatase, alpha-N-acetyl-glucosamindase, acetyl CoA:alpha-glucosaminide-acetyltransferase, or N-acetylgluycosamine-6-sulfatase, MPS IV (4a or 4b) and N-acetylgluycosamine-6-sulfatase or beta-galactosidase, MPS VI and N-acetylgalactosamine-4-sulphatase, MPS VII and beta-glucuronidase, Pompe disease and alpha-glycosidase, Danon disease and LAMP2, Anderson-Fabry disease and alpha-galactosidase A, or Type 3C Gaucher Disease and glucocerebrosidase.

Diseases or disorders amenable to the therapy, e.g., AAV therapy disclosed herein include diseases or disorders associated with a deficiency in a protein, diseases/disorders and proteins including but not limited to MPS IX and hyaluronidase, Gaucher Disease (Type 1 or Type 3) and glucocerebrosidase, sialidosis (Mucolipidosis I) and sialidase, mucolipidosis 11 (1-cell disease) and N-acetylglucosamine-1-phosphotransferase, mucolipidosis III (pseudo-Hurler polydystrophy) and N-acetylglucosamine-1-phosphotransferazse, aspartylglucosaminuria and aspartylglucosamidase, fucosidosis and fucosidase, mannosidosis (Alpha & Beta) and mannosidase, pycnodysostosis and cathepsin K, galactosialidosis and galactosidase, multiple sulfatase deficiency (MSD) and SUMF-1, Farber disease and N-acylsphingosine amidohydrolase (ASAH1), or Fabry disease.

Diseases or disorders amenable to the AAV therapy disclosed herein include diseases or disorders associated with a deficiency in a protein which can be supplied by the transgene used in the therapy. Such diseases/disorders associated with a deficiency in certain proteins include but are not limited to MPS I and alpha iduronidase, MPS II and iduronate-2-sulfatase, MPS III (IIIA, IIIb, IIIC, or IIID) and heparan-N-sulfatase, alpha-N-acetyl-glucosamindase, acetyl CoA:alpha-glucosaminide-acetyltransferase, or N-acetylgluycosamine-6-sulfatase, MPS IV (IVA or IVB) and N-acetylgluycosamine-6-sulfatase or beta-galactosidase, MPS VI and N-acetylgalactosamine-4-sulphatase, MPS VII and beta-glucuronidase, Pompe disease and alpha-glycosidase, Danon disease and LAMP2, Anderson-Fabry disease and alpha-galactosidase A, or Type 3C Gaucher Disease and glucocerebrosidase.

Diseases or disorders amenable to the AAV therapy disclosed herein include diseases or disorders associated with a deficiency in a protein which can be supplied by the transgene used in the therapy. Such diseases/disorders associated with a deficiency in certain proteins include but are not limited to MPS IX and hyaluronidase, Gaucher Disease (Type 1 or Type 3) and glucocerebrosidase, sialidosis (Mucolipidosis I) and sialidase, mucolipidosis II (I-cell disease) and N-acetylglucosamine-1-phosphotransferase, mucolipidosis III (pseudo-Hurler polydystrophy) and N-acetylglucosamine-1-phosphotransferazse, aspartylglucosaminuria and aspartylglucosamidase, fucosidosis and fucosidase, mannosidosis (Alpha & Beta) and mannosidase, pycnodysostosis and cathepsin K, galactosialidosis and galactosidase, multiple sulfatase deficiency (MSD) and SUMF-1, Farber disease and N-acylsphingosine amidohydrolase (ASAH1), or Fabry disease.

In one embodiment, the disclosure provides for delivery of therapeutic proteins via rAAV to prevent, inhibit the progression of, reduce the severity of, or treat cardiac, vascular or skeletal dysfunction or defects in a mammal, e.g., a mammal having MPSI or MPSII. In one embodiment, rAAV is delivered to a mammal intrathecally (IT), endovascularly (IV), or cerebroventricularly (ICV), or a combination thereof, to prevent, inhibit the progression of, reduce the severity of, or treat cardiac, vascular or skeletal dysfunction or defects. In one embodiment, the mammal is subjected to immunosuppression. In one embodiment, the mammal is subjected to tolerization. In one embodiment, methods of preventing, inhibiting, and/or treating cardiac, vascular or skeletal dysfunction or defects in, for example, an adult mammal or a neonate, are provided. The methods involve delivering to a mammal in need of treatment a composition comprising an effective amount of a recombinant adeno-associated virus (rAAV) vector comprising an open reading frame encoding IDUA or IDS. The AAV vector can be administered in a variety of ways to ensure that it is delivered and that the transgene is successfully transduced. Routes of delivery include, but are not limited to intrathecal administration, intracisternal administration, intracranial administration, e.g., intracerebroventricular administration, or lateral cerebroventricular administration, administration, intravascular administration, intravenous administration, endovascular administration, and intraparenchymal administration. In one embodiment, the amount of AAV-IDUA or AAV-IDS administered results in an increase, e.g., 2-, 5-, 10-, 25-, 50-, 100-, 200- or 500-fold or more, up to 1000-fold more IDS or IDUA, e.g., in plasma, the heart, bone, or the brain, in the adult mammal relative to a corresponding mammal with MPSI or MPSII that is not administered the AAV-IDUA or AAV-IDS.

In one embodiment, the methods involve delivering to an adult mammal in need of treatment a composition comprising an effective amount of a rAAV serotype 9 (rAAV9) vector comprising an open reading frame encoding IDS or IDUA. In one embodiment, the methods involve delivering to an adult mammal in need of treatment a composition comprising an effective amount of a rAAV9 vector comprising an open reading frame encoding IDS and optionally one encoding SUMF-1. For example, AAV9-IDS may be administered by direct injection into a mammal that is either immunocompetent, immunodeficient, immunosuppressed, e.g., with cyclophosphamide (CP), or immunotolerized by injection of IDUA or IDS protein. In one embodiment, the amount of AAV-IDUA or rAAV-IDS administered results in an increase, e.g., 2-, 5-, 10-, 25-, 50-, 100-, 200- or 500-fold or more, up to 1000-fold more IDS or IDS, e.g., in plasma, the heart, bone or the brain, in the adult mammal relative to a corresponding mammal with MPSI or MPSII that is not administered the AAV-IDS or AAV-IDUA.

Thus, the disclosure includes the use of recombinant AAV (rAAV) vectors that encode a gene product with therapeutic effects when expressed in a mammal. In one embodiment, the mammal is an immunocompetent mammal with a disease or disorder of cardiac, vascular or skeletal tissue. An “immunocompetent” mammal as used herein is a mammal of an age where both cellular and humoral immune responses are elicited after exposure to an antigenic stimulus, by upregulation of Th1 functions or IFN-γ production in response to polyclonal stimuli, in contrast to a neonate which has innate immunity and immunity derived from the mother, e.g., during gestation or via lactation. An adult mammal that does not have an immunodeficiency disease is an example of an immunocompetent mammal. For example, an immunocompetent human is typically at least 1, 2, 3, 4, 5 or 6 months of age, and includes adult humans without an immunodeficiency disease. In one embodiment, the AAV is administered intrathecally. In one embodiment, the AAV is administered intracranially (e.g., intracerebroventricularly). In one embodiment, the AAV is administered, with or without a permeation enhancer. In one embodiment, the AAV is administered endovascularly, e.g., carotid artery administration, with or without a permeation enhancer. In one embodiment, the mammal that is administered the AAV is immunodeficient or is subjected to immunotolerization or immune suppression, e.g., to induce higher levels of therapeutic protein expression relative to a corresponding mammal that is administered the AAV but not subjected to immunotolerization or immune suppression. In one embodiment, an immune suppressive agent is administered to induce immune suppression. In one embodiment, the mammal that is administered the AAV is not subjected to immunotolerization or immune suppression (e.g., administration of the AAV alone provides for the therapeutic effect). In one embodiment, the amount of AAV-IDUA administered results in an increase, e.g., 2-, 5-, 10-, 25-, 50-, 100-, 200- or 500-fold or more, up to 1000-fold more IDS, e.g., in plasma or the brain, in the adult mammal relative to a corresponding mammal with MPSII that is not administered the AAV-IDS.

In one embodiment, the disclosure provides a method to augment secreted protein in a mammal having cardiac, vascular or skeletal dysfunction or defect(s). The method includes administering to the mammal a composition comprising an effective amount of a recombinant adeno-associated virus (rAAV) vector comprising an open reading frame encoding the secreted protein, the expression of which in the mammal reduces cardiac, vascular or skeletal dysfunction or defects, and optionally enhances neurocognitive function, relative to a mammal with the disease or dysfunction but not administered the rAAV. In one embodiment, the rAAV or a different rAAV encodes an antibody. In one embodiment, the mammal is not treated with an immunosuppressant. In another embodiment, for example, in subjects that may generate an immune response that neutralizes activity of the therapeutic protein, the mammal is treated with an immunosuppressant, e.g., a glucocorticoid, cytostatic agents including an alkylating agent, an anti-metabolite, a cytotoxic antibiotic, an antibody, or an agent active on immunophilin, such as a nitrogen mustard, nitrosourea, platinum compound, methotrexate, azathioprine, mercaptopurine, fluorouracil, dactinomycin, an anthracycline, mitomycin C, bleomycin, mithramycin, IL-2 receptor-(CD25-) or CD3-directed antibodies, anti-IL-2 antibodies, ciclosporin, tacrolimus, sirolimus, IFN-β, IFN-γ, an opioid, or TNF-α (tumor necrosis factor-alpha) binding agent. In one embodiment, the rAAV and the immune suppressant are co-administered or the immune suppressant is administered after the rAAV. In one embodiment, the immune suppressant is intrathecally administered. In one embodiment, the immune suppressant is intracerebroventricularly administered. In one embodiment, the rAAV vector is a rAAV1, rAAV3, rAAV4, rAAV5, rAAVrh10, or rAAV9 vector. In one embodiment, prior to administration of the composition the mammal is immunotolerized. In one embodiment, the amount of AAV-IDUA administered results in an increase, e.g., 2-, 5-, 10-, 25-, 50-, 100-, 200- or 500-fold or more, up to 1000-fold more IDUA, e.g., in plasma, heart, bone, or brain, in the adult, neonate or juvenile mammal relative to a corresponding mammal with MPSI that is not administered the AAV-IDUA.

In one embodiment, the disclosure provides a method to prevent, inhibit the progression of, reduce the severity of, or treat cardiac, vascular or skeletal dysfunction or defects in a mammal. The method includes administering to the mammal a composition comprising an effective amount of a recombinant adeno-associated virus (rAAV) vector comprising an open reading frame encoding an IDS. In one embodiment, the amount of AAV-IDS administered results in an increase, e.g., 2-, 5-, 10-, 25-, 50-, 100-, 200- or 500-fold or more, up to 1000-fold more IDS, e.g., in plasma, heart, bone, or brain, in the adult, neonate or juvenile mammal relative to a corresponding mammal with MPSII that is not administered the AAV-IDS.

In one embodiment, a method to prevent, inhibit the progression of, reduce the severity of, or treat cardiac, vascular or skeletal dysfunction or defects, in a mammal with a lysosomal storage disorder such as MPSI or MPSII is provided The method includes intrathecally, e.g., to the lumbar region, or intracerebroventricularly, e.g., to the lateral ventricle, administering to the mammal a composition comprising an effective amount of a rAAV vector comprising an open reading frame encoding an IDS or IDUA, the expression of which in the central nervous system of the mammal enhances or restores neurocognitive function, and systemically administering to the mammal a composition comprising an effective amount of a rAAV vector comprising an open reading frame encoding an IDS or IDUA, the expression of which in the mammal prevents, inhibits or treats cardiac, vascular or skeletal dysfunction or defects. In one embodiment, the mammal is an immunocompetent adult. In one embodiment, the rAAV vector is an AAV1, AAV2, AAV3, AAV4, AAV5, AAV6, AAV7, AAV8, AAVrh10, or AAV9 vector. In one embodiment, the mammal is a human. In one embodiment, multiple doses are administered. In one embodiment, the composition is administered weekly, monthly or two or more months apart. In one embodiment, the amount of AAV-IDUA or AAV-IDS administered results in an increase, e.g., 2-, 5-, 10-, 25-, 50-, 100-, 200- or 500-fold or more, up to 1000-fold more IDS, e.g., in plasma, heart, bone, or brain, in the adult mammal relative to a corresponding mammal with MPSI that is not administered AAV-IDUA or MPSII that is not administered the AAV-IDS. In one embodiment, the amount of AAV-IDUA or AAV-IDS administered results in an increase, e.g., 2-, 5-, 10-, 25-, 50-, 100-, 200- or 500-fold or more, up to 1000-fold more IDS, e.g., in plasma, heart, bone, or brain, in the neonate mammal relative to a corresponding neonate mammal with MPSI that is not administered AAV-IDUA or MPSII that is not administered the AAV-IDS. In one embodiment, the amount of AAV-IDUA or AAV-IDS administered results in an increase, e.g., 2-, 5-, 10-, 25-, 50-, 100-, 200- or 500-fold or more, up to 1000-fold more IDS, e.g., in plasma, heart, bone, or brain, in the juvenile mammal relative to a corresponding juvenile mammal with MPSI that is not administered AAV-IDUA or MPSII that is not administered the AAV-IDS.

In one embodiment, the method includes intrathecally, e.g., to the lumbar region, administering to a mammal a composition comprising an effective amount of a rAAV vector comprising an open reading frame encoding an IDS, and optionally administering a permeation enhancer. In one embodiment, the permeation enhancer is administered before the composition. In one embodiment, the composition comprises a permeation enhancer. In one embodiment, the permeation enhancer is administered after the composition. In one embodiment, the mammal is an immunocompetent adult. In one embodiment, the rAAV vector is an AAV-1, AAV-3, AAV-4, AAV-5, AAV-6, AAV-7, AAV-8, AAV rh10, or AAV-9 vector. In one embodiment, the mammal is a human. In one embodiment, multiple doses are administered. In one embodiment, the composition is administered weekly, monthly or two or more months apart. In one embodiment, the mammal that is intrathecally administered the AAV is not subjected to immunotolerization or immune suppression (e.g., administration of the AAV alone provides for the therapeutic effect). In one embodiment, the mammal that is intrathecally administered the AAV is immunodeficient or is subjected to immunotolerization or immune suppression, e.g., to induce higher levels of therapeutic protein expression relative to a corresponding mammal that is intrathecally administered the AAV but not subjected to immunotolerization or immune suppression. In one embodiment, the amount of AAV-IDUA administered results in an increase, e.g., 2-, 5-, 10-, 25-, 50-, 100-, 200- or 500-fold or more, up to 1000-fold more IDS, e.g., in plasma or the brain, in the adult mammal relative to a corresponding mammal with MPSII that is not administered the AAV-IDS.

In one embodiment, the method includes intracerebroventricularly, e.g., to the lateral ventricle, administering to an immunocompetent mammal a composition comprising an effective amount of a rAAV vector comprising an open reading frame encoding an IDS. In one embodiment, the rAAV vector is an AAV1, AAV2, AAV3, AAV4, AAV5, AAV6, AAV7, AAV8, AAVrh10, or AAV9 vector. In one embodiment, the rAAV vector is not a rAAV5 vector. In one embodiment, the mammal is a human. In one embodiment, multiple doses are administered. In one embodiment, the composition is administered weekly, monthly or two or more months apart. In one embodiment, the mammal that is intracerebroventricularly administered the AAV is not subjected to immunotolerization or immune suppression (e.g., administration of the AAV alone provides for the therapeutic effect). In one embodiment, the mammal that is intracerebroventricularly administered the AAV is immunodeficient or is subjected to immunotolerization or immune suppression, e.g., to induce higher levels of therapeutic protein expression relative to a corresponding mammal that is intracerebroventricularly administered the AAV but not subjected to immunotolerization or immune suppression In one embodiment, the mammal is immunotolerized to the gene product before the composition comprising the AAV is administered. In one embodiment, the amount of AAV-IDUA administered results in an increase, e.g., 2-, 5-, 10-, 25-, 50-, 100-, 200- or 500-fold or more, up to 1000-fold more IDS, e.g., in plasma or the brain, in the adult mammal relative to a corresponding mammal with MPSII that is not administered the AAV-IDS.

Further provided is a method to prevent, inhibit the progression of, reduce the severity of, or treat cardiac, vascular or skeletal dysfunction or defects, in a mammal having MPSII. The method includes endovascularly administering to the mammal a composition comprising an effective amount of a rAAV vector comprising an open reading frame encoding an IDS. In one embodiment, the mammal is an immunocompetent adult. In one embodiment, the rAAV vector is an AAV1, AAV2, AAV3, AAV4, AAV5, AAV6, AAV7, AAV8, AAVrh10, or AAV9 vector. In one embodiment, the rAAV vector is not a rAAV5 vector. In one embodiment, the mammal is a human. In one embodiment, multiple doses are administered. In one embodiment, the composition is administered weekly. In one embodiment, the composition is administered weekly, monthly or two or more months apart. In one embodiment, the mammal that is endovascularly administered the AAV is not subjected to immunotolerization or immune suppression (e.g., administration of the AAV provides for the therapeutic effect). In one embodiment, the mammal that is endovascularly administered the AAV is immunodeficient or is subjected to immunotolerization or immune suppression, e.g., to induce higher levels of therapeutic protein expression relative to a corresponding mammal that is endovascularly administered the AAV but not subjected to immunotolerization or immune suppression. In one embodiment, the amount of AAV-IDUA administered results in an increase, e.g., 2-, 5-, 10-, 25-, 50-, 100-, 200- or 500-fold or more, up to 1000-fold more IDS, e.g., in plasma or the brain, in the adult mammal relative to a corresponding mammal with MPSII that is not administered the AAV-IDS.

In one embodiment, the method includes administering to an adult mammal a composition comprising an effective amount of a rAAV9 vector comprising an open reading frame encoding an IDS. In one embodiment, the mammal is an immunocompetent adult. In one embodiment, the mammal is a human. In one embodiment, multiple doses are administered. In one embodiment, the composition is administered weekly, monthly or two or more months apart. In one embodiment, the mammal that is administered the AAV is not subjected to immunotolerization or immune suppression. In one embodiment, the mammal that is administered the AAV is subjected to immunotolerization or immune suppression, e.g., to induce higher levels of IDUA protein expression relative to a corresponding mammal that is administered the AAV but not subjected to immunotolerization or immune suppression. In one embodiment, the amount of AAV-IDUA administered results in an increase, e.g., 2-, 5-, 10-, 25-, 50-, 100-, 200- or 500-fold or more, up to 1000-fold more IDS, e.g., in plasma or the brain, in the adult mammal relative to a corresponding mammal with MPSII that is not administered the AAV-IDS.

Further provided is a system or kit comprising an amount of a rAAV vector comprising an open reading frame encoding an IDS for IT administration and/or an amount of a rAAV vector comprising an open reading frame encoding an IDS for systemic, e.g., IV, administration. In one embodiment, the system or kit comprises a composition comprising an amount of a rAAV vector comprising an open reading frame encoding an IDS in a pharmaceutical carrier suitable for IT administration. In one embodiment, the system or kit comprises a composition comprising an amount of a rAAV vector comprising an open reading frame encoding an IDS in a pharmaceutical carrier suitable for IV administration. In one embodiment, the system or kit comprises a composition comprising an amount of a rAAV vector comprising an open reading frame encoding an IDS in a pharmaceutical carrier suitable for IT administration and a composition comprising an amount of a rAAV vector comprising an open reading frame encoding an IDS in a pharmaceutical carrier suitable for IV administration.

Routes of administration include, but are not limited to intrathecal administration, intracisternal administration, intracranial administration, e.g., intracerebroventricular administration or lateral cerebroventricular administration, administration, endovascular administration, intravenous administration, or intraparenchymal administration. In one embodiment, the amount of AAV-IDS administered results in an increase, e.g., 2-, 5-, 10-, 25-, 50-, 100-, 200- or 500-fold or more, up to 1000-fold more IDS, e.g., in plasma or the brain, in the adult mammal relative to a corresponding mammal with MPSII that is not administered the AAV-IDS.

In one embodiment, the human to be treated is 1, 2, 3, 4, 5, 6, 7, 8, 9, 10, or 11 months old, 1, 2, 3, 4, 5, 6, 7, 8, 9, 10, 11, 12, 13, 14, 15, 16, 17 or 18 years old, or an adult, e.g., a human over the age of 18.

Other viral vectors may be employed in the methods, e.g., viral vectors such as retrovirus, lentivirus, adenovirus, semliki forest virus or herpes simplex virus vectors.

### BRIEF DESCRIPTION OF THE FIGURES

FIGS. 1A-1B. Plasma IDUA activity in males (A) and females (B).

FIGS. 2A-2C. Echocardiography results for ascending aorta diameter (A), shortening fraction (B) and aortic insufficiency (C).

FIG. 3. Gene therapy for MPSI.

FIG. 4. Exemplary experimental protocol and timeline.

FIG. 5. Supraphysiological levels of IDUA observed in plasma and PBMC FIG. 6. Reduction of urine GAGs.

FIG. 7. High IDUA enzyme activity in male tissues. Animals were euthanized at 8 months of age (6 months post-treatment), and tissue lysates were analyzed for IDUA enzyme activity and GAG storage.

FIG. 8. High IDUA enzyme activity in female tissues. Animals were euthanized at 8 months of age (6 months post-treatment), and tissue lysates were analyzed for IDUA enzyme activity and GAG storage.

FIG. 9. Normalization of GAGs. Animals were euthanized at 8 months of age (6 months post-treatment), and tissue lysates were analyzed for IDUA enzyme activity and GAG storage.

FIG. 10. Restoration of neurocognitive learning in treated animals. Animals were evaluated with behavioral testing at 5 months of age (3 months post-treatment). The BM measures spatial memory and navigation which is a function of the hippocampus. The animal is trained to locate an escape hole on the maze, and time taken to locate the escape hole is recorded as latency to escape. The test is carried out over 4 days with 4 trials per day, and as you can see, it takes them less time to locate the escape hole on day 4 than on day 1. FC is a function of associative learning that is primarily associated with the hippocampus, but also other parts of the brain. In this test, we measure the fear response after administration of a mild foot shock.

FIG. 11. Improved cardiac function. Data suggest that valvulopathy in MPS I mice was attenuated following AAV9.IDUA treatment.

FIG. 12. MicroCT analyses of skull diameter (females). Analyses of untreated MPSI mice, heterozygotes, MPSI mice treated with AAV IV, MPSI mice treated with AAV IT, or MPSI mice subjected to combination treatment (IV and IT).

FIG. 13. MicroCT analyses of zygomatic arch diameter. Analyses of untreated MPSI mice, heterozygotes, MPSI mice treated with AAV IV, MPSI mice treated with AAV IT, or MPSI mice subjected to combination treatment (IV and IT).

FIG. 14. MicroCT analyses of skull diameter. Analyses of untreated MPSI mice, heterozygotes, MPSI mice treated with AAV IV, MPSI mice treated with AAV IT, or MPSI mice subjected to combination treatment (IV and IT).

FIG. 15. MicroCT analyses of femur diameter. Analyses of untreated MPSI mice, heterozygotes, MPSI mice treated with AAV IV, MPSI mice treated with AAV IT, or MPSI mice subjected to combination treatment (IV and IT).

FIG. 16. MicroCT analyses of spine angle. Analyses of untreated MPSI mice, heterozygotes, MPSI mice treated with AAV IV, MPSI mice treated with AAV IT, or MPSI mice subjected to combination treatment (IV and IT).

FIG. 17. In vivo AAV9 gene therapy for MPS II in a mouse model. CB7 is CMV immediate early enhancer, chicken beta promoter and intron. Rabbit globin splice acceptor/exon 3. cohIDS, rabbit globin pA.

FIGS. 18A-18B. Plasma IDS enzyme level dose response in RGX-121 treated mice (IT (A) and IV (B)).

FIGS. 19A-19B. High level IDS in tissues of RGX-121 treated mice (IT (A) and IV (B)).

FIGS. 20A-20B. GAG normalization in tissues of RGX-121 treated mice (IT (A) and IV (B)).

FIGS. 21A-21B. Biodistribution in RGX-121 treated mice (IT (A) and IV (B).

FIGS. 22A-22B. RGX-121 normalizes zygomatic arch diameter (IT (A) and IV (B)).

FIG. 23. Prevention of neurocognitive deficit in RGX-121 treated mice.

FIGS. 24A-24B. Plasma IDUA activity (10¹⁰) (males (A), females (B)).

FIG. 25. Urine GAG (10¹⁰).

FIG. 26. Barnes maze.

FIG. 27. Fear conditioning: Day 1.

FIG. 28. Fear conditioning: Day 2. Results from Fear Conditioning indicate that the % difference in freezing is significantly different for the IV+IT group compared to untreated animals. Similar to results from the Barnes maze, the IV+IT route appears to be most effective in reducing neurocognitive deficit.

FIGS. 29A-29C. Echo results. Results from ECHO indicate that IV and IV+IT routes have significantly beneficial effects on parameters 1 and 3 respectively, compared to untreated controls. IT: 4 out of 6 had no AI; 1 had trace AI; 1 had AI. IV: 4 out of 6 had no AI; 1 had trace AI; 1 had AI; IV+IT: 5 out of 5 had no AI; MPS I: 4 out of 6 had AI.

FIG. 30. Skeletal effects.

FIG. 31. Skeletal effects.

FIG. 32. Tissue IDUA activity.

FIG. 33. Tissue IDUA activity.

FIG. 34. Tissue GAG.

FIG. 35. IDUA activity (Intrathecal dose response).

FIG. 36. Urine GAG.

FIG. 37. Tissue GAG (Intrathecal dose response)

FIG. 38. IDUA activity (Intravenous dose response).

FIG. 39. Tissue GAG (Intravenous Dose Response).

FIG. 40. Neurocognitive testing: RC11 (IT).

FIG. 41. Neurocognitive testing: RC12 (IV).

FIGS. 42A-42C. Echo results.

FIGS. 43A-43B. Q-PCR.

FIGS. 44A-44B. Plasma IDS activity: dose comparison (IT (A) and IV (B)).

FIGS. 45A-45D. Urine GAG excretion: higher dose comparison.

FIGS. 46A-46B. Tissue IDS activity (IT (A) and IV (B)).

FIGS. 47A-47B. Brain IDS activity (IT (A) and IV (B)).

FIGS. 48A-48B. Tissue GAG accumulation (IT (A) and IV (B)).

FIGS. 49A-49B. Brain GAG accumulation (IT (A) and IV (B)).

FIGS. 50A-50B. High and lose dose biodistribution (qPCR) (IT (A) and IV (B)).

FIGS. 51A-51B. Brain biodistribution (qPCR) (IT (A) and IV (B)).

FIGS. 52A-52B. Skeletal analysis: Zygomatic arch diameter (IT (A) and IV (B)).

FIGS. 53A-53B. Skeletal analysis: Skull width (IT (A) and IV (B)).

FIGS. 54A-54B. Skeletal analysis: Snout length (IT (A) and IV (B)).

FIGS. 55A-55B. Skeletal analysis: Angle of spine curvature (IT (A) and IV (B)).

FIGS. 56A-56B. Skeletal analysis: Femur length (IT (A) and IV (B)).

FIGS. 57A-57B. Skeletal analysis: Femur diameter (IT (A) and IV (B)).

FIGS. 58A-58C. Neurobehavioral studies: Higher dose IT, IV Barnes maze. IT (A), IV (B), IT+IV (C).

FIG. 59. Comparison of ROA and doses two weeks post-treatment in males and females.

FIG. 60. IDUA activity in tissues.

FIG. 61. IDUA activity. No reduction in urine GAGs was observed with 10⁷ and 10⁸ vg administered either IT or IV.

FIG. 62. GAG activity.

FIG. 63. GAG activity in tissues.

FIG. 64. Graph of latency to escape for IT and IV for all ROAs.

FIG. 65. Graph differences in freezing for IT and IV for all ROAs.

FIG. 66. Ascending aortic diameter and aortic insufficiency based on dose and/or route. I=Aortic Insufficiency present. 0=No aortic insufficency. Data suggest that valvulopathy in MPS I mice was attenuated following AAV9-IDUA treatment.

FIG. 67. Femur length and skull diameter based on dose and/or route.

FIG. 68. Latency to escape and differences in freezing for IT and IV.

FIG. 69. Measurements in females and males.

FIG. 70. Measurements in males.

FIG. 71. Measurements in males.

FIG. 72. Measurements in males.

FIG. 73. Plasma IDS ACTVITY: 10⁹ IT+IV.

### DETAILED DESCRIPTION

### Definitions

As used herein, “individual” (as in the subject of the treatment) means a mammal. Mammals include, for example, humans; non-human primates, e.g., apes and monkeys; and non-primates, e.g., dogs, cats, rats, mice, cattle, horses, sheep, and goats. Non-mammals include, for example, fish and birds.

The term “disease” or “disorder” are used interchangeably, and are used to refer to diseases or conditions wherein lack of or reduced amounts of a specific gene product, e.g., a lysosomal storage enzyme, plays a role in the disease such that a therapeutically beneficial effect can be achieved by supplementing, e.g., to at least 1% of normal levels.

“Substantially” as the term is used herein means completely or almost completely; for example, a composition that is “substantially free” of a component either has none of the component or contains such a trace amount that any relevant functional property of the composition is unaffected by the presence of the trace amount, or a compound is “substantially pure” is there are only negligible traces of impurities present.

“Treating” or “treatment” within the meaning herein refers to an alleviation of or improvement in symptoms associated with a disorder or disease, “inhibiting” means inhibition of further progression or worsening of the symptoms associated with the disorder or disease, and “preventing” refers to prevention of the symptoms associated with the disorder or disease.

As used herein, an “effective amount” or a “therapeutically effective amount” of an agent of the disclosure, e.g., a recombinant AAV encoding a gene product, refers to an amount of the agent that alleviates or improves, in whole or in part, symptoms associated with the disorder or condition, or halts or slows further progression or worsening of those symptoms, or prevents or provides prophylaxis for the disorder or condition, e.g., an amount that is effective to prevent, inhibit the progression of, reduce the severity of, or treat in the individual one or more cardiac, vascular or skeletal symptoms.

In particular, a “therapeutically effective amount” refers to an amount effective, at dosages and for periods of time necessary, to achieve the desired therapeutic result. A therapeutically effective amount is also one in which any toxic or detrimental effects of compounds of the disclosure are outweighed by the therapeutically beneficial effects.

A “vector” as used herein refers to a macromolecule or association of macromolecules that comprises or associates with a polynucleotide and which can be used to mediate delivery of the polynucleotide to a cell, either in vitro or in vivo. Illustrative vectors include, for example, plasmids, viral vectors, liposomes and other gene delivery vehicles. The polynucleotide to be delivered, sometimes referred to as a “target polynucleotide” or “transgene,” may comprise a coding sequence of interest in gene therapy (such as a gene encoding a protein of therapeutic interest) and/or a selectable or detectable marker.

“AAV” is adeno-associated virus, and may be used to refer to the virus itself or derivatives thereof. The term covers all subtypes, serotypes and pseudotypes, and both naturally occurring and recombinant forms, except where required otherwise. As used herein, the term “serotype” refers to an AAV which is identified by and distinguished from other AAVs based on its binding properties, e.g., there are many serotypes of AAVs, including but not limited to AAV1, AAV2, AAV2tYF, AAV3, AAV4, AAV5, AAV6, AAV7, AAV8, AAV9, AAV10, AAV11, AAVrh10, AAV.rh20, AAV.rh39, AAV.Rh74, and AAV.hu37, and the term encompasses pseudotypes with the same binding properties. Thus, for example, AAV9 serotypes include AAV with the binding properties of AAV9, e.g., a pseudotyped AAV comprising AAV9 capsid and a rAAV genome which is not derived or obtained from AAV9 or which genome is chimeric. The abbreviation “rAAV” refers to recombinant adeno-associated virus, also referred to as a recombinant AAV vector (or “rAAV vector”).

An “AAV virus” refers to a viral particle composed of at least one AAV capsid protein and an encapsidated polynucleotide. If the particle comprises a heterologous polynucleotide (i.e., a polynucleotide other than a wild-type AAV genome such as a transgene to be delivered to a mammalian cell), it is typically referred to as “rAAV”. An AAV “capsid protein” includes a capsid protein of a wild-type AAV, as well as modified forms of an AAV capsid protein which are structurally and or functionally capable of packaging a rAAV genome and bind to at least one specific cellular receptor which may be different than a receptor employed by wild type AAV. A modified AAV capsid protein includes a chimeric AAV capsid protein such as one having amino acid sequences from two or more serotypes of AAV, e.g., a capsid protein formed from a portion of the capsid protein from AAV9 fused or linked to a portion of the capsid protein from AAV-2, and a AAV capsid protein having a tag or other detectable non-AAV capsid peptide or protein fused or linked to the AAV capsid protein, e.g., a portion of an antibody molecule which binds a receptor other than the receptor for AAV9, such as the transferrin receptor, may be recombinantly fused to the AAV9 capsid protein.

A “pseudotyped” rAAV is an infectious virus having any combination of an AAV capsid protein and an AAV genome. Capsid proteins from any AAV serotype may be employed with a rAAV genome which is derived or obtainable from a wild-type AAV genome of a different serotype or which is a chimeric genome, i.e., formed from AAV DNA from two or more different serotypes, e.g., a chimeric genome having 2 inverted terminal repeats (ITRs), each ITR from a different serotype or chimeric ITRs. The use of chimeric genomes such as those comprising ITRs from two AAV serotypes or chimeric ITRs can result in directional recombination which may further enhance the production of transcriptionally active intermolecular concatamers. Thus, the 5′ and 3′ ITRs within a rAAV vector of the disclosure may be homologous, i.e., from the same serotype, heterologous, i.e., from different serotypes, or chimeric, i.e., an ITR which has ITR sequences from more than one AAV serotype.

rAAV Vectors

Adeno-associated viruses of any serotype are suitable to prepare rAAV, since the various serotypes are functionally and structurally related, even at the genetic level. All AAV serotypes apparently exhibit similar replication properties mediated by homologous rep genes; and all generally bear three related capsid proteins such as those expressed in AAV2. The degree of relatedness is further suggested by heteroduplex analysis which reveals extensive cross-hybridization between serotypes along the length of the genome; and the presence of analogous self-annealing segments at the termini that correspond to ITRs. The similar infectivity patterns also suggest that the replication functions in each serotype are under similar regulatory control. Among the various AAV serotypes, AAV2 is most commonly employed.

An AAV vector of the disclosure typically comprises a polynucleotide that is heterologous to AAV. The polynucleotide is typically of interest because of a capacity to provide a function to a target cell in the context of gene therapy, such as up- or down-regulation of the expression of a certain phenotype. Such a heterologous polynucleotide or “transgene,” generally is of sufficient length to provide the desired function or encoding sequence.

Where transcription of the heterologous polynucleotide is desired in the intended target cell, it can be operably linked to its own or to a heterologous promoter, depending for example on the desired level and/or specificity of transcription within the target cell, as is known in the art. Various types of promoters and enhancers are suitable for use in this context. Constitutive promoters provide an ongoing level of gene transcription, and may be preferred when it is desired that the therapeutic or prophylactic polynucleotide be expressed on an ongoing basis. Inducible promoters generally exhibit low activity in the absence of the inducer, and are up-regulated in the presence of the inducer. They may be preferred when expression is desired only at certain times or at certain locations, or when it is desirable to titrate the level of expression using an inducing agent. Promoters and enhancers may also be tissue-specific: that is, they exhibit their activity only in certain cell types, presumably due to gene regulatory elements found uniquely in those cells.

Illustrative examples of promoters are the chicken beta-actin (CB7) promoter with cytomegalovirus (CMV) enhancer, the SV40 late promoter from simian virus 40, the Baculovirus polyhedron enhancer/promoter element, Herpes Simplex Virus thymidine kinase (HSV tk), the immediate early promoter from cytomegalovirus (CMV) and various retroviral promoters including LTR elements. Inducible promoters include heavy metal ion inducible promoters (such as the mouse mammary tumor virus (mMTV) promoter or various growth hormone promoters), and the promoters from T7 phage which are active in the presence of T7 RNA polymerase. By way of illustration, examples of tissue-specific promoters include various surfactin promoters (for expression in the lung), myosin promoters (for expression in muscle), and albumin promoters (for expression in the liver). A large variety of other promoters are known and generally available in the art, and the sequences of many such promoters are available in sequence databases such as the GenBank database.

Where translation is also desired in the intended target cell, the heterologous polynucleotide will preferably also comprise control elements that facilitate translation (such as a ribosome binding site or “RBS” and a polyadenylation signal). Accordingly, the heterologous polynucleotide generally comprises at least one coding region operatively linked to a suitable promoter, and may also comprise, for example, an operatively linked enhancer, ribosome binding site and poly-A signal. The heterologous polynucleotide may comprise one encoding region, or more than one encoding regions under the control of the same or different promoters. The entire unit, containing a combination of control elements and encoding region, is often referred to as an expression cassette.

The heterologous polynucleotide is integrated by recombinant techniques into or in place of the AAV genomic coding region (i.e., in place of the AAV rep and cap genes), but is generally flanked on either side by AAV inverted terminal repeat (ITR) regions. This means that an ITR appears both upstream and downstream from the coding sequence, either in direct juxtaposition, e.g., (although not necessarily) without any intervening sequence of AAV origin in order to reduce the likelihood of recombination that might regenerate a replication-competent AAV genome. However, a single ITR may be sufficient to carry out the functions normally associated with configurations comprising two ITRs (see, for example, WO 94/13788), and vector constructs with only one ITR can thus be employed in conjunction with the packaging and production methods of the present disclosure.

The native promoters for rep are self-regulating, and can limit the amount of AAV particles produced. The rep gene can also be operably linked to a heterologous promoter, whether rep is provided as part of the vector construct, or separately. Any heterologous promoter that is not strongly down-regulated by rep gene expression is suitable; but inducible promoters may be preferred because constitutive expression of the rep gene can have a negative impact on the host cell. A large variety of inducible promoters are known in the art; including, by way of illustration, heavy metal ion inducible promoters (such as metallothionein promoters); steroid hormone inducible promoters (such as the MMTV promoter or growth hormone promoters); and promoters such as those from T7 phage which are active in the presence of T7 RNA polymerase. One sub-class of inducible promoters are those that are induced by the helper virus that is used to complement the replication and packaging of the rAAV vector. A number of helper-virus-inducible promoters have also been described, including the adenovirus early gene promoter which is inducible by adenovirus E1A protein; the adenovirus major late promoter; the herpesvirus promoter which is inducible by herpesvirus proteins such as VP16 or 1CP4; as well as vaccinia or poxvirus inducible promoters.

Methods for identifying and testing helper-virus-inducible promoters have been described (see, e.g., WO 96/17947). Thus, methods are known in the art to determine whether or not candidate promoters are helper-virus-inducible, and whether or not they will be useful in the generation of high efficiency packaging cells. Briefly, one such method involves replacing the p5 promoter of the AAV rep gene with the putative helper-virus-inducible promoter (either known in the art or identified using well-known techniques such as linkage to promoter-less “reporter” genes). The AAV rep-cap genes (with p5 replaced), e.g., linked to a positive selectable marker such as an antibiotic resistance gene, are then stably integrated into a suitable host cell (such as the HeLa or A549 cells exemplified below). Cells that are able to grow relatively well under selection conditions (e.g., in the presence of the antibiotic) are then tested for their ability to express the rep and cap genes upon addition of a helper virus. As an initial test for rep and/or cap expression, cells can be readily screened using immunofluorescence to detect Rep and/or Cap proteins. Confirmation of packaging capabilities and efficiencies can then be determined by functional tests for replication and packaging of incoming rAAV vectors. Using this methodology, a helper-virus-inducible promoter derived from the mouse metallothionein gene has been identified as a suitable replacement for the p5 promoter, and used for producing high titers of rAAV particles (as described in WO 96/17947).

Removal of one or more AAV genes is in any case desirable, to reduce the likelihood of generating replication-competent AAV (“RCA”). Accordingly, encoding or promoter sequences for rep, cap, or both, may be removed, since the functions provided by these genes can be provided in trans, e.g., in a stable line or via co-transfection.

The resultant vector is referred to as being “defective” in these functions. In order to replicate and package the vector, the missing functions are complemented with a packaging gene, or a plurality thereof, which together encode the necessary functions for the various missing rep and/or cap gene products. The packaging genes or gene cassettes are in one embodiment not flanked by AAV ITRs and in one embodiment do not share any substantial homology with the rAAV genome. Thus, in order to minimize homologous recombination during replication between the vector sequence and separately provided packaging genes, it is desirable to avoid overlap of the two polynucleotide sequences. The level of homology and corresponding frequency of recombination increase with increasing length of homologous sequences and with their level of shared identity. The level of homology that will pose a concern in a given system can be determined theoretically and confirmed experimentally, as is known in the art. Typically, however, recombination can be substantially reduced or eliminated if the overlapping sequence is less than about a 25 nucleotide sequence if it is at least 80% identical over its entire length, or less than about a 50 nucleotide sequence if it is at least 70% identical over its entire length. Of course, even lower levels of homology are preferable since they will further reduce the likelihood of recombination. It appears that, even without any overlapping homology, there is some residual frequency of generating RCA. Even further reductions in the frequency of generating RCA (e.g., by nonhomologous recombination) can be obtained by “splitting” the replication and encapsidation functions of AAV, as described by Allen et al., WO 98/27204).

The rAAV vector construct, and the complementary packaging gene constructs can be implemented in this disclosure in a number of different forms. Viral particles, plasmids, and stably transformed host cells can all be used to introduce such constructs into the packaging cell, either transiently or stably.

In certain embodiments of this disclosure, the AAV vector and complementary packaging gene(s), if any, are provided in the form of bacterial plasmids, AAV particles, or any combination thereof. In other embodiments, either the AAV vector sequence, the packaging gene(s), or both, are provided in the form of genetically altered (preferably inheritably altered) eukaryotic cells. The development of host cells inheritably altered to express the AAV vector sequence, AAV packaging genes, or both, provides an established source of the material that is expressed at a reliable level.

A variety of different genetically altered cells can thus be used in the context of this disclosure. By way of illustration, a mammalian host cell may be used with at least one intact copy of a stably integrated rAAV vector. An AAV packaging plasmid comprising at least an AAV rep gene operably linked to a promoter can be used to supply replication functions (as described in U.S. Pat. No. 5,658,776). Alternatively, a stable mammalian cell line with an AAV rep gene operably linked to a promoter can be used to supply replication functions (see, e.g., Trempe et al., WO 95/13392); Burstein et al. (WO 98/23018); and Johnson et al. (U.S. Pat. No. 5,656,785). The AAV cap gene, providing the encapsidation proteins as described above, can be provided together with an AAV rep gene or separately (see, e.g., the above-referenced applications and patents as well as Allen et al. (WO 98/27204). Other combinations are possible and included within the scope of this disclosure.

### AAV Delivery

Any route of rAAV administration may be employed so long as that route and the amount administered are prophylactically or therapeutically useful. In one example, routes of administration to the CNS include intrathecal and intracranial. Intracranial administration may be to the cisterna magna or ventricle. The term “cisterna magna” is intended to include access to the space around and below the cerebellum via the opening between the skull and the top of the spine. The term “cerebral ventricle” is intended to include the cavities in the brain that are continuous with the central canal of the spinal cord. Intracranial administration is via injection or infusion and suitable dose ranges for intracranial, intracisternal or intrathecal administration are generally about 10⁸ to 10¹⁵ genome copies of viral vector per gram brain as determined by magnetic resonance imaging (MRI). The total volume of product administered will not exceed 10% of the total CSF volume, i.e., about 50 mL in and infant brain and about 150 mL in adult brain. For instance, about 10⁸, 10⁹, 10¹⁰, 10¹¹, 10¹², 10¹³, 10¹⁴, 10¹⁵, viral genomes or genome copies of viral vector would be delivered in about 30, 40, 50, 60, 70, 80, 90, 100, 110, 120, 130, 140, 150, 160, 170, 180, 190 or 200 mL. It should be understood that the aforementioned dosage is merely an exemplary dosage and those of skill in the art will understand that this dosage may be varied. Effective doses may be extrapolated from dose-responsive curves derived from in vitro or in vivo test systems.

The AAV delivered in the intrathecal methods of treatment of the present disclosure may be administered through any convenient route commonly used for intrathecal administration. For example, the intrathecal administration may be via a slow infusion of the formulation for about an hour. Intrathecal administration is via injection or infusion and suitable dose ranges for intrathecal administration are generally about 10³ to 10¹⁵ infectious units of viral vector per microliter delivered in, for example, 1, 2, 5, 10, 25, 50, 75, 100, 150, 200, 250 or more milliliters, e.g., 1 to 10,000 milliliters or 0.5 to 15 milliliters, of single injection volume. For instance, viral genomes or infectious units of vector per microliter would generally contain about 10⁴, 10⁵, 10⁶, 10⁷, 10⁸, 10⁹, 10¹⁰, 10¹¹, 10¹², 10¹³, or 10¹⁴ viral genomes or infectious units of viral vector.

The AAV delivered in the methods of treatment may be administered in suitable dose ranges, generally about 10³ to 10¹⁵ infectious units of viral vector per microliter delivered in, for example, 1, 2, 5, 10, 25, 50, 75 or 100 or more milliliters, e.g., 1 to 10,000 milliliters or 0.5 to 15 milliliters. For instance, viral genomes or infectious units of vector per microliter would generally contain about 10⁴, 10⁵, 10⁶, 10⁷, 10⁸, 10⁹, 10¹⁰, 10¹¹, 10¹², 10¹³, 10¹⁴, 10¹⁵, 10¹⁶, or 10¹⁷ viral genomes or infectious units of viral vector, e.g., at least 1.2×10¹¹ genomes or infectious units, for instance at least 2×10¹¹ up to about 2×10¹² genomes or infectious units or about 1×10¹³ to about 5×10¹⁶ genomes or infectious units In one embodiment, the AAV employed for delivery is one that binds to glycans with terminal galactose residues and in one embodiment the dose is 2 to 8 fold higher than 9×10¹⁰ to less than 1×10¹³AAV genomes or infectious units of viral vector.

The AAV delivered in the IT/IC methods disclosed herein may be administered in suitable dose ranges, generally about 1×10⁸ to 1×10¹³ GC/g brain, 1×10⁹ to 1×10¹¹ GC/g brain, or 1×10¹⁰ to 1×10¹² GC/g brain. For example, the AAV delivered in the IT/IC methods disclosed herein may be administered at about 1.3×10¹⁰, 6.5×10¹⁰ or 2×10¹¹ GC/g brain. In one embodiment, the AAV delivered in the IT/IC methods disclosed herein may be administered at about 1×10¹⁰, 1.3×10¹⁰, 5.0×10¹⁰ or 2×10¹¹ GC/g brain. Amounts administered may be based on estimated brain mass, e.g., using screening magnetic resonance imaging (MRI). In one embodiment, the total volume of product administered may not exceed 10% of the total CSF volume (estimated to be −50 mL in infant brain and −150 mL in adult brain).

The AAV delivered in the IV methods disclosed herein may be administered in suitable dose ranges, generally about 1×10⁸ to 1×10¹³ GC/kg, 1×10⁹ to 1×10¹¹ GC/kg, or 1×10¹⁰ to 1×10¹² GC/kg. For example, the AAV delivered in the IV methods disclosed herein may be administered at about 1×10¹¹, 5×10¹¹ or 1×10¹² GC/kg. In one embodiment, the AAV delivered in the IV methods disclosed herein may be administered at about 1×10¹¹ GC/kg, 2×10¹¹ GC/kg, 3×10¹¹ GC/kg, 4×10¹¹ GC/kg, 5×10¹¹ GC/kg, 6×10¹¹ GC/kg, 7×10¹¹ GC/kg, 1×10¹¹ GC/kg, 8×10¹¹ GC/kg, 9×10¹¹ GC/kg, or 1×10¹² GC/kg. The dose volume may be somewhere between 10 mL and 100 mL depending on body weight, e.g., 10 mL to 20 mL, 20 mL to 30 mL, 30 mL to 40 mL, 40 mL to 50 mL, 50 mL to 60 mL, 60 mL to 70 mL, 70 mL to 80 mL or 90 mL to 100 mL.

The CNS therapy may result in the normalization of lysosomal storage granules in the neuronal and/or meningeal tissue of the subjects as discussed above. It is contemplated that the deposition of storage granules is ameliorated from neuronal and glial tissue, thereby alleviating the developmental delay and regression seen in individuals suffering with lysosomal storage disease. Other effects of the therapy may include the normalization of lysosomal storage granules in the cerebral meninges near the arachnoid granulation, the presence of which in lysosomal storage disease result in high pressure hydrocephalus. The methods of the disclosure also may be used in treating spinal cord compression that results from the presence of lysosomal storage granules in the cervical meninges near the cord at C1-C5 or elsewhere in the spinal cord. The methods of the disclosure also are directed to the treatment of cysts that are caused by the perivascular storage of lysosomal storage granules around the vessels of the brain. In other embodiments, the therapy also may advantageously result in normalization of liver volume and urinary glycosaminoglycan excretion, reduction in spleen size and apnea/hypopnea events, increase in height and growth velocity in prepubertal subjects, increase in shoulder flexion and elbow and knee extension, and reduction in tricuspid regurgitation or pulmonic regurgitation.

The intrathecal administration may comprise introducing the composition into the lumbar area. Any such administration may be via a bolus injection. Depending on the severity of the symptoms and the responsiveness of the subject to the therapy, the bolus injection may be administered once per week, once per month, once every 6 months or annually. In other embodiments, the intrathecal administration is achieved by use of an infusion pump. Those of skill in the art are aware of devices that may be used to effect intrathecal administration of a composition. The composition may be intrathecally given, for example, by a single injection, or continuous infusion. It should be understood that the dosage treatment may be in the form of a single dose administration or multiple doses.

As used herein, the term “intrathecal administration” is intended to include delivering a pharmaceutical composition directly into the cerebrospinal fluid of a subject, by techniques including lateral cerebroventricular injection through a burrhole or cistemal or lumbar puncture or the like. The term “lumbar region” is intended to include the area between the third and fourth lumbar (lower back) vertebrae and, more inclusively, the L2-S1 region of the spine.

Other routes of delivery may be employed, e.g., systemic administration such as intravenous administration of rAAV or other viral vector.

Administration of a composition in accordance with the present disclosure to any of the above mentioned sites can be achieved by direct injection of the composition or by the use of infusion pumps. For injection, the composition can be formulated in liquid solutions, e.g., in physiologically compatible buffers such as Hank's solution, Ringer's solution or phosphate buffer. In addition, the enzyme may be formulated in solid form and re-dissolved or suspended immediately prior to use. Lyophilized forms are also included. The injection can be, for example, in the form of a bolus injection or continuous infusion (e.g., using infusion pumps) of the enzyme.

In one embodiment of the disclosure, the rAAV is administered by lateral cerebroventricular injection into the brain of a subject. The injection can be made, for example, through a burr hole made in the subject's skull. In another embodiment, the enzyme and/or other pharmaceutical formulation is administered through a surgically inserted shunt into the cerebral ventricle of a subject. For example, the injection can be made into the lateral ventricles, which are larger, even though injection into the third and fourth smaller ventricles can also be made. In yet another embodiment, the compositions used in the present disclosure are administered by injection into the cisterna magna or lumbar area of a subject.

In one embodiment, an immune suppressant or immunotolerizing agent may be administered by any route including parenterally. In one embodiment, the immune suppressant or immunotolerizing agent may be administered by subcutaneous, intramuscular, or intravenous injection, orally, intrathecally, or intracranially, or by sustained release, e.g., using a subcutaneous implant. The immune suppressant or immunotolerizing agent may be dissolved or dispersed in a liquid carrier vehicle. For parenteral administration, the active material may be suitably admixed with an acceptable vehicle, e.g., of the vegetable oil variety such as peanut oil, cottonseed oil and the like. Other parenteral vehicles such as organic compositions using solketal, glycerol, formal, and aqueous parenteral formulations may also be used. For parenteral application by injection, compositions may comprise an aqueous solution of a water soluble pharmaceutically acceptable salt of the active acids according to the disclosure, desirably in a concentration of 0.01-10%, and optionally also a stabilizing agent and/or buffer substances in aqueous solution. Dosage units of the solution may advantageously be enclosed in ampules.

The composition, e.g., rAAV containing composition, immune suppressant containing composition or immunotolerizing composition, may be in the form of an injectable unit dose. Examples of carriers or diluents usable for preparing such injectable doses include diluents such as water, ethyl alcohol, macrogol, propylene glycol, ethoxylated isostearyl alcohol, polyoxyisostearyl alcohol and polyoxyethylene sorbitan fatty acid esters, pH adjusting agents or buffers such as sodium citrate, sodium acetate and sodium phosphate, stabilizers such as sodium pyrosulfite, EDTA, thioglycolic acid and thiolactic acid, isotonic agents such as sodium chloride and glucose, local anesthetics such as procaine hydrochloride and lidocaine hydrochloride. Furthermore, usual solubilizing agents and analgesics may be added. Injections can be prepared by adding such carriers to the enzyme or other active, following procedures well known to those of skill in the art. A thorough discussion of pharmaceutically acceptable excipients is available in REMINGTON'S PHARMACEUTICAL SCIENCES (Mack Pub. Co., N.J. 1991). The pharmaceutically acceptable formulations can easily be suspended in aqueous vehicles and introduced through conventional hypodermic needles or using infusion pumps. Prior to introduction, the formulations can be sterilized with, preferably, gamma radiation or electron beam sterilization.

When the immune suppressant or immunotolerizing agent is administered in the form of a subcutaneous implant, the compound is suspended or dissolved in a slowly dispersed material known to those skilled in the art, or administered in a device which slowly releases the active material through the use of a constant driving force such as an osmotic pump. In such cases, administration over an extended period of time is possible.

The dosage at which the immune suppressant or immunotolerizing agent containing composition is administered may vary within a wide range and will depend on various factors such as the severity of the disease, the age of the patient, etc., and may have to be individually adjusted. A possible range for the amount which may be administered per day is about 0.1 mg to about 2000 mg or about 1 mg to about 2000 mg. The compositions containing the immune suppressant or immunotolerizing agent may suitably be formulated so that they provide doses within these ranges, either as single dosage units or as multiple dosage units. In addition to containing an immune suppressant, the subject formulations may contain one or more rAAV encoding a therapeutic gene product.

Compositions described herein may be employed in combination with another medicament. The compositions can appear in conventional forms, for example, aerosols, solutions, suspensions, or topical applications, or in lyophilized form.

Typical compositions include a rAAV, and optionally an immune suppressant, a permeation enhancer, or a combination thereof, and a pharmaceutically acceptable excipient which can be a carrier or a diluent. For example, the active agent(s) may be mixed with a carrier, or diluted by a carrier, or enclosed within a carrier. When the active agent is mixed with a carrier, or when the carrier serves as a diluent, it can be solid, semi-solid, or liquid material that acts as a vehicle, excipient, or medium for the active agent. Some examples of suitable carriers are water, salt solutions, alcohols, polyethylene glycols, polyhydroxyethoxylated castor oil, peanut oil, olive oil, gelatin, lactose, terra alba, sucrose, dextrin, magnesium carbonate, sugar, cyclodextrin, amylose, magnesium stearate, talc, gelatin, agar, pectin, acacia, stearic acid or lower alkyl ethers of cellulose, silicic acid, fatty acids, fatty acid amines, fatty acid monoglycerides and diglycerides, pentaerythritol fatty acid esters, polyoxyethylene, hydroxymethylcellulose and polyvinylpyrrolidone. Similarly, the carrier or diluent can include any sustained release material known in the art, such as glyceryl monostearate or glyceryl distearate, alone or mixed with a wax.

The formulations can be mixed with auxiliary agents which do not deleteriously react with the active agent(s). Such additives can include wetting agents, emulsifying and suspending agents, salt for influencing osmotic pressure, buffers and/or coloring substances preserving agents, sweetening agents or flavoring agents. The compositions can also be sterilized if desired.

If a liquid carrier is used, the preparation can be in the form of a liquid such as an aqueous liquid suspension or solution. Acceptable solvents or vehicles include sterilized water, Ringer's solution, or an isotonic aqueous saline solution.

The agent(s) may be provided as a powder suitable for reconstitution with an appropriate solution as described above. Examples of these include, but are not limited to, freeze dried, rotary dried or spray dried powders, amorphous powders, granules, precipitates, or particulates. The composition can optionally contain stabilizers, pH modifiers, surfactants, bioavailability modifiers and combinations of these. A unit dosage form can be in individual containers or in multi-dose containers.

Compositions contemplated by the present disclosure may include, for example, micelles or liposomes, or some other encapsulated form, or can be administered in an extended release form to provide a prolonged storage and/or delivery effect, e.g., using biodegradable polymers, e.g., polylactide-polyglycolide. Examples of other biodegradable polymers include poly(orthoesters) and poly(anhydrides).

Polymeric nanoparticles, e.g., comprised of a hydrophobic core of polylactic acid (PLA) and a hydrophilic shell of methoxy-poly(ethylene glycol) (MPEG), may have improved solubility and targeting to the CNS. Regional differences in targeting between the microemulsion and nanoparticle formulations may be due to differences in particle size.

Liposomes are very simple structures consisting of one or more lipid bilayers of amphiphilic lipids, i.e., phospholipids or cholesterol. The lipophilic moiety of the bilayers is turned towards each other and creates an inner hydrophobic environment in the membrane. Liposomes are suitable drug carriers for some lipophilic drugs which can be associated with the non-polar parts of lipid bilayers if they fit in size and geometry. The size of liposomes varies from 20 nm to few μm.

Mixed micelles are efficient detergent structures which are composed of bile salts, phospholipids, tri, di- and monoglycerides, fatty acids, free cholesterol and fat soluble micronutrients. As long-chain phospholipids are known to form bilayers when dispersed in water, the preferred phase of short chain analogues is the spherical micellar phase. A micellar solution is a thermodynamically stable system formed spontaneously in water and organic solvents. The interaction between micelles and hydrophobic/lipophilic drugs leads to the formation of mixed micelles (MM), often called swollen micelles, too. In the human body, they incorporate hydrophobic compounds with low aqueous solubility and act as a reservoir for products of digestion, e.g. monoglycerides.

Lipid microparticles includes lipid nano- and microspheres. Microspheres are generally defined as small spherical particles made of any material which are sized from about 0.2 to 100 μm. Smaller spheres below 200 nm are usually called nanospheres. Lipid microspheres are homogeneous oil/water microemulsions similar to commercially available fat emulsions, and are prepared by an intensive sonication procedure or high pressure emulsifying methods (grinding methods). The natural surfactant lecithin lowers the surface tension of the liquid, thus acting as an emulsifier to form a stable emulsion. The structure and composition of lipid nanospheres is similar to those of lipid microspheres, but with a smaller diameter.

Polymeric nanoparticles serve as carriers for a broad variety of ingredients. The active components may be either dissolved in the polymetric matrix or entrapped or adsorbed onto the particle surface. Polymers suitable for the preparation of organic nanoparticles include cellulose derivatives and polyesters such as poly(lactic acid), poly(glycolic acid) and their copolymer. Due to their small size, their large surface area/volume ratio and the possibility of functionalization of the interface, polymeric nanoparticles are ideal carrier and release systems. If the particle size is below 50 nm, they are no longer recognized as particles by many biological and also synthetic barrier layers, but act similar to molecularly disperse systems.

Thus, the composition of the disclosure can be formulated to provide quick, sustained, controlled, or delayed release, or any combination thereof, of the active agent after administration to the individual by employing procedures well known in the art. In one embodiment, the enzyme is in an isotonic or hypotonic solution. In one embodiment, for enzymes that are not water soluble, a lipid-based delivery vehicle may be employed, e.g., a microemulsion such as that described in WO 2008/049588, the disclosure of which is incorporated by reference herein, or liposomes.

In one embodiment, the preparation can contain an agent, dissolved or suspended in a liquid carrier, such as an aqueous carrier, for aerosol application. The carrier can contain additives such as solubilizing agents, e.g., propylene glycol, surfactants, absorption enhancers such as lecithin (phosphatidylcholine) or cyclodextrin, or preservatives such as parabens. For example, in addition to solubility, efficient delivery to the CNS following administration may be dependent on membrane permeability.

Generally, the active agents are dispensed in unit dosage form including the active ingredient together with a pharmaceutically acceptable carrier per unit dosage. Usually, dosage forms suitable for administration include from about 125 μg to about 125 mg, e.g., from about 250 μg to about 50 mg, or from about 2.5 mg to about 25 mg, of the compounds admixed with a pharmaceutically acceptable carrier or diluent.

Dosage forms can be administered daily, or more than once a day, such as twice or thrice daily. Alternatively, dosage forms can be administered less frequently than daily, such as every other day, or weekly, if found to be advisable by a prescribing physician.

### Example I

### AAV Vector-Mediated Iduronidase Gene Delivery in a Murine Model of Mucopolysaccharidosis Type I: Comparing Different Routes of Delivery to the CNS

Mucopolysaccharidosis type I (MPS 1) is an inherited metabolic disorder caused by deficiency of the lysosomal enzyme alpha-L-iduronidase (IDUA). Systemic and abnormal accumulation of glycosaminoglycans is associated with growth delay, organomegaly, skeletal dysplasia, and cardiopulmonary disease. Individuals with the most severe form of the disease (Hurler syndrome) suffer from neurodegeneration, mental retardation, and early death. The two current treatments for MPS I (hematopoietic stem cell transplantation and enzyme replacement therapy) cannot effectively treat all central nervous system (CNS) manifestations of the disease.

With respect to gene therapy, it was previously demonstrated that intravascular delivery of AAV9 in adult mice does not achieve widespread direct neuronal targeting (see Foust et al, 2009). Previous work also showed that direct injection of AAV8-IDUA into the CNS of adult IDUA-deficient mice resulted in a low frequency or a poor level of transgene expression. The following examples, which use a pre-clinical model for the treatment of MPS1, surprisingly demonstrate that direct injection of AAV9-IDUA into the CNS of immunocompetent adult IDUA-deficient mice resulted in IDUA enzyme expression and activity that is the same or higher than IDUA enzyme expression and activity in wild-type adult mice.

### Methods

AAV9-IDUA preparation. The AAV-IDUA vector construct (MCI) has been previously described (Wolf et al., 2011) (mCags promoter). AAV-IDUA plasmid DNA was packaged into AAV9 virions at the University of Florida Vector Core, yielding a titer of 3×10¹³ vector genomes per milliliter.

ICV infusions. Adult Idua−/− mice were anesthetized using a cocktail of ketamine and xylazine (100 mg ketamine+10 mg xylazine per kg) and placed on a stereotactic frame. Ten microliters of AAV9-IDUA were infused into the right-side lateral ventricle (stereotactic coordinates AP 0.4, ML 0.8, DV 2.4 mm from bregma) using a Hamilton syringe. The animals were returned to their cages on heating pads for recovery.

Intrathecal infusions. Infusions into young adult mice were carried out by injection of 10 μL AAV vector containing solution between the L5 and L6 vertebrae 20 minutes after intravenous injection of 0.2 mL 25% mannitol.

Immunotolerization. Newborn IDUA deficient mice were injected through the facial temporal vein with 5 μL containing 5.8 μg of recombinant iduronidase protein (Aldurazyme), and then the animals were returned to their cage.

Cyclophosphamide immunosuppression. For immunosuppression, animals were administered cyclophosphamide once per week at a dose of 120 mg/kg starting one day after infusion with AAV9-IDUA vector.

Animals. Animals were anesthetized with ketamine/xylazine (100 mg ketamine+10 mg xylazine per kg) and transcardially perfused with 70 mL PBS prior to sacrifice. Brains were harvested and microdissected on ice into cerebellum, hippocampus, striatum, cortex, and brainstem/thalamus (“rest”). The samples were frozen on dry ice and then stored at −80° C. Samples were thawed and homogenized in 1 mL of PBS using a motorized pestle and permeabilized with 0.1% Triton X-100. IDUA activity was determined by fluorometric assay using 4MU-iduronide as the substrate. Activity is expressed in units (percent substrate converted to product per minute) per mg protein as determined by Bradford assay (BioRad).

Tissues. Tissue homogenates were clarified by centrifugation for 3 minutes at 13,000 rpm using an Eppendorf tabletop centrifuge model 5415D (Eppendorf and incubated overnight with proteinase K, DNase1, and Rnase. GAG concentration was determined using the Blyscan Sulfated Glycosaminoglycan Assay (Accurate Chemical) according to the manufacturer's instructions.

### Results

Iduronidase-deficient mice were administered AAV either intracerebroventricularly (ICV) or intrathecally (IT). To prevent immune response, animals were either immunosuppressed with cyclophosphamide (CP), immunotolerized at birth by intravenous administration of human iduonidase protein (aldurazyme), or the injections were carried out in NOD-SCID immunodeficient mice that were also iduronidase deficient. Animals were sacrificed at the indicated time post-treatment, the brains were microdissected and extracts assayed for iduronidase activity.

Immunodeficient, IDUA deficient animals that were injected ICV with AAV-IDUA vector exhibited high levels of IDUA expression (10 to 100 times wild type) in all areas of the brain, with the highest level observed in the brain stem and thalamus (“rest”).

Immunosuppressed animals administered AAV vector by ICV route had a relatively lower level of enzyme in the brain compared to the immunodeficent animals. Note that immunosuppression may have been compromised in these animals because CP was withdrawn 2 weeks before sacrifice due to poor health.

Immunosuppressed animals were administered AAV vector by the IT route. Immunotolerized animals administered AAV vector ICV exhibited widespread IDUA activity in all parts of the brain, similar to that observed in the immunodeficient animals, indicating the effectiveness of the immunotolerization procedure.

GAG storage material was assayed in the different sections of the brain for all four of the test groups. For each group, the mean of each portion of the brain is shown on the left, the values for each of the individual animals is shown on the right. IDUA deficient animals (far left) contained high levels of GAG compared to wild type animals (magenta bar). GAG levels were at wild-type or lower than wild type for all portions of the brain in all groups of AAV-treated animals. GAG levels were slightly although not significantly higher than wild-type in cortex and brainstem of animals administered AAV9-IDUA intrathecally.

### Conclusions

The results show high and widespread distribution of IDUA in the brain regardless of the route of delivery (ICV or IT) although IDUA expression in striatum and hippocampus was lower in animals injected IT versus ICV. There appears to be an immune response since immune deficient mice have higher levels of expression than immunocompetent mice. With regard to ICV injection, when CP was withdrawn early, IDUA expression is lower. In addition, immunotolerization was effective in restoring high levels of enzyme activity. Further, GAG levels were restored to normal in all treated experimental groups of mice.

### Example II

### Methods

AAV9-IDUA Preparation. AAV-IDUA plasmid was packaged into AAV9 virions at either the University of Florida vector core, or the University of Pennsylvania vector core, yielding a titer of 1-3×10¹³ vector genomes per milliliter.

ICV infusions. See Example I.

Intrathecal infusions. See Example I.

Immunotolerization. As in Example I except: for multiple tolerizations, newborn IDUA deficient mice were injected with the first dose of Aldurazyme in the facial temporal vein, followed by 6 weekly injections administered intraperitoneally.

Cyclophosphamide immunosuppression. See Example I.

Animals. Animals were anesthetized with ketamine/xylazine (100 mg ketamine+10 mg xylazine per kg) and transcardially perfused with 70 mL PBS prior to sacrifice. Brains were harvested and microdissected on ice into cerebellum, hippocampus, striatum, cortex, and brainstem/thalamus (“rest”). The samples were frozen on dry ice and then stored at −80° C.

Tissue IDUA activity. Tissue samples were thawed and homogenized in saline in a tissue homogenizer. Tissue homogenates were clarified by centrifugation at 15,000 rpm in a benchtop Eppendorf centrifuge at 4° C. for 15 minutes. Tissue lysates (supernatant) were collected and analyzed for IDUA activity and GAG storage levels.

Tissue GAG levels. Tissue lysates were incubated overnight with Proteinase K, RNase and DNase. GAG levels were analyzed using the Blyscan Sulfated Glycosaminoglycan Assay according to the manufacturer's instructions.

IDUA Vector copies. Tissue homogenates were used for DNA isolation and subsequent QPCR, as described in Wolf et al. (2011).

### Results

Animals were administered AAV9-IDUA vector either by intracerebroventricular (ICV) or intrathecal (IT) infusion. Vector administration was carried out in NOD-SCID immunodeficient (ID) mice that were also IDUA deficient, or in IDUA deficient mice that were either immunosuppressed with cyclophosphamide (CP), or immunotolerized at birth by a single or multiple injections of human iduronidase protein (Aldurazyme). All vector administrations were carried out in adult animals ranging in age from 3-4.5 months. Animals were injected with 10 μL of vector at a dose of 3×10¹¹ vector genomes per 10 microliters.

IDUA enzyme activities in intracranially infused, immunodeficient, IDUA deficient mice were high in all areas of the brain, ranging from 30- to 300-fold higher than wild type levels. Highest enzyme expressions were seen in thalamus and brain stem, and in the hippocampus.

Animals that were injected intracranially and immunosuppressed with cyclophosphamide (CP) demonstrated significantly lower levels of enzyme activity than other groups. However, CP administration in this case had to be withdrawn 2 weeks prior to sacrifice due to poor health of the animals.

IDUA enzyme levels in animals tolerized at birth with IDUA protein (Aldurazyme) and administered vector intracranially were high in all parts of the brain that ranged from 10- to 1000-fold higher than wild type levels, similar to levels achieved in immunodeficient animals, indicating the effectiveness of the immunotolerization procedure.

IDUA enzyme levels in mice that were injected intrathecally and administered CP on a weekly basis were elevated and were observed in all parts of the brain, especially in the cerebellum and the spinal cord. Levels of enzyme were the lowest in the striatum and hippocampus with activities at wild type levels.

IDUA deficient mice were tolerized with Aldurazyme as described, and injected with vector intrathecally. There was widespread IDUA enzyme activity in all parts of the brain, with highest levels of activity in the brain stem and thalamus, olfactory bulb, spinal cord and the cerebellum. Similarly, the lowest levels of enzyme activity were seen in the striatum, cortex and hippocampus.

Control immunocompetent IDUA deficient animals were infused with vector intrathecally, without immunosuppression or immunotolerization. The results indicate that although enzyme activities were at wild type levels or slightly higher, they are significantly lower than what was observed in animals that underwent immunomodulation. The decreases in enzyme levels were especially significant in the cerebellum, olfactory bulb and thalamus and brain stem, areas that expressed the highest levels of enzyme in immunomodulated animals.

Animals were assayed for GAG storage material. All groups demonstrated clearance of GAG storage, with GAG levels similar to that observed in wild type animals. Animals that were immunosuppressed and injected with AAV9-IDUA vector intrathecally had GAG levels in the cortex that were slightly higher than wild type, but still much lower than untreated IDUA deficient mice.

The presence of AAV9-IDUA vector in animals that were immunotolerized and injected with vector either intracranially or intrathecally was evaluated by QPCR. IDUA copies per cell were higher in animals infused intracranially in comparison with animals infused intrathecally, which is consistent with the higher level of enzyme activity seen in animals injected intracranially.

### Conclusions

High, widespread, and therapeutic levels of IDUA were observed in all areas of the brain after intracerebroventricular and intrathecal routes of AAV9-IDUA administration in adult mice. Enzyme activities were restored to wild type levels or slightly higher in immunocompetent IDUA deficient animals infused with AAV-IDUA intrathecally. Significantly higher levels of IDUA enzyme were observed for both routes of vector injection in animals immunotolerized starting at birth by administration of IDUA protein.

### Example III

Mucopolysaccharidosis type II (MPS II; Hunter Syndrome) is an X-linked recessive inherited lysosomal storage disease caused by deficiency of iduronate-2-sulfatase (IDS) and subsequent accumulation of glycosaminoglycans (GAGs) dermatan and heparan sulphate. Affected individuals exhibit a range in severity of manifestations physically, neurologically, and shortened life expectancy. For example, affected individuals exhibit a range in severity of manifestations such as organomegaly, skeletal dysplasias, cardiopulmonary obstruction, neurocognitive deficit, and shortened life expectancy. There is no cure for MPS II at the moment. Current standard of care is enzyme replacement therapy (ELAPSRASE; idursulfase), which is used to manage disease progression. However, enzyme replacement therapy (ERT) does not result in neurologic improvement. As hematopoetic stem cell transplantation (HSCT) has not shown neurologic benefit for MPS II, there is currently no clinical recourse for patients exhibiting neurologic manifestations of this disease, and new therapies are desperately needed.

AAV9 vectors are developed for delivery of the human IDS coding sequence (AAV9-hIDS) into the central nervous system of MPS II mice to restore IDS levels in the brain and prevent the emergence of neurocognitive deficits in the treated animals. In particular, a series of vectors were generated that encode human IDS with or without the human sulfatase modifying factor-1 (SUMF-1), required for activation of the sulfatase active site. Three routes of administration were used in these experiments: Intrathecal (IT). Intracerebroventricular (ICV) and Intravenous (IV). No significant difference in the enzyme level was found between mice that were treated with AAV9 vector transducing hIDS alone and mice that were treated with AAV9 vector encoding human IDS and SUMF-1, regardless of the route of administration. IT-administrated NOD.SCID (IDS Y+) and C57BL/6 (IDS Y+) did not show elevated IDS activity in the brain and spinal cord when compared to untreated animals, while plasma showed ten-fold higher (NOD.SCID) and 150-fold higher (C57BL/6) levels than untreated animals. IDS-deficient mice intravenously administered AAV9-hIDS exhibited IDS activities in all organs that were comparable to wild type. Moreover, the plasma of IV injected animals showed enzyme activity that was 100-fold higher than wild type. IDS-deficient mice administered AAV9-hIDUA ICV showed IDS activities comparable to wild type in most areas of the brain and peripheral tissues, while some portions of the brain showed two- to four-fold higher activity than wild type. Furthermore, IDS activity in plasma was 200-fold higher than wild type. Surprisingly, IDS enzyme activity in the plasma of all treated animals showed persistence for at least 12 weeks post injection; therefore, IDS enzyme was not immunogenic at least on the C57BL/6 murine background. Additional neurobehavioral testing was conducted using the Barnes maze to differentiate neurocognitive deficits of untreated MPS II animals from that of wild type littermates. It was found that the learning capability of affected animals is distinctively slower than that observed in littermates. Thus, Barnes maze is used to address the benefit of these therapies in the MPS II murine model. These results indicate potential of therapeutic benefit of AAV9 mediated human IDS gene transfer to the CNS to prevent neurologic deficiency in MPS II.

In summary, intracerebroventricular (ICV) injection of AAV9-hIDS resulted in systemic correction of IDS enzyme deficiency, including wild-type levels of IDS in the brain. Co-delivery of hIDS with hSUMF-1 did not increase IDS activity in tissues. hIDS expression was non-immunogenic in WT and MPS II C57BL/6 mice.

The following provides further details in this regard.

Mucopolysaccharidosis type II (MPS II, Hunter syndrome) is a rare x-linked recessive lysosomal disorder caused by defective Iduronate-2-sulfatase (IDS) resulting in accumulation of heparan sulfate and dermatan sulfate glycosaminoglycans (GAGs). Enzyme replacement is the only FDA-approved therapy available for MPS II, but it is expensive and does not improve neurologic outcomes in MPS II patients. As described below, this study evaluated the effectiveness of IDS-encoding adeno-associated virus (AAV) vector encoding human IDS delivered intracerebroventricularly in a murine model of MPS II. Supraphysiological levels of IDS were observed in the circulation (160-fold higher than wild type) for at least 28 weeks post-injection and in most tested peripheral organs (up to 270-fold) at 10 months postinjection. In contrast, only low levels of IDS were observed (7% to 40% of wild type) in all areas of the brain. Sustained IDS expression had a profound effect on normalization of GAG in all tested tissues and on prevention of hepatomegaly. Additionally, sustained IDS expression in the CNS had a prominent effect in preventing neurocognitive deficit in MPS II mice treated at two months of age. The present study demonstrates that CNS-directed, AAV9 mediated gene transfer is a potentially effective treatment for Hunter syndrome as well as other monogenic disorders with neurologic involvement.

### Introduction

The mucopolysaccharidoses (MPSs) are a group of lysosomal disorders caused by deficiency of any one of 11 lysosomal hydrolase that catalyze the breakdown of glycosaminoglycans (GAGs). MPS type II (MPS II; Hunter syndrome), is an X-linked recessive caused by deficiency of iduronate-2-sulfatase (IDS) with subsequent accumulation of substrate (GAGs) in tissues of affected individuals associated with hepatosplenomegaly, skeletal dysplasia, joint stiffness, and airway obstruction. In severe cases, affected individuals exhibit neurocognitive deficits and succumb to the illness in adolescence. The current and only treatment available for MPS II is enzyme replacement therapy (ERT), which is used to mitigate disease progression but without neurologic improvement. Hematopoietic stem cell transplantation, which has been shown to provide long-term benefits for MPS I (Whitley et al., 1993), has not been reported to ameliorate neurodegenerative disease in severe cases of MPS II (McKinnis et al. 1996; Vellodi et al. 2015; Hoogerbrugge et al. 1995).

The Sleeping Beauty (SB) transposon system and minicircles are two non-viral gene therapy platforms that have been successfully used in mice for systemic diseases such as MPS type I and type VII (Aronovich et al. 2009; Aronovich et al. 2007; Osborn et al. 2011). Despite being efficient and providing sustained expression in vivo (Aronovich et al. 2007; Chen 2003), the major drawback of these systems is the inability to penetrate the BBB (Aronovich and Hackett 2015) which has not yet been resolved. This limits the effectiveness of non-viral gene therapy systems for the CNS.

Various viral vectors have been extensively studied in gene therapy clinical trials for many diseases because their potency and sustained expression (Kaufmann et al. 2013). Amongst these vehicles, adeno-associated viral vectors (AAVs) have been shown to be promising candidates for clinical trials in mediating gene transfer for monogenic disorders (Tanaka et al. 2012; Bennett et al. 2012; Nathwani et al. 2014). Unlike other AAV serotypes, adeno-associated viral vector serotype 9 (AAV9) has been demonstrated in many animal models to not only efficiently transduce the CNS and peripheral nervous tissues (PNS), but also penetrate the BBB and transduce various cell types in peripheral tissues (Duque et al., 2009; Foust et al., 2009; Huda et al., 2014; Schuster et al., 2014). Thus AA9 outperforms other viral vectors as a candidate for systemic correction including CNS for monogenic disorders such as MPS II. Herein is reported the effectiveness of CNS-directed, AAV9 mediated human IDS gene transfer to correct IDS deficiency and prevent neurocognitive impairment in a murine model of MPS II.

### Materials and Methods

AAV vector assembly and packaging. All vectors were constructed, packaged, and purified at the Penn Vector Core (Philadelphia, PA) and provided by REGENXBIO Inc. (Rockville, MD). In brief, the expression cassettes contained a chicken beta-actin (CB7) promoter with cytomegalovirus (CMV) enhancer followed by hIDS or human sulfatase modifying factor 1 (hSUMF1), rabbit beta-actin polyadenylation signal on the backbone of AAV2 inverted terminal repeats (ITR) on both 3′- and 5′-ends. Co-expression constructs included an internal ribosome entry site (IRES) positioned between IDS and SUMF1 to initiate translation of SUMF1 downstream of the IRES. In this study, five different vector constructs were investigated: AAV9 expressing human IDS alone (AAV9.hIDS); AAV9 expressing codon-optimized human IDS (AAV9.hIDSco); AAV9 coexpressing human IDS and human SUMF1 (AAV9.hIDS-hSUMF1); AAV9 coexpressing codon-optimized human IDS and codon-optimized human SUMF1 (AAV9.hIDScohSUMF1co); AAV9 expressing human SUMF1 alone (AAV9.hSUMF1). AAV vectors were packaged by co-transfecting 3 plasmids: AAV cis, AAV trans (pAAV2/9 rep and cap), and adenovirus helper (pAdAF6), into HEK 293 cells (Lock et al. 2010). AAV vector was then purified from supernatants using a Profile II depth filter and concentrated by tangential flow filtration (TFF). The concentrated feed stock was reclarified by iodixanol gradient centrifugation, then reconcentrated using a TFF cassette with a 100-kDa MWCO HyStream screen channel membrane. The purified vector was then tested for purity by SDS-PAGE and for potency by qPCR (Lock et al. 2010).

Animal care and husbandry. All animal care and experimental procedures were conducted under approval of the Institutional Animal Care and use Committee (IACUC) of the University of Minnesota. NOD.SCID mice were purchased from The Jackson Laboratory and C57BL/6 wild-type mice were purchased from National Cancer Institute. C57BL6 iduronate-2-sulphatase knockout (IDS KO) mice were kindly provided by Dr. Joseph Muenzer (University of North Carolina, NC, USA) and maintained under specific pathogen-free conditions at the Research Animal Resources (RAR) facilities of the University of Minnesota. MPS II male pups (IDS-0) were generated by breeding heterozygous (IDS+/−) females to wild type (IDS+/0) C57BL/6 males. All pups were genotyped by PCR.

AAV vector administration. For intrathecal injections, eight-week old mice were injected with a dose of 5.6×10¹⁰ vector genomes (vg) of AAV9 vector between the L5 and L6 vertebrae. The injection was performed in conscious animals in a 10-15 second duration. For intravenous injections animals were briefly restrained and injected via tail-vain with a dose of 5.6×10¹⁰ vg. Intracerebroventricular injections were carried out in adult 8-week old mice.

Briefly, animals were injected intraperitoneally with 6 μl of ketamine/xylazine mixture (36 mg/ml ketamine, 5.5 mg/mL xylazine) to produce deep anesthesia and then mounted in a stereotactic frame (Kopf Model 900). An incision was made to expose the cranium, small hole was drilled as a site for the injection, and then a Hamilton syringe (Model 701) was used to carry out the infusion at a rate approximately 0.5 μL per minute by hand. The syringe was left in place for an additional 3 minutes, then slowly withdrawn over a period of at least 2 minutes. The scalp was sutured after completion of the injection, and after recovery from the anesthesia the mouse was then returned to standard housing. All of the mice received a 3-day course of Ketoprofen 2.5 mg/kg subcutaneously and Baytril 5 mg/kg intraperitoneally to prevent infection and inflammation post-surgery.

Sample collection and preparation. Blood was collected by submandibular puncture using sterile 5 mm lancets (Goldenrode™) into Microvette® heparinized coated tubes (SARSTEDT AG & Co.) and centrifuged in an Eppendorf centrifuge 5415D at 7000 rpm for 10 minutes. Plasma was collected and stored at −20° C. to −80° C. for IDS assay. Urine was collected and stored at −20° C. until used for creatinine and GAG assay. Organs were harvested by first determining animal weight using an OHAUS® CS 200 scale before they were euthanized using a CO₂ fume chamber at 2 liter/min for 3 minutes. The animals were perfused with 60 mL of 1×PBS in a 60 ml syringe (BD) with a SURFLO® winged infusion set (TERUMO®) size 23G×.″ by hand-pressure. Heart, lung, liver, spleen, kidney, and spinal cord were harvested. The harvested peripheral organs were weighed using a Sartorius BP 61S scale. Brain was micro-dissected into left and right cerebellum, cortex, hippocampus, striatum, olfactory bulb, and thalamus/brainstem. The organs were immediately snap frozen and stored at −70° C. until further tissue processing.

For tissue processing, cerebellum, hippocampus, striatum, and olfactory bulb were added into pre-assigned 1.5 mL locked-cap microtubes (EPPENDORF) containing 1 scoop (0.2 g/scoop) of 0.5 mm glass beads (NEXT ADVANCE) in 250 μl sterile saline solution. Thalamus/brainstem, cortex and spinal cord were added into assigned locked-cap microtubes containing 2 scoops of 0.5 mm glass beads in 400 μl of sterile saline solution. Half of the lung and the whole spleen were added into the assigned tubes containing 2 scoops of 0.9-2.0 mm stainless steel blend (0.6 g/scoop) in 400 μl of saline solution. Heart, ˜0.3 g liver, and one kidney were added into assigned tubes containing 3 scoops (a mixture of 2 scoops of 0.9-2.0 mm stainless steel blend (0.6 g/scoop) and 1 scoop of 3.2 mm stainless steel beads (0.7 g/scoop)) in 600 μL sterile saline solution. All of the prepared samples in the bead tubes were then homogenized using a Bullet Blender® STORM bead mill homogenizer (NEXT ADVANCE) at speed 12 for 5 minutes to generate tissue homogenate. Fifty-microliter tissue homogenates were transferred into 1.5 microtubes (GeneMate) and stored at −20° C. to −80° C. for quantitative real-time PCR (qPCR). The remaining tissue homogenates were clarified using an Eppendorf centrifuge 5424R at 13,000 rpm for 15 minutes at 4° C. All of the supernatants (Tissue lysates) were transferred into new microtubes and stored at −20° C. to −80° C. until used for IDS, GAG and protein assays.

Iduronate sulfatase assay. IDS enzyme activity was measured in tissue lysates using 4-methylumbelliferyl-α-L-iduronide-2-sulphate disodium (4-MU-aldoA-2S: Toronto Research Chemical Incorporation, Cat. #M334715) as substrate in a two-step assay. Tissue lysates were mixed with 1.25 mM MU-aldoA-2S (in 0.1 M sodium acetate buffer pH 5.0+10 mM lead acetate+0.02% sodium azide) and incubated at 37° C. for 1.5 hours. The first-step reaction was terminated with PiCi buffer to stop IDS enzyme activity (0.2 M Na₂HPO₄/0.1 M citric-acid buffer, pH 4.5+0.02% Na-azide). A final concentration of 1 μg/ml Iduronidase (IDUA: R&D Systems, Cat. #4119-GH-010) was added into the tubes to start the second-step reaction. The tubes were incubated overnight at 37° C. to cleave 4-MU-IdoA into 4-MU. The second-step reaction was terminated by adding 200 μl stop buffer (0.5 M Na₂CO₃+0.5 M NaHCO₃, 0.025% Triton X-100, pH 10.7). The tubes were centrifuged using an Eppendorf centrifuge 5415D at 13,000 rpm for 1 minute. Supernatants were transferred into a round bottom black 96-well plate and fluorescence measured at excitation 365 nm and emission 450 nm, 75 sensitivity using a Synergy MX plate reader and spectrophotometer (Bio Tek) with Gen5 plate reader program. Enzyme activity is expressed in nmol/hr/ml plasma for plasma samples and in nmol/hr/mg protein for tissue extracts. Protein was determined using the Pierce™ 660 nm Protein Assay Reagent with BSA as a standard (CAT. #23208; Thermo Scientific, MN).

Glycosaminoglycan assay. Tissue lysates were incubated overnight with Proteinase K, DNase1 and RNase as previously described (Wolf et al. 2011), then GAG contents assessed using the Blyscan™ Sulfated Glycosaminoglycan Assay kit (biocolor life science assays, Accurate Chemical). Blyscan glycosaminoglycan standard 100 μg/mL (CAT. #CLRB 1010: Accurate Chemical, NY) was used to make a daily standard curve. Absorbance was measured at 656 nm using a Synergy MX plate reader and spectrophotometer (Bio Tek) with the Gen5 plate reader program. The blank value was subtracted from all readouts. Tissue GAG content is reported in ug GAG per mg protein, and urine GAG content is reported as ug GAG per mg creatinine. Urine creatinine was measured using the Creatinine Assay Kit (Sigma-Aldrich®) according to the manufacturer's instructions

Quantitative real-time PCR (qPCR) for IDS vector sequences. Tissue homogenates were mixed with 300 μlLcell lysis buffer (5 Prime) and with 100 μg of proteinase K, gently rocking overnight at 55° C. DNA was isolated from the sample by phenol/chloroform extraction. Reaction mixtures of 20 μl contained 60 ng of DNA template, 10 μl of FastStart Taqman Probe Master mix (Roche), 200 nM each of forward and reverse primers and 100 nM of Probe. A C1000 Touch™ Thermo Cycler (BIO-RAD) equipped with CFX manager software version 3.1 was used for qPCR reaction. The PCR conditions were: 95° C. for 10 minutes, followed by 40 cycles of 95° C. for 15 seconds and 60° C. for 1 minute. IDS primers used were forward primer: 5′-GCCAAAAATTATGGGGACAT-3′ (SEQ ID NO:1); IDS reverse primer: 5′-ATTCCAACACACTATTGCAATG-3 (SEQ ID NO:2)′; IDS probe: 6FAM-ATGAAGCCCCTT GAGCATCTGACTTCT-TAMRA (SEQ ID NO:3) To prepare the standard, pENN.AAV.CB7.hIDS was linearized by digestion with Sall restriction enzyme (New England BioLab Inc.). The linearized plasmid DNA was then purified using the 5Prime DNA Extraction kit. The plasmid DNA concentration was measured using a NanoDrop 1000 spectrophotometer (Thermo Scientific) with NanoDrop 1000 3.7.0 program. The purified linearized plasmid DNA was then diluted to prepare the qPCR standard curve. UltraPure™ distilled water (Invitrogen) was used as negative control. A 10-fold dilution series of linearized plasmid was used to generate a standard curve with a range of 1 to 10⁸ plasmid copies per assay in duplicate with amplification efficiencies between 90%-110% and R² of 0.96-0.98. Vector copy was calculated based on a daily standard curve and expressed as vector genomes per cellular genome equivalent (vg/ge).

Neurocoqnitive testing in the Barnes maze. The Barnes maze (Barnes 1979) is a circular platform measuring approximately 4 feet in diameter and is elevated approximately 4 feet from the floor with 40 holes spaced equally around the perimeter. All of the holes are blocked except for only one hole that is open for the mouse to escape the platform. Different visual cues were attached to each of the 4 walls for the mouse to use as spatial navigators. At 6 months of age, test mice were placed in the middle of the platform with an opaque funnel covering the mouse. The cover was lifted, releasing the mouse and exposing it to bright light. The animal is expected to complete the task by escaping the platform using the one open hole within 3 minutes. Each mouse was subjected to 4 trials per day for a total of 6 days. The time that the mouse required to escape the platform in each trial was recorded and the average was calculated for each day in each group.

Statistical analysis. Data are reported as mean±S.E. Statistical analyses were performed using Prism 6. Two-way ANOVA with Tukey's post-test was used to evaluate the significance of differences among test groups for IDS assay, GAG assay, and neurobehavioral assay with P value less than 0.05 considered significant. A two-tail t-test on Microsoft Excel was used to evaluate differences in IDS activities between the left and the right hemispheres of microdissected brain.

### Results

Comparison of vector constructs and route of administration to achieve IDS expression in the CNS. A pilot study was conducted to compare several AAV vector constructs and to find a suitable route of administration resulting in IDS expression in the CNS. NOD.SCID mice were used for this study to circumvent the potential complication of an anti-IDUA immune response. The 4 vectors (AAV9.hIDS, AAV9.hIDSco, AAV9.hIDS-hSUMF1, and AAV9.hIDSco-hSUMF1co) were delivered by intrathecal (IT) administration. SUMF1 encodes an enzyme which post-translationally modifies an amino acid in sulfatases, including IDS, resulting in conversion into catalytically active forms. The addition of SUMF1 to some of the vectors was to determine if SUMF1 activity is rate-limiting in producing active IDS protein when IDS is overexpressed. Five untreated IDS+NOD.SCID mice were used as a control group. Six weeks post injection, the mice were euthanized, harvesting and microdissecting the brain into different portions. Parallel studies in MPS I have demonstrated supraphysiological activities of IDUA in the CNS post-IT injection of AAV9 vector encoding hIDUA (Belur et al. 2014). It was expected to see high levels of IDS, exceeding the endogenous level in IDS+NOD.SCID mice administered AAV9.hIDS vector. Surprisingly, no significant increase of IDS activity was observed in the CNS exceeding the endogenous level observed in uninjected NOD.SCID mice regardless of vector construct (data not shown). AAV9.hIDS was also injected into 2 groups of 3 wild type C57BL/6 (IDS+C57BL/6) mice at 8 weeks of age, one group via IT administration and the other group via intravenous administration (IV). Again no significant increase in the level of IDS activity was observed in the CNS above the endogenous level of untreated controls (data not shown). Thus, neither IT nor IV injection of IDS-encoding AAV vector appeared to be a suitable route of administration.

Another unexpected result from the initial study described above is that while there was undetectable increase in IDS activity in the CNS, plasma IDS activity in both IV and IT treated groups was increased up to approximately 140-fold above the untreated wild type level and persisted for at least 12 weeks post-treatment. For the IT treated animals this result suggests that AAV vector was distributed to the peripheral circulation after injection into the cerebrospinal fluid. The presence of sustained enzyme activity for at least 12 weeks post-injection (either IT or IV) also suggests that hIDS is presumably nonimmunogenic for C57BL/6 mice.

The same 4 vector constructs (AAV9.hIDS, AAV9.hIDSco, AAV9.hIDS-hSUMF1, and AAV9.hIDSco-hSUMF1co) were administered to immunocompetent MPS II mice by intracerebroventricular injection, a procedure that supports a much higher level of transduction in the CNS than IT injection. Immunosuppression of the MPS II test animals was not necessary since it was found that expression of human IDS does not elicit an immune response in C57BL/6 mice. An additional group of MPS II mice was injected ICV with a combination of 2 vectors: AAV9.hIDS and AAV9-hSUMF1 (at a 1:1 ratio (AAV9.hIDS+AAV9.hSUMF1; at a dose of 5×10¹⁰ vg total) to determine if there would be additional IDS activity under conditions in which SUMF1 expression is optimized as compared to driving expression from an IRES. Untreated wild type littermates were used as controls. Six weeks post injection the animals were euthanized, organs were harvested, and brains were microdissected to determine IDS activity. Animals injected with AAV9.hIDS, AAV9.hIDS-hSUMF1, or AAV9.hIDS+AAV9.hSUMF1 showed levels of IDS activity approximately 10% to 40% of the wild type level in most portions of the brain IDS activity was undetectable in all areas of the brain in MPS II mice. Animals injected with codon-optimized vector constructs showed mostly less than 10% of the wild type level. There was no significant difference between AAV9.hIDS injected animals and animals injected with AAV9.hIDS plus hSUMF1. Thus in our hands co-delivery of hSUMF1 either on the same vector or on a separate vector did not enhance the level of IDS activity assessed. Vector AAV9.hIDS was subsequently used for more extensive efficacy studies in ICV administered MPS II mice as described below, as neither the addition of SUMF1 nor the codon-optimization algorithm used resulted in increased IDS activity as compared to the native hIDS cDNA sequence.

Prevention of CNS and peripheral lysosomal disease by Intracerebroventricular administration of AAV9.hIDS vector. A dose of 5.6×10¹⁰ AAV9.hIDS vg was infused into eight-week old MPS II mice by ICV injection to achieve widespread CNS distribution of the vector through the cerebrospinal fluid (CSF). As in the pilot study, plasma IDS activities were observed up to 160-fold higher than wild-type in this larger cohort of ICV-treated MPS II animals, and this expression persisted throughout the experiment (28 weeks post injection). Urine was collected at the end of the study (week 40 post-injection) to evaluate the effect of long-term IDS expression on GAG excretion in the treated animals compared to wild type and untreated MPS II mice. Urine GAG was significantly elevated in MPS II animals when compared to wild-type littermates (p<0.05). The treated animals demonstrated a significant reduction in urine GAG content (p<0.05) when compared to untreated littermates and were normalized when compared to the wild type level (p>0.05).

At 10 months of age (40 weeks post injection) all mice were euthanized and organs were harvested for analysis. IDS activity was undetectable in all areas of the brain and spinal cord of untreated MPS II mice. AAV9.hIDS injected animals had IDS activity in all regions of the brain at approximately 9% to 28% of wild type, 53% in olfactory bulb and 7% in the spinal cord. Although the vector was infused into the right ventricle of the brain, we did not observe a significant difference in IDS activity between the left and the right hemispheres (p>0.05). Unlike the CNS, supraphysiological levels of enzyme activity were observed in all tested peripheral organs such as heart, liver, spleen and kidney (11-, 166-, 5-, and 3-fold, respectively), except in the lung where it was observed to be 34% of wild type.

DNA was isolated from the same tissue homogenates and evaluated for vector distribution by qPCR. Consistent with Wolf et al. (Wolf et al. 2011), ICV infusion of AAV vector resulted in global distribution of vector in the CNS and in all of the tested peripheral tissues. We observed the highest vector copy number in the right hippocampus (49 vg/ge), while similar vector copies were observed between the left and the right hemisphere for all regions of the brain. Unlike the CNS, relatively low copy numbers were detected in most tested peripheral tissues include heart, lung, spleen and kidney of the treated animals (less than 0.6 vg/ge), while high vector copy number was observed in the liver (44 vg/ge). This suggested that enzyme produced by the liver was released into the circulation where the tested peripheral organs took up the circulating enzyme (i.e. metabolic cross-correction).

The global distribution and expression of IDS had a significant effect on accumulation of lysosomal storage materials. Elevation of lysosomal GAG content was observed in the CNS of untreated MPS II mice when compared to wild type littermates (; p<0.0001). Even though there was only 10% to 40% of wild type IDS level in the CNS, GAG content in the CNS was normalized when compared to wild type (p<0.01). Similar to the CNS, significant elevation of lysosomal GAG content was observed in all tested peripheral organs of the untreated MPS II animals when compared to wild type (p<0.01). In contrast, significantly decreased GAG levels were observed in all tested peripheral tissues of the treated mice when compared to the untreated group (p<0.01). Statistical analysis showed no significant difference in GAG content between wild type animals and the treated group (p>0.05), which indicates that GAG content was normalized in the treated group.

The body weights of all mice were measured before sacrificed, and organs were weighed after the animals were perfused with 1×PBS, calculating the percentage of organ weight to body weight immediately post-sacrifice. We observed no significant difference in the size of heart, lung, spleen, and kidney amongst all groups. However, the liver of untreated MPS II mice was 20% larger than that of wild type animals (6.2% and 5.2% of total body weight, respectively; p<0.001). In contrast, the liver of the treated MPS II mice was 68% smaller than the untreated group (4.2% and 6.2% of total body weight, respectively; p<0.0001). This result shows that normalization of GAG content in the liver in turn prevented hepatomegaly in the treated mice.

Sustained expression of IDS in the CNS leads to prevention of neurocognitive deficit in MPS II mice. At 6 months of age (4 months post-treatment), untreated MPS II mice, AAV9.hIDS treated MPS II mice and control normal littermates were evaluated for neurocognitive function in the Barnes maze, a test for spatial navigation and memory. The animals were subjected to a series of 3-minute trials, 4 trials per day for a course of 6 days. Wild type littermates showed reduced latency to escape (30 s) on day 6, while untreated MPS II mice exhibited a significant deficit in learning this task (latency to escape reduced only to 71 seconds; p≤0.05 vs normal littermates). In contrast, the AAV9.hIDS treated mice showed a marked reduction in latency to escape (25 s), significantly outperforming the untreated MPS II mice (p≤0.01) on day 5 and day 6. In addition, there was no significant difference between the treated animals and wild type littermates (p>0.05). We conclude that sustained expression of IDS in the CNS prevents the emergence of neurocognitive deficits in MPS II mice when treated at a young age.

### Discussion

In this study, AAV9.hIDS was administered using a strong promoter to the CNS of MPS II mice. Levels of IDS activity in the CNS were only 7% to 28% of wild type. In contrast, levels of IDS activity in the circulation and in tested peripheral organs were at least 2-fold and up to 170-fold higher than wild type levels. It was also observed that sustained IDS expression leads to global normalization of GAG content. Finally, it was observed that sustained levels of IDS activity had a profound effect on preventing neurologic deterioration.

No significant increase in IDS activity was observed in the CNS above the endogenous level in IV or IT treated IDS+ mice regardless of vector construct, route of administration, or mouse strain, even though IDS was expressed under regulation of the strong CB7 promoter. Similar results were also observed when AAV9.hIDS was infused into MPS II mice via ICV injection; although it was observed IDS activity in the CNS of these mice above baseline, it was lower than wild type levels at 40 weeks post-treatment. Similar results were reported from Motas et al. (Motas et al. 2016) and Hinderer et al (Hinderer et al. 2016), in which they observed approximately 20% to 40% of wild type levels in the CNS. These limited levels of IDS activity are in stark contrast with the levels of IDUA activity observed in the CNS of MPS I mice after ICV injection of AAV9-IDUA vector, in which 100- to 1000-fold higher than wild type levels of IDUA activity are observed (Belur et al. 2014). Supraphysiological levels of IDS (>1000 nmol/hr/mg) were also observed in the liver of ICV administered animals in our study at a similar vector copy number that yielded 10- to 100-fold less IDS expression (10 to 100 nmol/hr/mg) in the CNS. Highly relevant to the goal of CNS-directed gene therapy for MPS II, therefore is this question: What is it that limits expression of IDS in the brain after highly efficient AAV mediated IDS gene delivery?

One possibility is that SUMF1 activity might be rate limiting for the generation of active IDS in the brain. SUMF1 is required for the post-translational activation of lysosomal sulfatases, including IDS (Sabourdy et al. 2015). hIDS enzyme clearly was activated in tissues of the MPS Il mouse, presumably by mouse SUMF1, but this process could be rate limiting in the brain, if AAV-encoded hIDS protein is expressed in a large quantity but only a limited amount of hIDS becomes activated. For example, Fraldi et al (Fraldi et al. 2007) demonstrated that N-sulfoglucosamine sulfohydrolase activities were increased when the enzyme was co-expressed with SUMF1 in their MPS IIIA studies. Anticipating a potential limitation of SUMF1, we co-transduced hIDS and hSUMF1 either on the same construct or on 2 separate vectors, but we found no significant increase in hIDS activity compared to delivery of hIDS alone. We concluded that hIDS activity was not enhanced by co-delivery with hSUMF1 in these experiments. Further studies evaluating SUMF1 mediated activity of IDS in the CNS are nonetheless warranted.

Another possibility is that the CB7 promoter might be limiting when compared to the endogenous IDS promoter. To further investigate this possibility, IDS activity (nmol/h/mg protein=unit) per vector copy was calculated for each tissue and compared to IDS activity per endogenous copy in male mice. IDS activities in the CNS of the treated mice were observed between 2 and 32 units per vector copy compared to wild type male mice, which express an average of 200 units per genome equivalent (approximately only 1% to 31% of wild type level). From this one might conclude that the CB7 promoter is not as robust as the endogenous IDS promoter in the brain. However, the lower level of vector mediated IDS expression per vector copy vs endogenous expression most likely results from the presence of excess AAV vector in the brain post-injection that does not become intracellularized and expressed. Nonetheless, the MPS Il results contrast greatly with the results from the present MPS I studies in which levels of IDUA activity at approximately 10- to 100-fold higher than wild type were observed in the CNS of mice administered AAV-hIDUA intrathecally. (Belur et al. 2014), while heterozygous MPS I animals express approximately 6 units per genome equivalent in the brain (Ou et al. 2014). Therefore it is feasible to achieve or exceed wild type IDUA levels in the CNS of MPS I mice. The promoter that was used in the MPS Il study was similar but not identical to the promoter used in the MPS I studies (Wolf et al. 2011; Belur et al. 2014), and so relative promoter strength may have contributed to the observed differences in outcome.

Surprisingly, supranormal levels of enzyme activity were observed in all tested peripheral organs of the treated animals. The highest levels of IDS activity (164-fold wild type) were observed in the liver, which correlated with the highest number of vector copies found (48 vc/ge). In contrast, the tested peripheral organs (other than liver) contained low levels of vector, but higher than wild type levels of IDS activity. The exceptionally high levels of IDS in the liver lead to sustained high levels of IDS activity (up to 172-fold higher than wild type) in the circulation. Similar results have been reported in a MPS IIIA study and several MPS I studies using different types of vectors (Aronovich et al. 2009; Osborn et al. 2011; Haurigot et al. 2013), along with 2 studies in MPS II mice (Motas; Hinderer). These results suggest that in our study liver acts as an enzyme factory that produces an enormous amount of IDS and releases it into the circulatory system where it is subsequently taken up by the other organs in the periphery. Even though we observed higher than wild type levels of IDS enzyme activity in heart, spleen and kidney in the treated mice, the vector levels in these organs were unexpectedly low (less than 0.6 vc/ge). This low transduction rate suggests that these organs took up circulating IDS leading to metabolic cross-correction with higher levels of IDS than wild type.

Polito et al. showed no IDS activity in the CNS after a single IV injection of AAV2/5 CMVhIDS. However, they observed partial correction of GAG content in the CNS (Polito and Cosma 2009). They speculated that only a fraction of high level circulating hIDS penetrated the BBB into the CNS with subsequent partial correction of GAGs. Similarly, even though there were exceptionally high levels of IDS in the circulation of AAV9.hIDS treated mice, we observed only subphysiological activities of the enzyme in the CNS. This finding indicates that only insignificant amounts of the circulating enzyme if any were able to penetrate the BBB into the CNS. Therefore the level of IDS observed in the brain most likely relied on expression of the vector construct inside the CNS rather than from the circulating enzyme. Further investigation is needed to achieve IDS levels comparable to or higher the wild type in the CNS.

Sustained levels of enzyme expression after direct injection into the CNS have been shown to have profound effects on GAG reduction in several MPS studies whether or not wild type levels of those enzymes were achieved (Belur et al. 2014; Wolf et al. 2011; Motas et al. 2016; Haurigot et al. 2013) (Hinderer et al 2016). Similarly, although we observed only subphysiological levels of IDS in the CNS after ICV injection of AAV9.hIDS, there was a significant effect of the enzyme on GAG reduction that was greater than expected. Desnick et al. and Polito et al. speculated that only less than 5% of the wild type level of lysosomal enzyme is needed to correct a GAG storage defect (Desnick and Schuchman 2012; Polito and Cosma 2009). The present results are consistent with this speculation. After AAV9.hIDS treatment, it was found that all areas of the brain and even spinal cord (the organ with the lowest enzyme measured; 10.7 nmol/h/mg protein; 7.4% of wild type) showed significantly lower GAG content than untreated MPS II mice. Statistical analysis revealed striking results as GAG content in the CNS of the treated mice was normalized when compared to wild type. We also observed normalization of GAG content in the urine and in heart, lung, liver, spleen and kidney of the treated animals. These results indicate a sustained effect of IDS expression on correction of GAG content.

Roberts et al. demonstrated a direct relationship between GAG accumulation and liver size when injecting rodamine B, a GAG synthesis inhibitor, into MPS IIIA mice. They found that GAG content was decreased in the liver, leading to normalization of liver size (Roberts et al. 2006). Motas et al. observed a preventive effect on hepatomegaly after ICV administration of AAV9-hIDS into MPS II mice (Motas et al. 2016). Similarly, it was also observed that the weight of the liver in treated mice was normalized after ICV injection of AAV9.hIDS. This result indicates a profound effect of sustained IDS expression leading to normalization of GAG content in the liver, which in turn prevents hepatomegaly in the treated mice.

Several MPS studies have demonstrated prevention of neurologic deficits after AAV-mediated gene transfer in mice (Fu et al. 2011; Wolf et al. 2011; Motas et al. 2016). It was observed neurocognitive deficiency in untreated MPS II mice at 6 months of age. We also observed that sustained IDS expression in the CNS in prevented the emergence of neurocognitive dysfunction in MPS II mice after ICV infusion of AAV9.hIDS. Several studies have shown that the hippocampus is associated with neurocognition in rodents (Seeger et al. 2004; Paylor et al. 2001; Miyakawa et al. 2001). However, we cannot exclude the possibility that physical impairment such as vision, olfactory sense, or motor neuron defects could also affect our results in the Barnes maze, as rodents require all of the aforementioned physical capabilities in order to perform the required task in this test. (Harrison et al. 2006). Additional behavioral analyses would support our observation that neurocognitive deficit plays a pivotal role in learning impairment of untreated MPS II mice and its prevention by AAV mediated IDS gene transfer.

In conclusion, the present results show the benefit of direct AAV9-mediated hIDS gene transfer to the CNS. However, the limited level of AAV-mediated IDS expression achieved in the CNS in comparison with other tissues such as liver, and in comparison with the expression of other therapeutic genes introduced into the CNS by AAV mediated transduction, such as IDUA (Belur et al. 2014; Wolf et al. 2011), was surprising. Nonetheless, the MPS II data indicate that direct injection of AAV9-hIDS vector into the CNS resulted in efficient gene transfer that is key to treatment of MPS II and prevention neurocognitive deficits. We found that the AAV9.hIDS vector was capable of not only crossing the BBB resulting in global transduction of the vector both inside and outside of the CNS, but also providing long-term expression of IDS enzyme systemically. Sustained IDS expression corrected the accumulation of GAG in liver and subsequently prevented the emergence of hepatomegaly. In addition, our results reinforce the importance of sustained IDS expression in the CNS in preventing the emergence of neurologic deficits when animals are treated at a young age.

### Example IIIA

### Materials and Methods

### AAV Vector Assembly and Packaging

All vectors were constructed, packaged, and purified at the Penn Vector Core (Philadelphia, PA) and were provided by REGENXBIO, Inc. (Rockville, MD). In brief, the expression cassettes contained a chicken beta-actin (CB7) promoter with cytomegalovirus (CMV) enhancer followed by hIDS or human sulfatase modifying factor 1 (hSUMF1), rabbit beta-actin polyadenylation signal on the backbone of AAV2 inverted terminal repeats (ITR) on both 3′- and 5′-ends. Co-expression constructs included an internal ribosome entry site (IRES) positioned between hIDS and SUMF1 to initiate translation of SUMF1 downstream of the IRES. In this study we investigated five different vector constructs: AAV9 expressing human IDS alone (AAV9.hIDS); AAV9 expressing codon optimized human IDS (AAV9.hIDSco); AAV9 co-expressing human IDS and human SUMF1 (AAV9.hIDS-hSUMF1); AAV9 co-expressing codon-optimized human IDS and codon-optimized human SUMF1 (AAV9.hIDScohSUMF1co); and AAV9 expressing human SUMF1 alone (AAV9.hSUMF1). AAV vectors were packaged by co-transfecting three plasmids—AAV cis, AAV trans (pAAV2/9 rep and cap), and adenovirus helper (pAdDF6)—into HEK 293 cells (Lock et al., 2010). AAV vector was then purified from supernatants using a Profile II depth filter and concentrated by tangential flow filtration (TFF). The concentrated feed stock was reclarified by iodixanol gradient centrifugation and then re-concentrated using a TFF cassette with a 100 kDa MWCO HyStream screen channel membrane. The purified vector was then tested for purity by SDS-PAGE and for potency by quantitative polymerase chain reaction (qPCR) (Lock et al., 2010).

### Animal Care and Husbandry

All animal care and experimental procedures were conducted under approval of the Institutional Animal Care and use Committee (IACUC) of the University of Minnesota. NOD.SCID mice were purchased from The Jackson Laboratory and C57BL/6 wild-type mice were purchased from National Cancer Institute. C57BL/6 iduronate-2-sulphatase knockout (IDS KO) mice were kindly provided by Dr. Joseph Muenzer (University of North Carolina, NC) and maintained under specific pathogen-free conditions at the Research Animal Resources (RAR) facilities of the University of Minnesota. MPS II male pups (IDS^{-/0}) were generated by breeding heterozygous (IDS^{+/-}) females to wild type (IDS^{+/0}) C57BL/6 males. All pups were genotyped by PCR.

### AAV Vector Administration

For intrathecal (IT) injections, eight-week-old mice were injected with a dose of 5.6×10¹⁰ vector copies (vc) of AAV9 vector between the L5 and L6 vertebrae, as previously described (Vulchanova et al., 2010). The injection was performed in conscious animals in a 10-15 second duration. For intravenous (IV) injections, animals were briefly restrained and injected via the tail-vein with a dose of 5.6×10¹⁰ vc. Intracerebroventricular (ICV) injections were carried out in adult 8-week-old mice, as previously described (Janson et al., 2014). Briefly, animals were injected intraperitoneally with a ketamine/xylazine mixture (100 mg/kg ketamine, 10 mg/kg xylazine) to produce deep anesthesia and then mounted in a stereotactic frame (Kopf Model 900). An incision was made to expose the cranium, a small hole was drilled as a site for the injection, and then a Hamilton syringe (Model 701) was used to carry out the infusion at a rate approximately 0.5 IL/minute by hand. The syringe was left in place for an additional 3 min and then slowly withdrawn over a period of at least 2 minutes. The scalp was sutured after completion of the injection, and after recovery from the anesthesia, the mouse was returned to standard housing. All of the mice received a 3-day course of Ketoprofen (2.5-5.0 mg/kg) subcutaneously and Baytril 5 mg/kg intraperitoneally to prevent infection and inflammation post surgery.

### Sample Collection and Preparation

Blood was collected by submandibular puncture using sterile 5 mm lancets (Goldenrode™) into Microvette® heparinized coated tubes (Sarstedt AG & Co.) and centrifuged in an Eppendorf centrifuge 5415D at 7,000 rpm for 10 min. Plasma was collected and stored at −20° C. to −80° C. for IDS assay. Urine was collected and stored at −20° C. until used for creatinine and GAG assay. Organs were harvested by first determining animal weight using an OHAUS© CS 200 scale before euthanasia using a CO₂ fume chamber at 2 L/minutes for 3 minutes. The animals were perfused with 60 mL of 1× phosphate-buffered saline (PBS) in a 60 mL syringe (BD) with a SURFLO® winged infusion set (TERUMO®) size 23G·×¾″ by hand pressure. The heart, lung, liver, spleen, kidney, and spinal cord were harvested and weighed using a Sartorius BP 61S scale. The brain was micro-dissected into left and right cerebellum, cortex, hippocampus, striatum, olfactory bulb, and thalamus/brainstem. The organs were immediately snap frozen and stored at −70° C. until further tissue processing.

For tissue processing, the cerebellum, hippocampus, striatum, and olfactory bulb were added into preassigned 1.5 mL locked-cap microtubes (Eppendorf) containing one scoop (0.2 g/scoop) of 0.5 mm glass beads (Next Advance) in 250 μL of sterile saline solution. The thalamus/brainstem, cortex, and spinal cord were added into assigned locked-cap microtubes containing two scoops of 0.5 mm glass beads in 400 IL of sterile saline solution. Half of the lung and the whole spleen were added into assigned tubes containing two scoops of 0.9-2.0 mm stainless steel blend (0.6 g/scoop) in 400 μL of saline solution. The heart, about 0.3 g of liver, and one kidney were added into assigned tubes containing three scoops (a mixture of two scoops of 0.9-2.0 mm stainless steel blend [0.6 g/scoop] and one scoop of 3.2 mm stainless steel beads [0.7 g/scoop]) in 600 μL of sterile saline solution. All of the prepared samples in the bead tubes were then homogenized using a Bullet Blender® STORM bead mill homogenizer (Next Advance) at speed 12 for 5 minutes to generate tissue homogenate. Fifty microliters of tissue homogenates were transferred into 1.5 mL microtubes (GeneMate) and stored at −20° C. to −80° C. for qPCR. The remaining tissue homogenates were clarified using an Eppendorf centrifuge 5424R at 13,000 rpm for 15 minutes at 4° C. All of the supernatants (tissue lysates) were transferred into new microtubes and stored at −20° C. to −80° C. until used for IDS, GAG, and protein assays.

### Iduronate Sulfatase Assay

IDS enzyme activity was measured in tissue lysates using 4-methylumbelliferyl-α-L-iduronide-2-sulphate disodium (4-MU-aldoA-2S; Toronto Research Chemical Incorporation; cat. #M334715) as substrate in a two-step assay. Tissue lysates were mixed with 1.25 mM MU-aldoA-2S (in 0.1 M sodium acetate buffer pH 5.0+10 mM lead acetate+0.02% sodium azide) and incubated at 37° C. for 1.5 hours. The first-step reaction was terminated with PiCi buffer to stop IDS enzyme activity (0.2 M Na₂HPO₄/0.1 M citric-acid buffer, pH 4.5+0.02% Na-azide). A final concentration of 1 μg/mL Iduronidase (IDUA; R&D Systems; cat. #4119-GH-010) was added into the tubes to start the second-step reaction. The tubes were incubated overnight at 37° C. to cleave 4-MU-IdoA into 4-MU. The second step reaction was terminated by adding 200 μL of stop buffer (0.5M Na₂CO₃+0.5 M NaHCO₃, 0.025% Triton X-100, pH 10.7). The tubes were centrifuged using an Eppendorf centrifuge 5415D at 13,000 rpm for 1 minute. Supernatants were transferred into a round-bottom black 96-well plate and fluorescence measured at excitation 365 nm and emission 450 nm, 75 sensitivity using a Synergy MX plate reader and spectrophotometer (Bio Tek) with Gen5 plate reader program. Enzyme activity is expressed in nmol/h/mL plasma for plasma samples and in nmol/h/mg protein for tissue extracts. Protein was determined using the Pierce™ 660 nm Protein Assay Reagent with bovine serum albumin as a standard (cat. #23208; Thermo Scientific).

### Glycosaminoglycan Assay

Tissue lysates were incubated overnight with Proteinase K, DNase1, and RNase, as previously described (Wolf et al., 2011), then, GAG contents were assessed using the Blyscan™ Sulfated Glycosaminoglycan Assay kit (Biocolor Life Science Assays; Accurate Chemical). Blyscan glycosaminoglycan standard 100 μg/mL (cat. #CLRB 1010; Accurate Chemical) was used to make a daily standard curve. Absorbance was measured at 656 nm using a Synergy MX plate reader and spectrophotometer (Bio Tek) with the Gen5 plate reader program. The blank value was subtracted from all readouts. Tissue GAG content is reported in micrograms GAG per milligrams protein, and urine GAG content is reported as micrograms GAG per milligrams creatinine. Urine creatinine was measured using the Creatinine Assay Kit (Sigma-Aldrich©) according to the manufacturer's instructions.

qPCR for IDS Vector Sequences

Tissue homogenates were mixed with 300 μL cell lysis buffer (5 Prime) and with 100 μg proteinase K, gently rocking overnight at 55° C. DNA was isolated from the sample by phenol/chloroform extraction. Reaction mixtures of 20 μl contained 60 ng of DNA template, 10 μL of FastStart Taqman Probe Master mix (Roche), 200 nM each of forward and reverse primers, and 100 nM of Probe36 (#04687949001; Roche). A C1000 Touch™ Thermo Cycler (Bio-Rad) equipped with CFX manager software v3.1 was used for qPCR reaction. The PCR conditions were: 95° C. for 10 minutes, followed by 40 cycles of 95° C. for 15 seconds and 60° C. for 1 minute. IDS primers used were: forward primer-5′-TCCCTTACCTCGACCCTTTT-3′ (SEQ ID NO: 4); IDS reverse primer-5′-CACAAGGTCCATGGATTGC-3′ (SEQ ID NO: 5). To prepare the standard, pENN.AAV.CB7.hIDS was linearized by digestion with Sall restriction enzyme (New England BioLab, Inc.). The linearized plasmid DNA was then purified using the 5Prime DNA Extraction kit. The plasmid DNA concentration was measured using a NanoDrop 1000 spectrophotometer (Thermo Scientific) with the NanoDrop 1000 3.7.0 program. The purified linearized plasmid DNA was then diluted to prepare the qPCR standard curve. UltraPure™ distilled water (Invitrogen) was used as negative control. A10-fold dilution series of linearized plasmid was used to generate a standard curve with a range of 1-10⁸ plasmid copies per assay in duplicate with amplification efficiencies between 90% and 110% and R² of 0.96-0.98. Vector copy was calculated based on a daily standard curve and expressed as vector copies per cellular genome equivalent (vc/ge).

### Neurocognitive Testing in the Barnes Maze

The Barnes maze (Barnes, 1979) is a circular platform measuring approximately 4 feet in diameter and is elevated approximately 4 feet from the floor with 40 holes spaced equally around the perimeter. All of the holes are blocked except for only one hole that is pen for the mouse to escape the platform. Different visual cues were attached to each of the four walls for the mouse to use as spatial navigators. At 6 months of age, test mice were placed in the middle of the platform with an opaque funnel covering the mouse. The cover was lifted, releasing the mouse and exposing it to bright light. The animal is expected to complete the task by escaping the platform using the one open hole within 3 minutes. Each mouse was subjected to four trials per day for a total of 6 days. The time that the mouse required to escape the platform in each trial was recorded, and the average was calculated for each day in each group.

### Statistical Analysis

Data are reported as mean—standard error (SE). Statistical analyses were performed using Prism 6. Two-way analysis of variance with Tukey's post test was used to evaluate the significance of differences among test groups for IDS assay, GAG assay, and neurobehavioral assay, with a p-value of <0.05 considered significant. A two-tailed t-test on Microsoft Excel was used to evaluate differences in IDS activities between the left and the right hemispheres of the microdissected brain.

### Results

### Pilot Studies: Comparison of Vector Constructs and Route of Administration to Achieve IDS Expression in the CNS

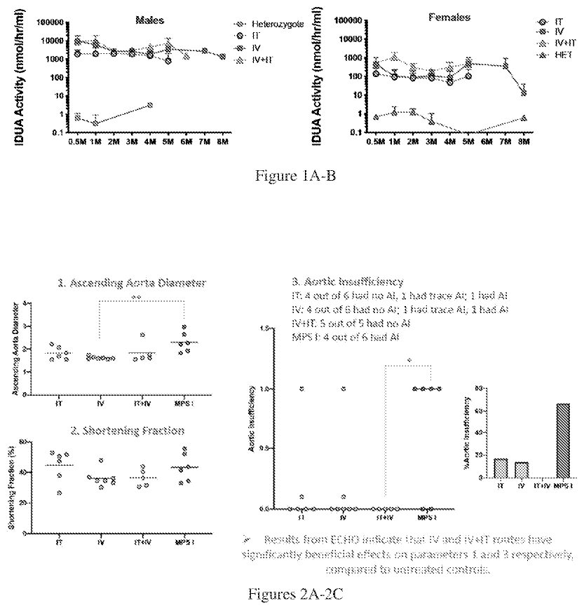

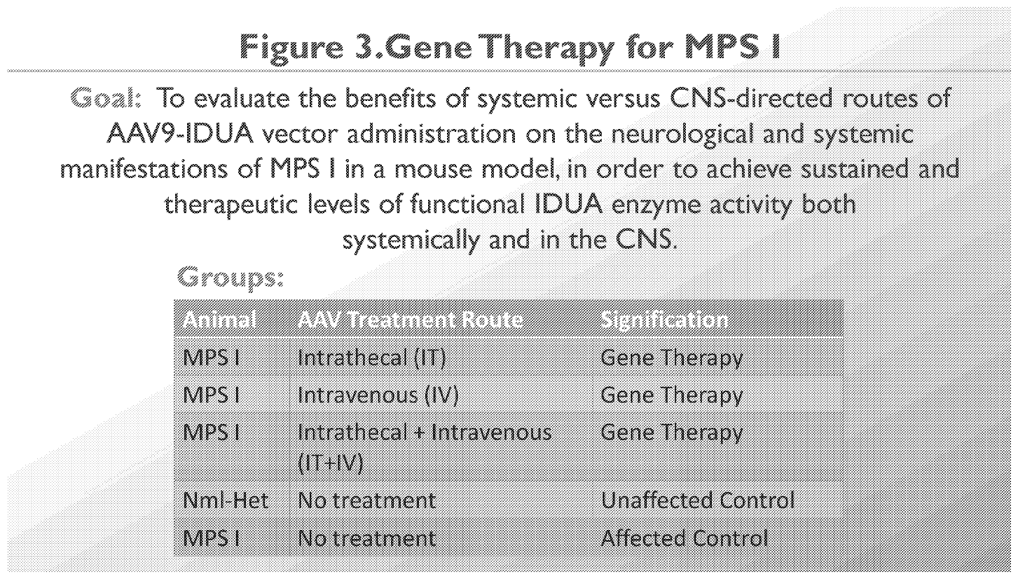

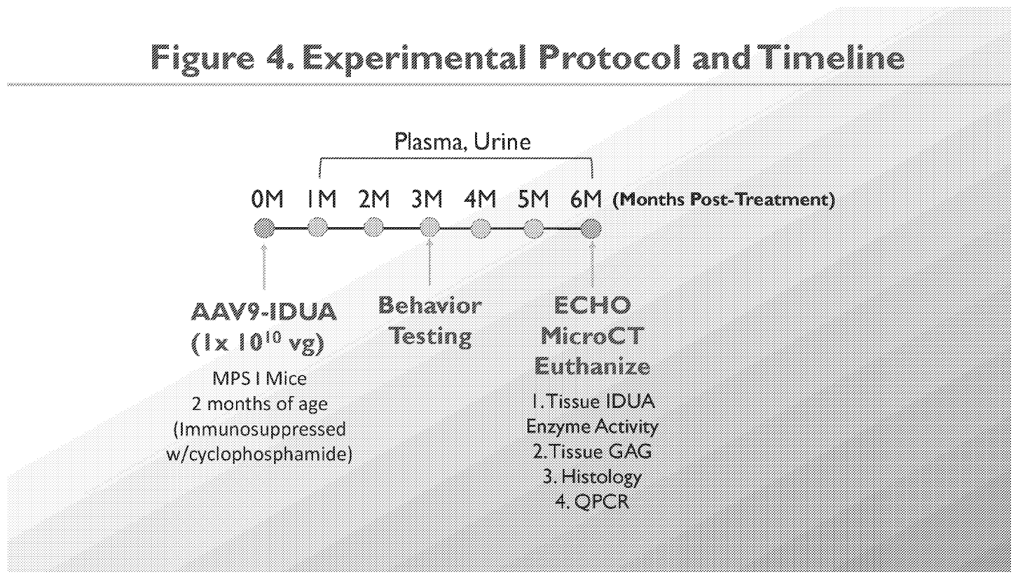

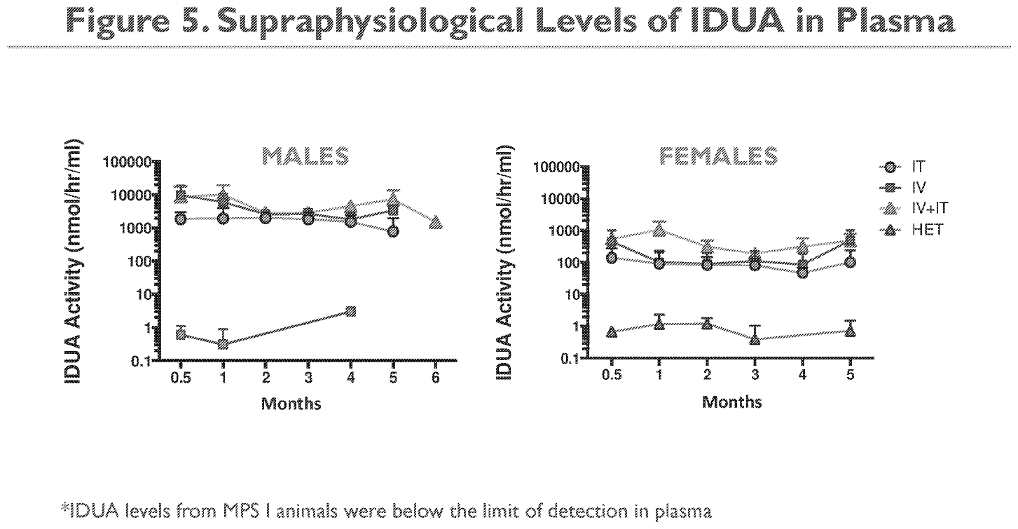

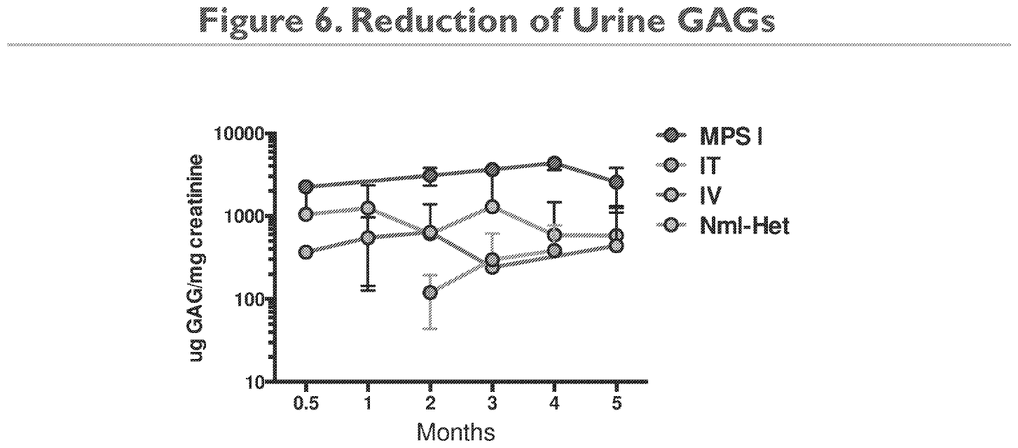

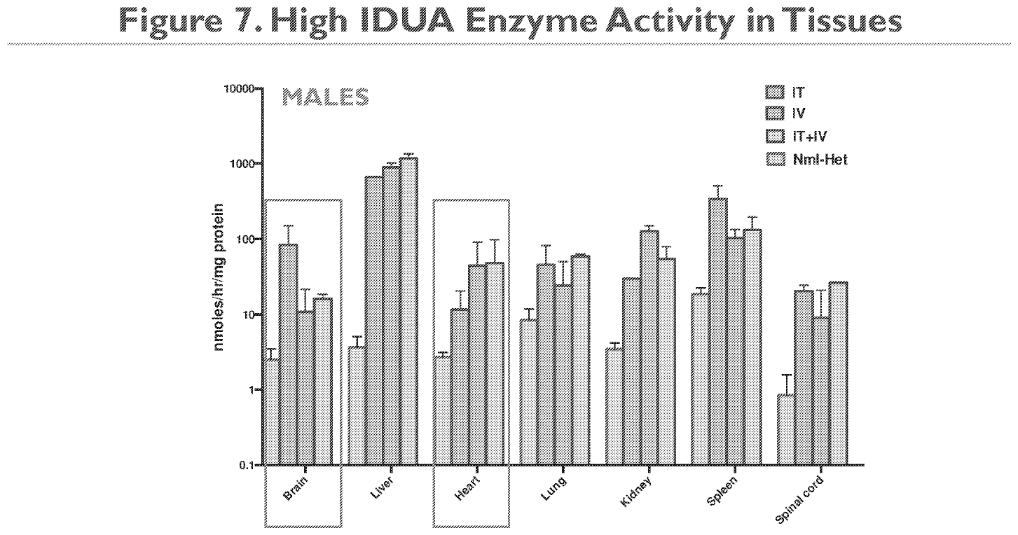

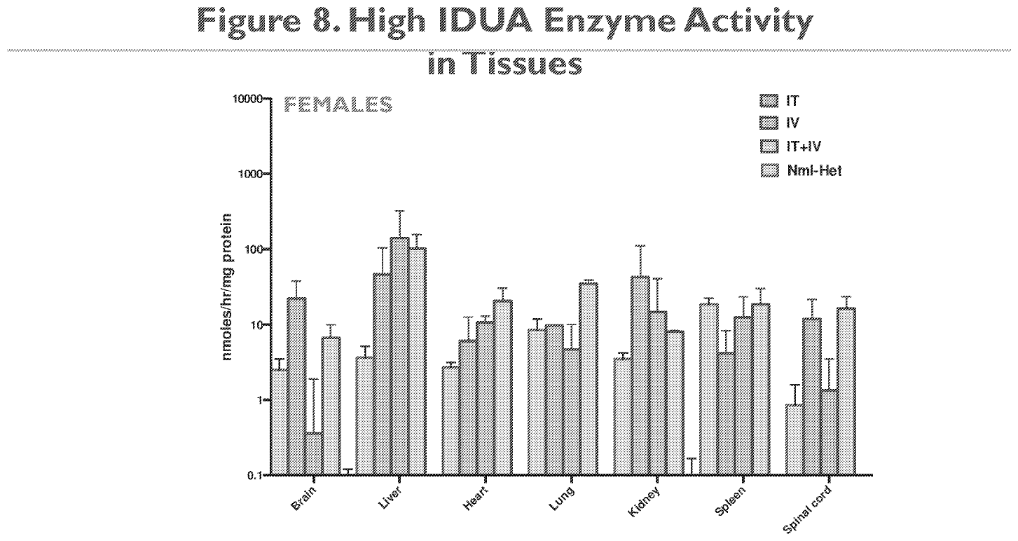

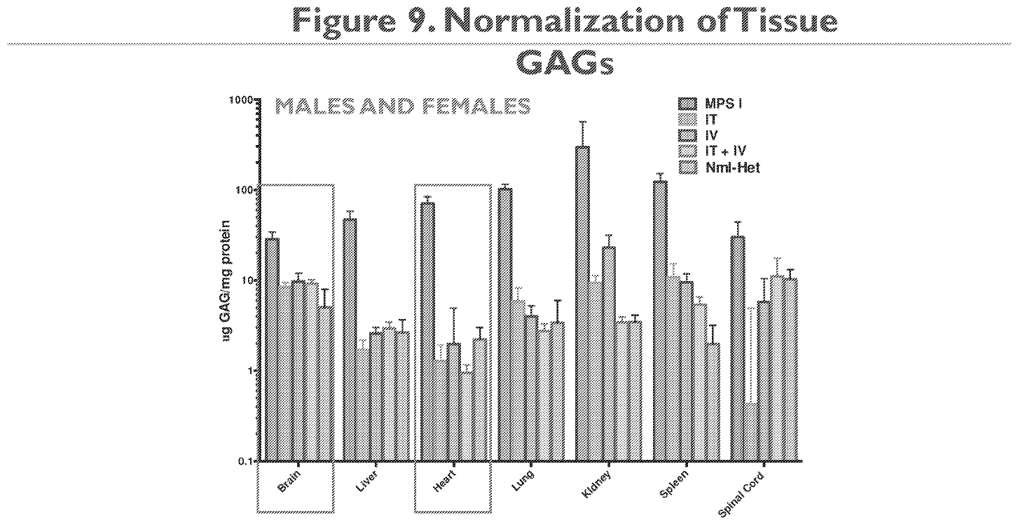

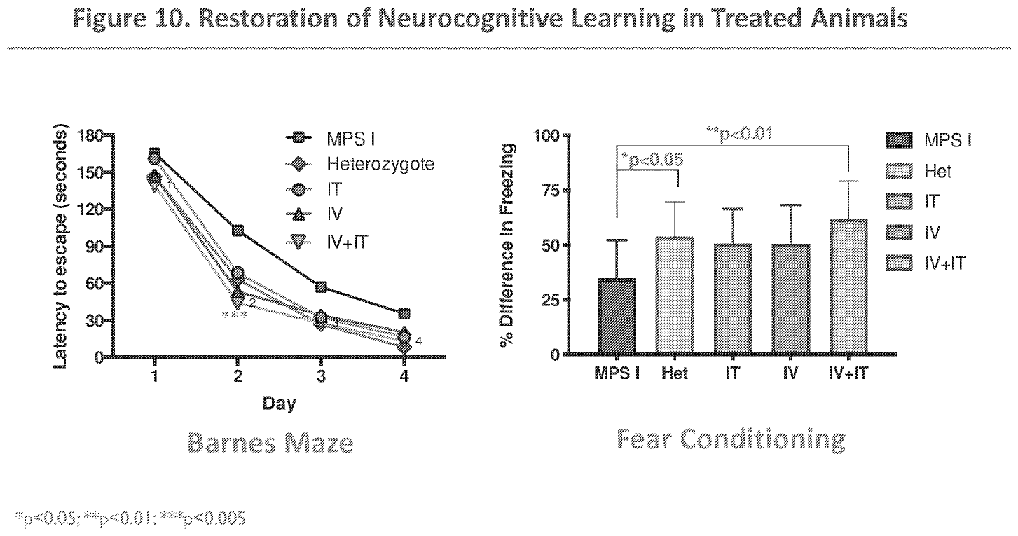

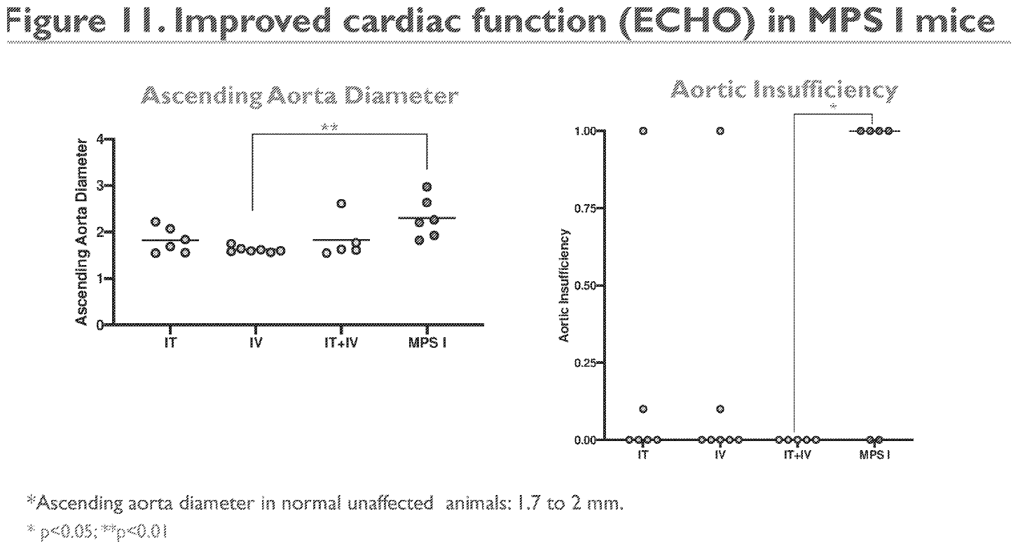

A pilot study was conducted to compare several AAV vector constructs and to find a suitable route of administration resulting in IDS expression in the CNS. NOD.SCID mice were used for this study to circumvent the potential complication of an anti-IDS immune response. The four vectors shown in FIG. 1A-D (AAV9.hIDS, AAV9.hIDSco, AAV9.hIDS-hSUMF1, and AAV9.hIDSco-hSUMF1co) were delivered by IT administration, as described in the Materials and Methods. SUMF1 encodes an enzyme which post-translationally oxidizes an active site cysteine in lysosomal sulfatases, including IDS, converting the enzyme into a catalytically active form (Sabourdy et al., 2015). The addition of SUMF1 to some of the vectors was to determine if SUMF1 activity is rate-limiting in producing active IDS protein when IDS is overexpressed. Five untreated IDS+NOD.SCID mice were used as a control group. Six weeks post injection, the mice were euthanized, and the brain was microdissected into different portions. Our parallel studies in MPS I have demonstrated supraphysiological activities of IDUA in the CNS post-IT injection of AAV9 vector encoding hIDUA (Belur et al., 2014). We thus expected to see high levels of IDS, exceeding the endogenous level in IDS+NOD.SCID mice administered AAV9.hIDS vector. Surprisingly, we observed no significant increase of IDS activity in the CNS exceeding the endogenous level of uninjected NOD.SCID mice, regardless of vector construct (data not shown). AAV9.hIDS was also injected into two groups of three wild type C57BL/6 (IDS+C57BL/6) mice at 8 weeks of age, one group via IT administration and the other group via IV administration. Again, no significant increase in the level of IDS activity in the CNS above the endogenous level of untreated controls was observed (data not shown). Thus, neither IT nor IV injection of IDS-encoding AAV vector appeared to be a suitable route of administration.

Another unexpected result from the initial pilot study described above is that while there was undetectable increase in IDS activity in the CNS, plasma IDS activity in both IV- and IT-treated groups was increased up to approximately 140-fold above the untreated wild-type level and persisted for at least 12 weeks post treatment. This result suggests that AAV vector was distributed to the peripheral circulation after IT injection into the cerebrospinal fluid (CSF). The presence of sustained enzyme activity for at least 12 weeks post injection (either IT or IV) also suggests that hIDS is presumably non-immunogenic for C57BL/6 mice.

The same four vector constructs (AAV9.hIDS, AAV9.hIDSco, AAV9.hIDShSUMF1, and AAV9.hIDSco-hSUMF1co) were administered into immunocompetent MPS II mice by ICV injection—a procedure that supports a much higher level of transduction in the CNS than IT injection (Wolf et al., 2011). Immunosuppression of the MPS II test animals was not necessary, since we found that expression of human IDS does not elicit an immune response in C57BL/6 mice. An additional group of MPS II mice was injected ICV with a combination of two vectors—AAV9.hIDS and AAV9.hSUMF1—at a 1:1 ratio (AAV9.hIDS+AAV9.hSUMF1; at a dose of 5×10¹⁰ vc total) to determine if there would be additional IDS activity when SUMF1 and IDS are both translated independently rather than relying on translation of SUMF1 from a downstream position by employing an IRES. Untreated wild-type littermates were used as controls. Six weeks post injection, the animals were euthanized, organs were harvested, and brains were microdissected to determine IDS activity. Animals injected with AAV9.hIDS, AAV9.hIDS-hSUMF1, or AAV9.hIDS+AAV9.h-SUMF1 showed levels of IDS activity approximately 10-40% of the wild-type level in most portions of the brain. IDS activity was undetectable in all areas of the brain in MPS II mice. Animals injected with codon-optimized vector constructs showed mostly <10% of the wild-type level, so codon optimization of the IDS sequence did not result in a higher level of IDS activity in transduced tissues. There was no significant difference between AAV9.hIDS-injected animals and animals injected with AAV9.hIDS plus hSUMF1. Thus, co-delivery of hSUMF1 either on the same vector or on a separate vector did not enhance the level of IDS activity assessed. Vector AAV9.hIDS was subsequently used for more extensive efficacy studies in ICV-administered MPS II mice, as described below, as neither the addition of SUMF1 nor the codon-optimization algorithm resulted in increased IDS activity compared to the native hIDS cDNA sequence.

Prevention of CNS and Peripheral Lysosomal Disease by ICV Administration of AAV9.hIDS Vector

A dose of 5.6×10¹⁰ AAV9.hIDS vc was infused into 8-week-old MPS II mice by ICV injection to achieve widespread CNS distribution of the vector through the CSF. As in the pilot study, plasma IDS activities up to 160-fold higher than wild type were observed in this larger cohort of ICV-treated MPS II animals, and this expression persisted throughout the experiment (28 weeks post injection).

Urine was collected at the end of the study (week 40 post injection) to evaluate the effect of long-term IDS expression on GAG excretion in the treated animals compared to wild-type and untreated MPS II mice. Urine GAG was significantly elevated in MPS II animals when compared to wild-type littermates (p<0.05). The treated animals demonstrated a significant reduction in urine GAG content (p<0.05) when compared to untreated littermates and were normalized when compared to the wild-type level (p>0.05).

At 10 months of age (40 weeks post injection), all mice were euthanized, and organs were harvested for analysis. IDS activity was undetectable in all areas of the brain and spinal cord of untreated MPS II mice. AAV9.hIDS-injected animals had IDS activity in all regions of the brain at approximately 9-28% of wild type, 53% in the olfactory bulb and 7% in the spinal cord. Although the vector was infused into the right ventricle of the brain, no significant difference in IDS activity was observed between the left and right hemispheres (p>0.05). Unlike the CNS, supraphysiological levels of enzyme activity were observed in all tested peripheral organs such as the heart, liver, spleen, and kidney (11-, 166-, 5-, and 3-fold, respectively), except in the lung where 34% of wild type was observed. This suggests that the vector was able to cross the BBB from the CNS into the circulation whereby it was taken up by and expressed in peripheral organs.

DNA was isolated from the same tissue homogenates and evaluated for vector distribution by qPCR. Consistent with Wolf et al. (2011), ICV infusion of AAV vector resulted in global distribution of vector in the CNS and in all of the tested peripheral tissues. The highest vc number was observed in the right hippocampus (49 vc/ge), while similar vc were observed between the left and right hemispheres for all regions of the brain. Unlike the CNS, relatively low copy numbers were detected in most tested peripheral tissues, including the heart, lung, spleen, and kidney of the treated animals (<0.6 vc/ge), while high vc numbers were observed in the liver (44 vc/ge). This suggests that enzyme produced by the liver was released into the circulation where the tested peripheral organs took up the circulating enzyme (i.e., metabolic crosscorrection).

The global distribution and expression of IDS had a significant effect on accumulation of lysosomal storage materials. Elevation of lysosomal GAG content was observed in the CNS of untreated MPS II mice when compared to wild-type littermates (p<0.0001). Even though there was only 10-40% of wild-type IDS level in the CNS, GAG content in the CNS was normalized when compared to wild type (p<0.01). Similar to the CNS, significant elevation of lysosomal GAG content was observed in all tested peripheral organs of the untreated MPS II animals when compared to wild type (p<0.01). In contrast, significantly decreased GAG levels were observed in all tested peripheral tissues of the treated mice when compared to the untreated group (p<0.01). Statistical analysis showed no significant difference in GAG content between wild-type animals and the treated group (p>0.05), which indicates that GAG content was normalized in the treated group.

The body weights of all mice were measured before sacrifice, and organs were weighed after the animals were perfused with 1×PBS, calculating the percentage of organ weight to body weight immediately post sacrifice. No significant difference was observed in the size of the heart, lung, spleen, or kidney among all groups. However, the liver of untreated MPS II mice was 20% larger than that of wild-type animals (6.2% and 5.2% of total body weight, respectively; p<0.001). In contrast, the liver of the treated MPS II mice was 68% smaller than the untreated group (4.2% and 6.2% of total body weight, respectively; p<0.0001). This result shows that normalization of GAG content in the liver in turn prevented hepatomegaly in the treated mice.

### Sustained Expression of IDS in the CNS Leads to Prevention of Neurocognitive Deficit in MPS II Mice

At 6 months of age (4 months post treatment), untreated MPS II mice, AAV9.hIDS-treated MPS II mice, and control normal littermates were evaluated for neurocognitive function in the Barnes maze, a test for spatial navigation and memory. The animals were subjected to a series of 3-minute trials, four trials per day for a course of 6 days. Wild-type littermates showed reduced latency to escape (30 s) on day 6, while untreated MPS II mice exhibited a significant deficit in learning this task (latency to escape reduced only to 71 s; p<0.05 vs. normal littermates). In contrast, the AAV9.hIDS-treated mice showed a marked reduction in latency to escape (25 s), significantly outperforming the untreated MPS II mice (p<0.01) on days 5 and 6. In addition, there was no significant difference between the treated animals and wildtype littermates (p>0.05). We conclude that sustained expression of IDS in the CNS prevented the emergence of neurocognitive deficits in MPS II mice when treated at a young age.

### Discussion

In this study, AAV9.hIDS using a strong promoter was administered to the CNS of MPS II mice. Levels of IDS activity in the CNS were only 7-28% of wild type. In contrast, levels of IDS activity in the circulation and in tested peripheral organs were at least 2- and up to 170-fold higher than wild-type levels. Sustained IDS expression leads to global normalization of GAG content. Finally, sustained levels of IDS activity had a profound effect on preventing neurologic deterioration.

No significant increase in IDS activity in the CNS above the endogenous level was observed in IV- or IT-treated IDS+ mice, regardless of vector construct, route of administration, or mouse strain, even though IDS was expressed under regulation of the strong CB7 promoter (data not shown). However, the level of plasma IDS activity in IT-treated IDS+ mice was an average of 100-fold higher than the wild-type level, indicating successful IT administration of vector. Similar high levels of plasma IDS were observed when AAV9. hIDS was infused into MPS II mice via ICV injection, although in this case IDS activity in the CNS above baseline was observed in all 12 assayed portions of the brain. Similar IDS activities were reported from Motas et al. (in coronal sections of the brain (Motas et al., 2016)) and Hinderer et al. (2016)(whole brain), in which they observed approximately 20-40% of wild-type levels in the CNS. Here, a more extensive analysis of vector distribution and expression is reported in all different micro-dissected areas of the brain after ICV administration of AAV9.hIDS in MPS II mice.

Unlike Motas et al. (2016) and Hinderer et al. (2016), there was limited level of IDS activity achieved in the CNS after ICV administration of AAV9.hIDS. These limited levels of IDS activity are in stark contrast with the levels of IDUA activity observed in the CNS of MPS I mice after ICV injection of AAV9-IDUA vector, in which 100- to 1,000-fold higher than wild type levels of IDUA activity are observed (Belur et al., 2014). Supraphysiological levels of IDS (>1,000 nmol/h/mg) were also observed in the liver of ICV-administered animals in our study at a similar vc number that yielded 10- to 100-fold less IDS expression (10-100 nmol/h/mg) in the CNS. Highly relevant to the goal of CNS-directed gene therapy for MPS II, therefore, is this question: what is it that limits expression of IDS in the brain after highly efficient AAV-mediated IDS gene delivery?

One possibility is that SUMF1 activity might be rate limiting for the generation of active IDS in the brain. SUMF1 is required for the post-translational activation of lysosomal sulfatases, including IDS (Sabourdy et al., 2015). hIDS enzyme clearly was activated in tissues of the MPS II mouse, presumably by mouse SUMF1, but this process could be rate limiting in the brain if AAV-encoded hIDS protein is expressed in a large quantity but only a limited amount of hIDS becomes activated. For example, Fraldi et al. (2007) demonstrated that N-sulfoglucosamine sulfohydrolase activities were increased when the enzyme was co-expressed with SUMF1 in their MPS IIIA studies. Anticipating a potential limitation of SUMF1, hIDS and hSUMF1 were co-transduced either on the same construct or on two separate vectors, but no significant increase in hIDS activity was found compared to delivery of hIDS alone. hIDS activity was not enhanced by co-delivery with hSUMF1 in these experiments.

Another possibility is that the CB7 promoter might be limiting when compared to the endogenous IDS promoter. To further investigate this possibility, IDS activity (nmol/h/mg protein=unit) per vc was calculated for each tissue and compared to IDS activity per endogenous copy in male mice. IDS activities in the CNS of the treated mice were observed between 2 and 32 (mean of 8) units per vc compared to wild-type male mice, which express an average of 200 units per genome equivalent (approximately only 1-31% of wild-type level). By comparison, in the liver there was an average of 55 units of IDS activity per vc in the ICV-treated mice. From this, one might conclude that the CB7 promoter is not as robust as the endogenous IDS promoter in the brain. However, the lower level of vector-mediated IDS expression per vc versus endogenous expression most likely results from the presence of excess transcriptionally inactive AAV genomes remaining in the intracranial space post injection, possibly due to inefficient intracellularization. Nonetheless, the MPS II results contrast greatly with the results from our MPS I studies in which levels of IDUA activity at approximately 10- to 100-fold higher than wild type were observed in the CNS of mice administered AAV-hIDUA intrathecally (Belur et al., 2014), while heterozygous MPS I animals express approximately 6 units per genome equivalent in the brain (Ou et al., 2014). Therefore, it is feasible to achieve or exceed wild-type IDUA levels in the CNS of MPS I mice. The promoter that we used in the MPS II study was similar but not identical to the promoter used in the MPS I studies (Wolf et al., 2011; Belur et al., 2014), and the possibility that relative promoter strength may have contributed to the observed differences in outcome cannot be excluded.

Surprisingly, supranormal levels of enzyme activity were observed in all tested peripheral organs of the treated animals. The highest levels of IDS activity (164-fold wild type) were observed in the liver, which correlated with the highest number of vc found (48 vc/ge). In contrast, the tested peripheral organs (other than the liver) contained low levels of vector, but levels were higher than wild-type levels of IDS activity. The exceptionally high levels of IDS in the liver lead to sustained high levels of IDS activity (up to 172-fold higher than wild type) in the circulation. Similar results have been reported in a MPS IIIA study and several MPS I studies using different types of vectors (Aronovich et al., 2009; Osborn et al., 2011; Haurigot 2013), along with two studies in MPS II mice (Motas et al., 2016; Hinderer et al., 2016). These results suggest that in the present study, the liver acts as an enzyme factory that produces an enormous amount of IDS and releases it into the circulatory system where it is subsequently taken up by the other organs in the periphery. Even though higher than wild-type levels of IDS enzyme activity were observed in the heart, spleen, and kidney in the treated mice, the vector levels in these organs were unexpectedly low (<0.6 vc/ge). This low transduction rate suggests that these organs took up circulating IDS, leading to metabolic cross-correction with higher levels of IDS than wild type.

Polito et al. observed no detectable IDS activity in the CNS after a single IV injection of AAV2/5 CMV-hIDS. However, they did observe partial correction of GAG content in the CNS (Polito et al., 2009). They speculated that only a minute fraction of high-level circulating hIDS produced from the liver penetrated the BBB into the CNS, with subsequent partial correction of GAGs. Similarly, in the current experiments exceptionally high levels of IDS were found in the circulation of AAV9.hIDS-treated mice, and yet only subphysiological activities of enzyme were observed in the CNS. This finding indicates that an insignificant amount of enzyme produced from the liver after ICV injection of vector was able to penetrate the BBB from the circulation into the CNS. This is consistent with the lack of increased IDS over wild-type levels in the CNS after IV administration of AAV9.hIDS in IDS+C57BL/6 mice. Therefore, the level of IDS observed in the brain most likely relied on transduction of the vector construct inside the CNS after ICV injection rather than from circulating enzyme. Further investigation is needed to achieve IDS levels comparable to or higher than the wild type in the CNS.

Sustained levels of enzyme expression after direct vector injection into the CNS have been shown to have profound effects on GAG reduction in several MPS studies irrespective of whether wild-type levels of those enzymes were achieved (Janson et al., 2014; Barnes, 1979; Belur et al., 2014; Motas et al., 2016; Ou et al., 2014). Similarly, although only subphysiological levels of IDS were observed in the CNS after ICV injection of AAV9.hIDS, there was a significant effect of the enzyme on GAG reduction that was greater than expected. Desnick et al. and Polito et al. speculated that only <5% of the wild-type level of lysosomal enzyme is needed to correct a GAG storage defect (Polito et al., 2009; Desnick et al., 2012). The present results are consistent with this speculation. After AAV9.hIDS treatment, all areas of the brain and even the spinal cord (the organ with the lowest enzyme measured; 10.7 nmol/h/mg protein; 7.4% of wild type) showed significantly lower GAG content than in untreated MPS II mice. Statistical analysis revealed striking results, as GAG content in the CNS of the treated mice was normalized when compared to wild type. We also observed normalization of GAG content in the urine and in the heart, lung, liver, spleen, and kidney of the treated animals. These results indicate a sustained effect of IDS expression on correction of GAG content.

Roberts et al. demonstrated a direct relationship between GAG accumulation and liver size when injecting rodamine B, a GAG synthesis inhibitor, into MPS IIIA mice. They found that GAG content was decreased in the liver, leading to normalization of liver size (Roberts et al., 2006). Motas et al. observed a preventive effect on hepatomegaly after ICV administration of AAV9.hIDS into MPS II mice (Motas et al., 2016). Similarly, we also observed that the weight of the liver in treated mice was normalized after ICV injection of AAV9.hIDS. This result indicates a profound effect of sustained IDS expression leading to normalization of GAG content in the liver, which in turn prevents hepatomegaly in the treated mice.

Several MPS studies have demonstrated prevention of neurologic deficits after AAV-mediated gene transfer in mice (Wolf et al., 2011; Motas et al., 2016; Fu et al., 2011). Neurocognitive deficiency in untreated MPS II mice was observed at 6 months of age. We also observed that sustained IDS expression in the CNS prevented the emergence of neurocognitive dysfunction in MPS II mice after ICV infusion of AAV9.hIDS. Several studies have shown that the hippocampus is associated with neurocognition in rodents (Seeger et al., 2004; Paylor et al., 2001; Miyakawa et al., 2001). However, the possibility that physical impairment such as vision, olfactory sense, or motor neuron defects could also affect the results in the Barnes maze cannot be excluded, as rodents require all of the aforementioned physical capabilities in order to perform the required task in this test (Harrison et al., 2006). Additional behavioral analyses would support the observation that neurocognitive deficit plays a pivotal role in learning impairment of untreated MPS II mice and its prevention by AAV-mediated IDS gene transfer.

In conclusion, the present application characterizes the benefit of direct AAV9-mediated hIDS gene transfer to the CNS. The most important challenge emerging from this study is the limited level of AAV-mediated IDS expression achieved in the CNS in comparison with other tissues such as the liver, and in comparison with the expression of other therapeutic genes introduced into the CNS by AAV-mediated transduction such as IDUA (Wolf et al., 2011; Belur et al., 2014). Nonetheless, the MPS II data indicate that direct injection of AAV9.hIDS vector into the CNS resulted in efficient gene transfer that is key to treatment of MPS II and prevention of neurocognitive deficits. We also found that AAV9.hIDS vector was capable of crossing the BBB from the CNS into the circulation, resulting in global transduction of the vector outside of the CNS, providing long-term expression of IDS enzyme systemically. Even though the IDS activities in the brain were lower than expected, this study nonetheless supports the notion from previous studies (Polito et al., 2009; Desnick et al., 2012) that <10% of the wild-type level of IDS is needed to prevent GAG storage accumulation. In addition, sustained IDS expression corrected the accumulation of GAG in liver and subsequently prevented the emergence of hepatomegaly. Finally, the results reinforce the importance of sustained IDS expression in the CNS in preventing the emergence of neurologic deficits when animals are treated at a young age.

### Example IV

Mucopolysaccharidosis type I (MPS I) is an inherited autosomal recessive metabolic disease caused by deficiency of α-L-iduronidase (IDUA), resulting in accumulation of heparin and dermatan sulfate glycosaminoglycans (GAGs). Individuals with the most severe form of the disease (Hurler syndrome) suffer from neurodegeneration, mental retardation, and death by age 10. Current treatments for this disease include allogeneic hematopoietic stem cell transplantation (HSCT) and enzyme replacement therapy (ERT). However, these treatments are insufficiently effective in addressing CNS manifestations of the disease.

The goal is to improve therapy for severe MPS I by supplementing current ERT and HSCT with IDUA gene transfer to the CNS, thereby preventing neurological manifestations of the disease. In this study, the ability of intravenously administered AAV serotypes 9 and rh10 (AAV9 and AAVrh10) to cross the blood brain barrier for delivery and expression of the IDUA gene in the CNS was tested. 4-5 month old adult MPS I animals were infused intravenously via the tail vein with either an AAV9 or AAVrh10 vector encoding the human IDUA gene. Blood and urine samples were collected on a weekly basis until the animals were sacrificed at 10 weeks post-injection. Plasma IDUA activities in treated animals were close to 1000-fold higher than that of heterozygote controls at 3 weeks post-injection. Brains, spinal cords, and peripheral organs were analyzed for IDUA activity, clearance of GAG accumulation, and IDUA immunofluorescence of tissue sections. Treated animals demonstrated widespread restoration of IDUA enzyme activity in all organs including the CNS. These data demonstrate the effectiveness of systemic AAV9 and AAVrh10 vector infusion in counteracting CNS manifestations of MPS I.

### Example V

Gene transfer offers enormous potential for therapy of the mucopolysaccharidoses. Studies have focused on achieving high-level expression of alpha-L-iduronidase (IDUA) in the CNS of MPS I mice, where it was observed enzyme up to 1000-fold greater than wild-type (WT) in the brain after intracerebroventricular (ICV) infusion of AAV9 transducing the human IDUA gene. Intrathecal (IT) infusion of AAV9 vector also resulted in high-level IDUA expression (10- to 100-times that of wild-type) throughout the brain. All routes of administration normalized glycosaminoglycan levels in all areas of the brain and prevented the emergence of neurocognitive deficiency at 4-5 months of age as assessed in the Barnes maze. WT mice expressed much higher levels of endogenous iduronate sulfatase (IDS) than IDUA in the brain, and in animals infused IT with AAV9 transducing the human IDS gene, the level of IDS in the brain was indistinguishable from WT. After ICV infusion of AAV9-IDS vector in MPS II mice there was sufficient expression of IDS to reduce GAG accumulation to near wild-type levels and prevent the emergence of neurocognitive dysfunction, but the level of IDS never achieved that of WT in the brain.

### Example VI

Hunter Syndrome (Mucopolysaccharidosis type II; MPS II) is an X-linked recessive inherited lysosomal disease caused by deficiency of iduronate-2-sulfatase (IDS) and accumulation of glycosaminoglycans (GAGs) in tissues, resulting in skeletal dysplasias, hepatosplenomegaly, cardiopulmonary obstruction, and neurologic deterioration. Patient standard of care is enzyme replacement therapy (ERT) although ERT is not associated with neurologic improvement. In a mouse model of IDS deficiency, intracerebroventricular (ICV) administration of AAV9.hIDS into young 8-week old mice resulted in corrective levels of hIDS enzyme activity, reduction of GAG storage to near WT-levels and prevention of neurocognitive dysfunction, compared to IDS deficient control littermates. Since the emergence of neurologic manifestations could be prevented in young adults, it was hypothesized that older adult MPS II animals treated at 4 months of age by ICV administration of AAV9.hIDS would recover neurobehavioral function and show corrected levels of IDS enzyme activity and GAG storage. By 4 weeks post-ICV injection, IDS enzyme activity in the circulation was 1000-times that of WT-levels (305+/−85 nmol/hr/ml compared to 0.39+/−0.04 nmol/hr/ml). At 36 weeks of age, the treated animals were tested for neurocognitive function in the Barnes maze. Performance of the treated animals was indistinguishable from that of unaffected littermates and significantly improved compared to untreated MPS II mice. Cognitive function that is lost by 4 months of age can thus be restored in MPS II mice by delivery of AAV9 encoding IDS to the cerebrospinal fluid. The implication of these results is the prospect that human MPS II may be treatable after the development of neurologic manifestations by AAV mediated IDS gene transfer to the CNS.

### Example VII

MPSII is a rare X-linked lysosomal storage disease. MPSII patients have a deficiency in IDS and accumulate GAGs Clinical manifestations include coarse facial features, short stature, dysostosis multiplex, joint stiffness, skeletal dysplasias and spinal cord compression, organomegaly, retinal degeneration, cardiac/respiratory obstruction, pebbled skin and intellectual disability (severe form). Current treatments (HSCT and ERT) are lacking in that in HSCT there is a very low level of enzyme expression in HSCT (MPSI) and so it is less likely to provide a benefit in reversing neurological deficit, and in ERT enzymes are rapidly depleted and do not cross blood brain barrier, and no neurological improvement.

In order to investigate whether treatment with AAV-IDS of older MPSII animals that already manifested neurological deficit has a beneficial effect, 4 month old MPSII mice were ICV administered AAV-hIDS.

MPSII animals treated at 4 months by AAV9.hIDS ICV injection exhibited 500×WT IDS enzyme activity in plasma, about 100×WT IDS enzyme activity in liver and elevated enzyme activity in the brain, e.g., hippocampus about 1/3 WT levels, and GAG levels restored to WT levels in all tissues, and treatment restored neurocognitive function. The invention will be described by the following non-limiting examples.

### Example A

The lysosomal enzyme alpha-L-iduronidase (IDUA) catalyzes degradation of glycosaminoglycans (GAGs) heparan- and dermatan-sulfate. Absence of functional IDUA is the etiology of MPSI. MPSI patients demonstrate lysosomal accumulation of GAG; leading to a multisystemic, chronic, and progressive disease, e.g., patients evidence growth delay, organomegaly, cardiopulmonary disease, skeletal dysplasia and neurocognitive decline. Patients with the severe form of disease do not survive beyond age 10.

Enzyme replacement therapy for MPSI is not effective for some manifestations of the disease because administered enzyme does not cross the blood brain barrier and so there is no correction of neurologic or residual somatic disease burden. Hematopoietic cell transplant (HCT) does result in donor cells with normal levels of IDUA crossing the blood-brain barrier. This, in turn, prevents neurocognitive decline and is the treatment of choice for severe MPSI. However, there is wide variability in correction of neurological disease by HCT, and below normal IQs and impaired neurocognitive ability can occur despite successful HCT. Regardless of treatment, cardiac valve disease, aortic root dilation and skeletal dysplasia remain largely untreated and are current areas of unmet need.

To evaluate the benefits of systemic versus CNS-directed routes of AAV9-IDUA vector administration on the neurological and systemic manifestations of MPS I in a mouse model, the following experiments were conducted in order to determine if sustained and therapeutic levels of functional IDUA enzyme activity was achieved both systemically and in the CNS.

In this study, 2-month old MPS I mice were divided into 3 groups and administered 10 vector genomes of an AAV9 based vector transducing the human IDUA sequence (AAV9-IDUA), either intrathecally, intravenously, or in a combination of both routes. Following injection, IDUA levels in plasma were consistently 100-(females) to 1000-(males)-fold higher than normal heterozygote levels in all 3 treatment groups. Mice were euthanized at 5-6 months post-treatment, followed by tissue assays for IDUA and GAG levels. Untreated male MPS I mice reliably exhibited cardiac defects that effectively model manifestations that are observed in human MPS I, including reduced ejection fraction, dilation of the ascending aorta and aortic insufficiency. Prior to sacrifice, treated animals were evaluated by high resolution echocardiography for the effect of AAV9-IDUA administration on the murine cardiac disease in comparison with untreated MPS I control animals. Infusion with AAV9-IDUA largely alleviated the cardiac defects exhibited by male MPS I mice, particularly for animals administered vector intravenously. Specifically, the infusion largely prevented development of aortic dilation and aortic insufficiency while maintaining normal cardiac systolic function. These data are supported by demonstration of restored IDUA activity and normalization of GAG storage in the heart for animals administered AAV9-IDUA, particularly for animals administered AAV vector intravenously. The results of this study support the anticipated clinical benefit of treatment using AAV-IDUA vector, administered intravenously, intrathecally, or by both routes of administration, to remedy the cardiac disease suffered in human MPS I.

BM measures spatial memory and navigation which is a function of the hippocampus. The animal is trained to locate an escape hole on the maze, and time taken to locate the escape hole is recorded as latency to escape. The test is carried out over 4 days with 4 trials per day, and as you can see, it takes them less time to locate the escape hole on day 4 than on day 1.

Fear Conditioning (FC) is a function of associative learning that is primarily associated with the hippocampus, but also other parts of the brain such as the amygdala. In this test, fear response (freezing) was measured after administration of a mild foot shock

Cardiac function in treated and MPS I mice was evaluated using echocardiography (ECHO). Cardiac systolic function was maintained in treated animals. Most treated mice had normalized ascending aortic diameters compared to untreated MPS I controls, especially IV administered group. Most treated MPSI mice had no aortic insufficiency in contrast to age-matched controls.

Thus, administration of IDUA-encoding AAV can ameliorate cardiac manifestations of the disease that remain unmet by current treatment strategies. This sets AAV-mediated gene transfer apart from currently employed ERT and HSCT in addressing cardiac defects of MPS I and the prospect for improved patient outcomes in managing the cardiac manifestations of MPS I and other MPS diseases.

### Summary

Supraphysiological and sustained levels of IDUA were found in plasma following all routes of administration DUA activity in the plasma, brain, and liver was higher in males than in females. IDUA activities were highest in the brain after IT delivery; neurocognitive function was restored in all treated groups. Normal cardiac function was observed after IT, IV and IV+IT delivery routes in comparison to untreated MPSI mice. Ascending aortic dilation and aortic valve insufficiency were largely prevented in treated MPSI males. Normal to high levels of IDUA activity were found in all major organs, normalization of GAG accumulation after all 3 routes of administration.

Thus, the methods provide for a remedy for peripheral manifestations (cardiac and skeletal) of LSDs not only after IV administration of AAV vector but even after AAV delivery into the CSF, e.g., intrathecally. It means that much of the vector is released from the CSF into the circulation, with subsequent transduction of peripheral tissues from which enzyme is secreted and disseminated.

### Example B

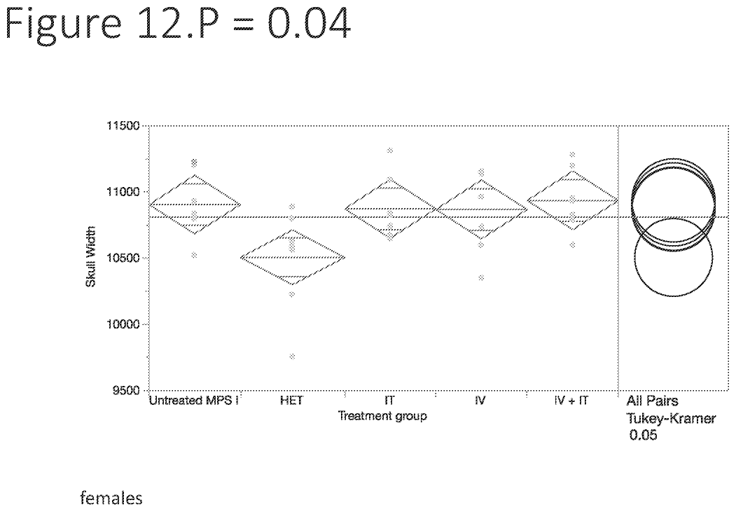

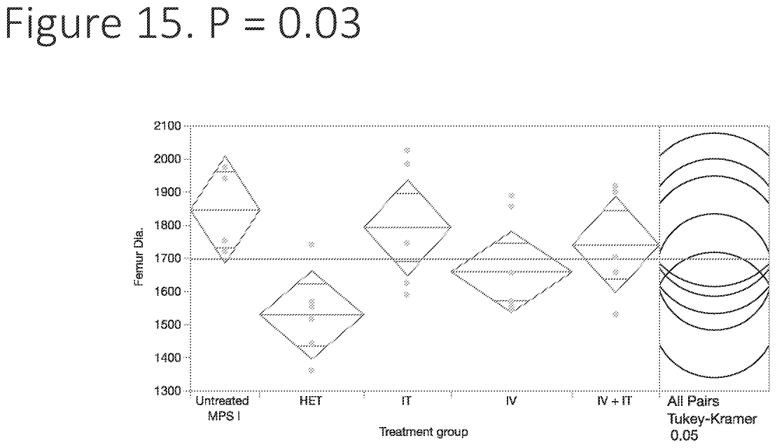

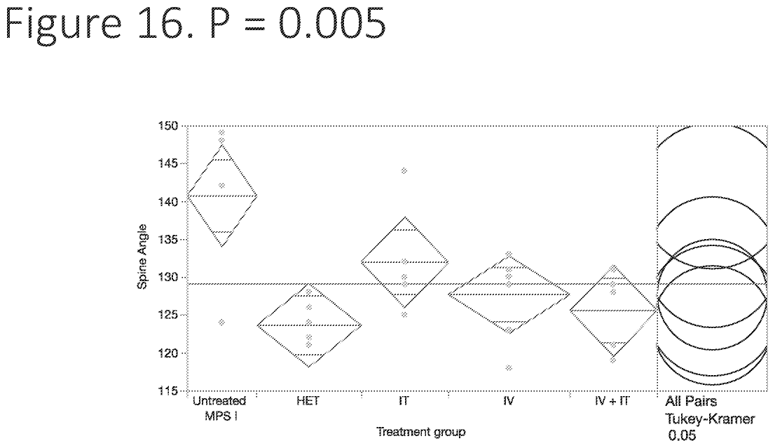

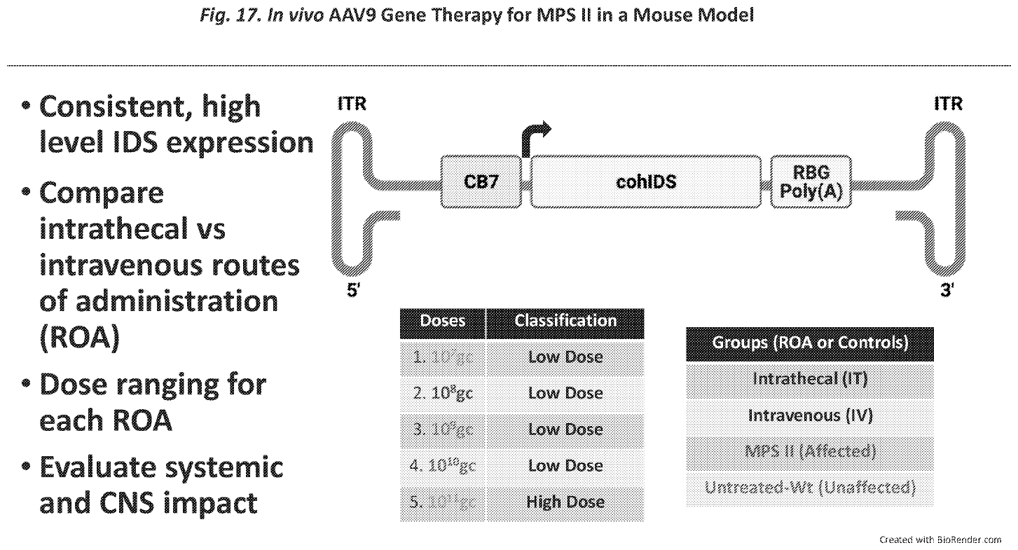

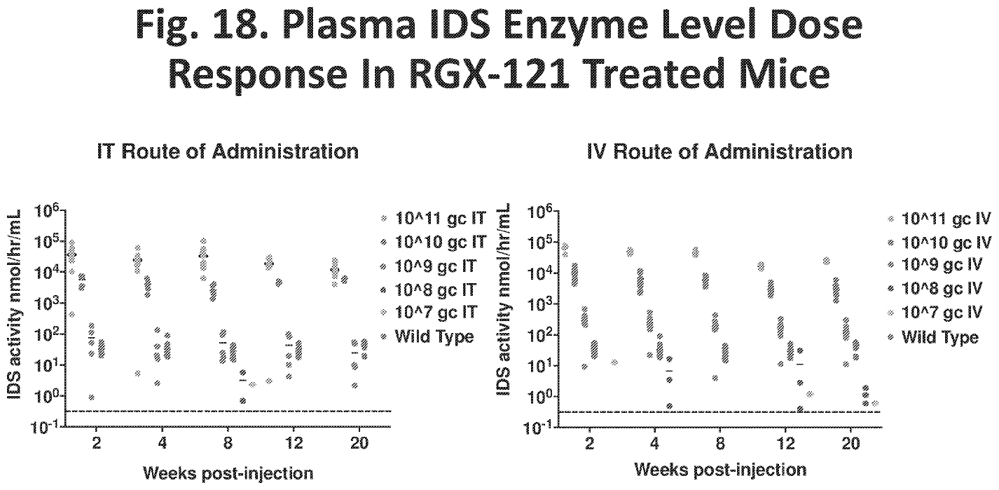

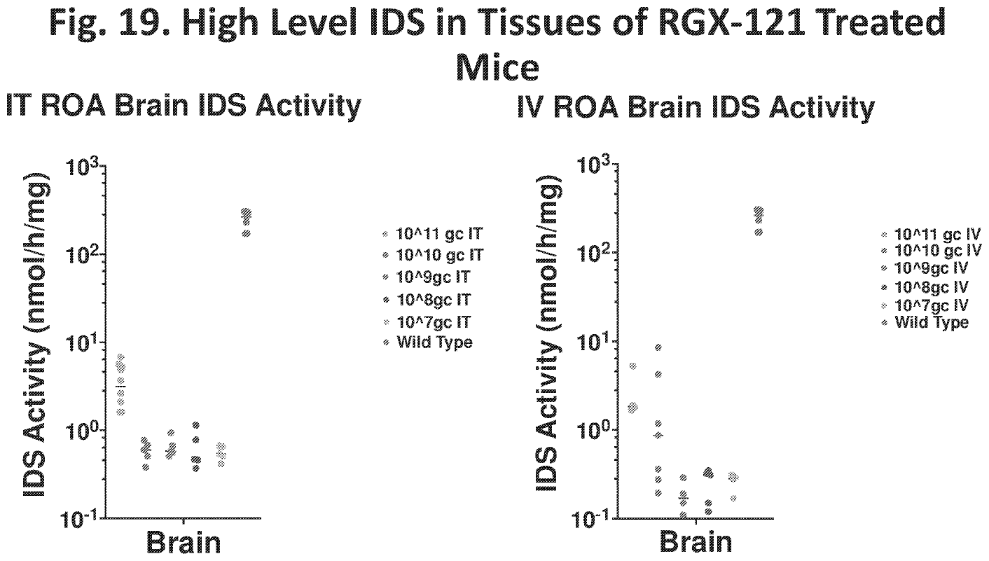

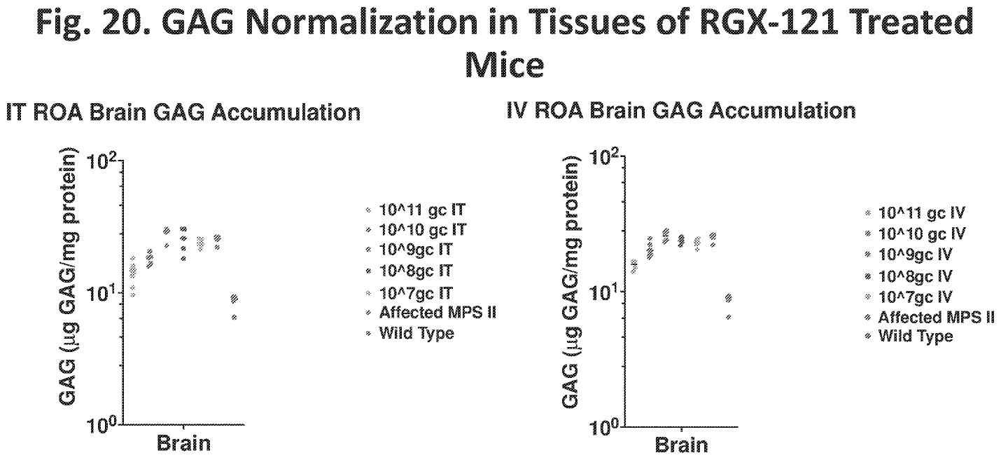

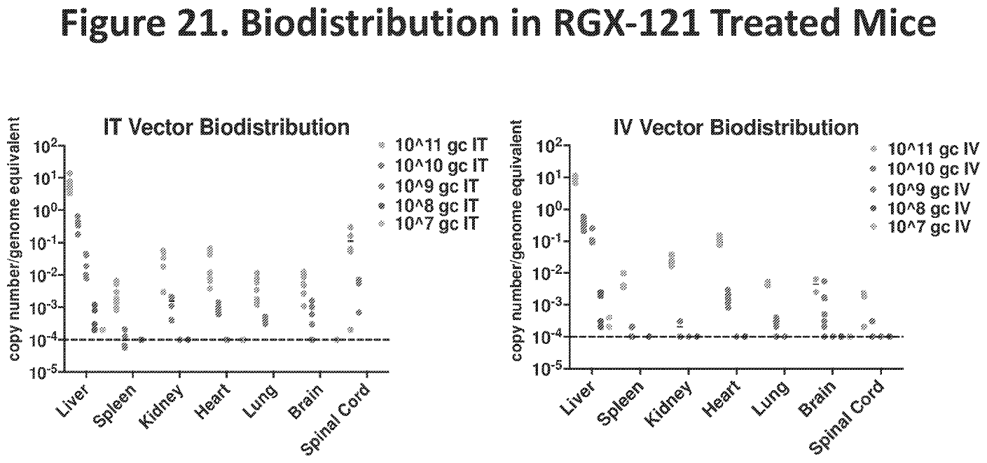

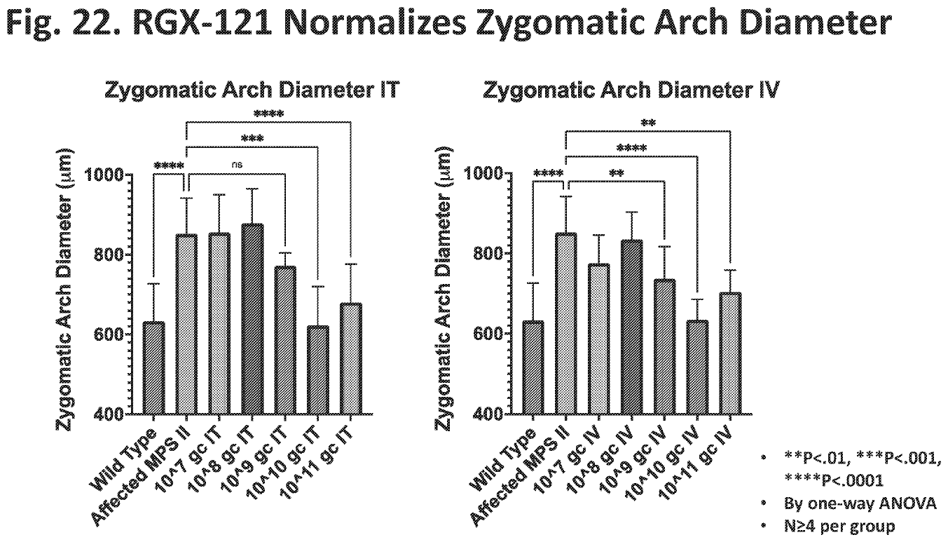

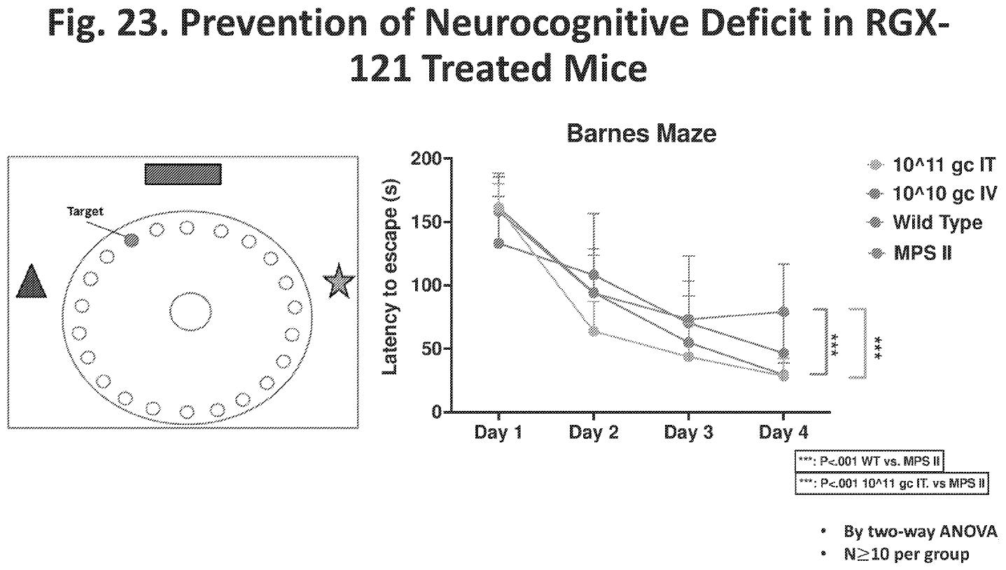

A three-dimensional image of the entire skeleton of mice in Example A was obtained by computed tomography (“microCT”) of the animals at end of study, before euthanization (FIGS. 12-16). Then, after the animal is euthanized a tibia is dissected out, stripped of tissue, and subjected to biomechanical and bone morphometry analyses (essentially three-point breakage and focused microCT testing).

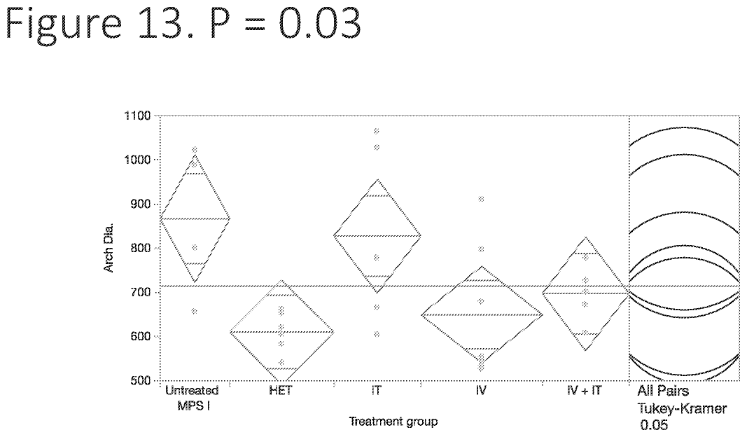

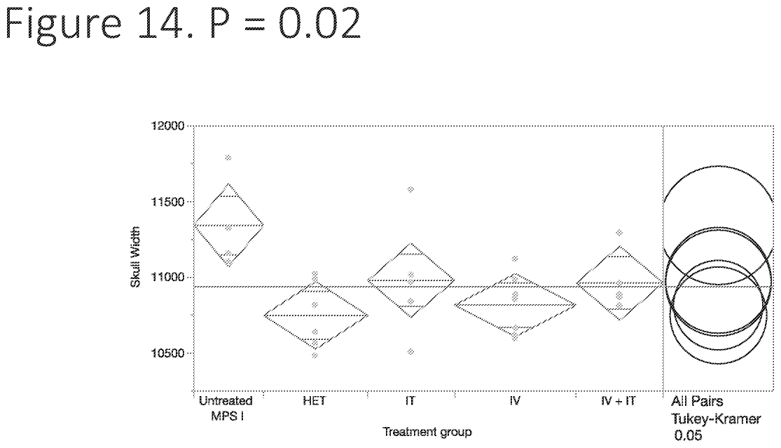

MPS I mice are known to have a wider skull width (see FIGS. 12 and 14, whole body CT). The females do not exhibit a change from the untreated MPS I animals, but the males show normalization of skull width (FIG. 14). FIG. 13 shows improvement in zygomatic arch in the males that have been treated with AAV-IDUA vector IV, but less so for animals given vector IT. Femur diameter shows improvement particularly for the IV-treated males, and spine angle is markedly improved for both IV and IT treated males. This latter observation is noteworthy because kyphosis is a known manifestation of MPS I in humans, here remedied by administration of AAV-IDUA IV or IT. The biomechanical analyses show no improvement in cortical thickness or cortical bone mineral density, but improvements in cross-sectional moment of inertia (MOM) in MPS I animals administered AAV9-IDUA IV or IT and in bone stiffness for MPS I mice administered AAV9-IDUA IT.

### Example C

Mucopolysaccharidosis type I (MPS I) is an autosomal recessive storage disease caused by deficiency of α-L-iduronidase (IDUA), resulting in accumulation of heparan and dermatan sulfate glycosaminoglycans (GAGs). Current treatments include allogeneic hematopoietic stem cell transplantation (HSCT) and enzyme replacement therapy (ERT). However, ERT is ineffective against CNS disease due to the inability of lysosomal enzymes to traverse the blood-brain barrier, and while there is neurologic benefit to HSCT, the level of correction is variable, and the procedure is associated with morbidity and mortality. Preclinical studies of IDUA gene therapy using AAV vectors have provided encouraging results for the treatment of MPS I.

In this study, the relative efficacy of intrathecal, intravenous, and combined routes of AAV vector administration on systemic, neurologic, cardiac, and skeletal manifestations of disease were investigated. It was also determined if there was a benefit to adding intravenous delivery of AAV9-IDUA to intrathecal administration. AAV9-IDUA was administered at the same total dose (1×10¹⁰ vg) either intrathecally (IT), intravenously (IV), or in a combination of both routes (IT+IV) to 2-month old MPS I mice. The animals were euthanized at 6 months post-treatment, and then tissues were harvested and assayed for IDUA activity and GAG levels. IDUA levels in plasma, brain and liver showed a gender-related effect, with a 10-fold higher level seen in males. Plasma IDUA levels were 100-1000-fold higher than normal in all 3 groups. Enzyme activities in the brain were highest after IT administration (10-fold higher than normal), with lowest levels seen after IV administration. Supraphysiological levels of IDUA in liver were seen for all three groups (100-1000-fold higher than normal). The three test groups yielded similar levels of enzyme activity in all other organs, and GAG levels were normalized or reduced in all groups. IV, IT or IV+IT administration of AAV9-IDUA prevented the emergence of neurocognitive deficit exhibited in MPS I mice, with no significant difference between the treatment groups. Cardiac valve function analysis by high resolution ultrasound biomicroscopy showed aortic insufficiency (AI) in most untreated MPS I mice, while the IT+IV group showed no aortic insufficiency, and the IT and IV groups had only 1 mouse in each group with AI. The ascending aortic diameter was normalized in the IV (all mice) and the IT/IT+IV groups (1 mouse had increased diameter), compared to untreated MPS I mice. Skeletal analysis of vector-treated mice by microCT showed normalization of the skull width, zygomatic arch diameter, and kyphosis in male mice. Cross sectional moment of inertia was also normalized in both male and female treated mice. Our results show that AAV9-IDUA vector, administered IV, IT or both, resulted in high levels of enzyme activity in major organs, and all three treatments were effective in preventing neurocognitive deficit, cardiac valve dysfunction and skeletal dysplasias in MPS I mice.

Thus, the methods disclosed in Examples A-C, demonstrating that rAAV(s) can be employed to prevent cardiac, vascular or skeletal defects, or alter disease progression of those defects, for MPSI, may be employed in lysosomal diseases other than MPS1, e.g., MPSII, MPSIII, MPSIV, MPSVI, MPSVII and the like.

### Example D

Exemplary Human Doses of a rAAV for IT or IC Administration

IT/IC administration of rAAV in the absence of administration of rAAV by another route may be from about 1×10¹⁰ GC/g brain to 2×10¹¹ GC/g brain. The amount delivered IT/IC may result in systemic delivery, e.g., up to about 85% of the dose may be in the blood, which in turn leads to an improvement in cardiac symptoms. In one embodiment, the dose may be 1.3×10¹⁰ GC/g brain, 6.5×10¹⁰ GC/g brain, or 2×10¹¹ GC/g brain. In one embodiment, the human has MPSII and is administered rAAV-IDS at a dose of 1.3×10¹⁰ GC/g brain, 6.5×10¹⁰ GC/g brain, or 2×10¹¹ GC/g brain. In one embodiment, the dose may be 1.3×10¹⁰ GC/g brain or 5×10¹⁰ GC/g brain. In one embodiment, the human has MPSI and is administered rAAV-IDUA at a dose of 1.3×10¹⁰ GC/g brain or 5×10¹⁰ GC/g brain.

Exemplary Human Doses of a rAAV for IV Administration

IV administration of rAAV in the absence of administration of rAAV by another route may be from 1×10¹¹ GC/kg of body weight to 1×10¹² GC/kg of body weight. In particular, the dose may be about 1×10¹¹ GC/kg, 2×10¹¹ GC/kg, 3×10¹¹ GC/kg, 4×10¹¹ GC/kg, 5×10¹¹ GC/kg, 6×10¹¹ GC/kg, 7×10¹¹ GC/kg, 8×10¹¹ GC/kg, 9×10¹¹ GC/kg, or 1×10¹² GC/kg. In one embodiment, when other routes of administration are not in the best interests of the patient, IV administration alone may be employed. For example, as children with MPS I and MPS II get older (over 3 years old) they have increased intracranial pressure and so there is a risk in administering IT/IC in these patients because that administration can herniate the brain. IV dosing may provide for treatment of cardiac and/or skeletal defects, which are the leading cause of death in MPS patients. IV dosing may also allow for some transduction in the brain and so there may be improvement of CNS symptoms as well, e.g., relative to the absence of treatment.

Exemplary Human Doses of a rAAV for IT/IC and for IV Administration

IT/IC administration of rAAV, in combination with administration of rAAV by another route, may be from about 1×10¹⁰ GC/g brain to 2×10¹¹ GC/g brain. The IT dose may be 1.3×10¹⁰ GC/g brain, or 5×10¹⁰ GC/g brain, 6.5×10¹⁰ GC/g brain, or 2×10¹¹ GC/g brain. When the other route is IV administration, the dose of rAAV may be from 1×10¹¹ GC/kg of body weight to 1×10¹² GC/kg of body weight. The IV dose may be about 1×10¹¹ GC/kg, 2×10¹¹ GC/kg, 3×10¹¹ GC/kg, 4×10¹¹ GC/kg, 5×10¹¹ GC/kg, 6×10¹¹ GC/kg, 7×10¹¹ GC/kg, 8×10¹¹ GC/kg, 9×10¹¹ GC/kg, or 1×10¹² GC/kg. The combined effect of the dual routes of administration may show more improvement in cardiac and/or skeletal defects, e.g., relative to the absence of treatment, because at least some of the IC/IT dose, and the IV dose, reach the heart and skeletal tissues. Thus, the combination of IT/IC and IV may be curative for cardiac and skeletal defects.

In one embodiment, the IT/IC and IV doses are given on the same day or within one, two or three days, or more, e.g., 2 to 4 weeks, of each other. Close timing of the two doses may avoid an anti-AAV Nabs surge within the first 2 weeks after gene therapy is administered. In one embodiment, the IV dose is administered first and the IT/IC dose is administered 2 to 4 weeks later.

### Exemplary Immunosuppressive Regimen

One or more immune suppressants may be employed with administration of the rAAV(s) described herein. For example, a corticosteroid, mTOR inhibitor, e.g., a macrolide such as rapamycin (sirolimus), a calcineurin inhibitor that may be a macrolide such as tacrolimus (FK-506), anti-thymocyte globulin, a T cell depleting agent, or a T cell inhibitor such as cyclosporin or mycophenolate mofetil, may be employed.

The immune suppressant may be administered before the rAAV, e.g., earlier on the same day or for 1-2 days before rAAV delivery, or both, and then optionally for days or weeks thereafter. For instance, a corticosteroid, for example, methylprednisolone at 10 mg/kg IV (maximum of 500 mg), is administered the day of the rAAV administration and subsequently an oral formulation of a corticosteroid may be delivered for up to 12 to 14 weeks. In one embodiment, on day 2, oral prednisone therapy is started with a goal to discontinue prednisone by week 12. The dose of prednisone may be as follows: Day 2 to the end of Week 2: 0.5 mg/kg/day; Week 3 and 4: 0.35 mg/kg/day; Week 5-8: 0.2 mg/kg/day; Week 9-12: 0.1 mg/kg

The immune suppressant may be administered before the rAAV, e.g., for 1-2 days before rAAV delivery, and then optionally for days or weeks thereafter. For instance, a macrolide that is a mTOR inhibitor such as sirolimus is administered for 2 days prior to vector administration (day −2), e.g., a loading dose of sirolimus is administered at about 1 mg/m2 every 4 hours×3 doses, and then on day −1: sirolimus is administered at about 0.5 mg/m2/day divided in twice a day dosing with a target blood level of about 1-3 ng/ml. Sirolimus may be discontinued after week 48.

The immune suppressant may be administered only after the rAAV is administered, e.g., later on the same day or beginning at 1-2 days after rAAV delivery, or both, and then optionally for days or weeks thereafter. For instance, a macrolide that is a calcineurin inhibitor such as tacrolimus is administered starting on day 2 at a dose of about 0.05 mg/kg twice daily and adjusted to achieve a blood level of about 2-4 ng/mL for up to about 24 weeks. The dose may then be decreased by approximately 50% and optionally at week 28 the dose is further decreased by approximately 50% with a goat to discontinue tacrolimus by week 32.

The doses and regimens described above may be employed to prevent cardiac, vascular or skeletal defects, or alter disease progression of those defects, for a variety of lysosomal diseases including but not limited to MPS1, e.g., MPSII, MPSIII, MPSIV, MPSVI, MPSVII and the like.

### Example E

Treatment of Cardiac, Neurologic, and Skeletal Manifestations of Murine MPS I with AAV9-IDUA: Efficacy Study of Vector Dose and Route of Administration

Mucopolysaccharidosis type I (MPS 1) is caused by deficiency of alpha-L-iduronidase (IDUA). Current treatments for MPS I do not sufficiently address the manifestations of this disease. To assess the most effective route and dose of AAV9-IDUA vector administered to MPS I mice, 2-month old MPS I mice were treated with an AAV9-IDUA vector (doses ranging from 10⁷-10¹⁰ vector genomes, vg) administered via the following routes: (i) intrathecal (IT), (ii) intravenous (IV), and (iii) intrathecal+intravenous (IT+IV). Mice were analyzed for cardiac, neurologic, and skeletal improvements 4 months after treatment. In mice administered doses ≤10⁹ vg IT or ≤10⁸ vg IV of AAV9-IDUA, there was no detectable IDUA activity in plasma or tissues of mice, nor was there normalization of GAG, nor correction of neurocognitive or cardiac defects. In mice administered 10⁹ vg IV, there was an increase in IDUA activity in plasma and in tissues ranging from normal to 100 times normal with neurocognitive and cardiac assessments inconclusive at this dose. However, at the highest vector dose (10¹⁰ vg), substantial biochemical correction with restored IDS activity and normalized GAG were observed in all treatment groups. Cardiac valve functions were mostly normalized across treatment groups, and there was prevention of neurologic deficit observed in all treated animals. Skeletal analysis by microCT showed normalization of skull width, zygomatic arch diameter, and kyphosis in IV and IT+IV treated male MPS I mice, with trending towards normal in IT treated mice. Histologic analysis corroborated the biochemical data and showed minimal storage based on Alcian Blue staining. Overall, these results characterized a minimal effective dose of 10¹⁰ vg for both IV and IT routes of administration, alone or in combination, for preventing neurocognitive deficit and in improving cardiac and skeletal manifestations of murine MPS I, which may be applicable to human MPS I.

### Example F

Comparative Effectiveness of Intravenous and Intrathecal AAV9.hIDS (RGX-121) in a Murine Model of Mucopolysaccharidosis Type II

The only currently approved treatment for Mucopolysaccharidosis type II (MPSII) is enzyme replacement therapy (ERT). However, ERT does not address the neurocognitive and behavioral disease manifestations. AAV9.hIDS (RGX-121) administered intrathecally is an alternative strategy to overcome this limitation and treat the neurological manifestations of MPSII. It is unknown if intrathecal administration of AAV9.hIDS will also improve systemic manifestations of MPSII or whether supplemental systemic administration will be required. A CNS directed (intrathecal) route of administration (ROA) was compared with systemic (intravenous) administration at varying doses in order to determine if intrathecal dosing of AAV9.hIDS improves systemic and neurological outcomes in a murine model of MPSII. While plasma IDS activities at or above wild type were observed in animals administered 10⁹ vc AAV9-IDS by either ROA, this dose was insufficient to achieve either wild type IDS activity or reduced glycosaminoglycans (GAG) in most tissues, including the CNS. However, at doses of 10¹⁰ vc and 10¹¹ vc, both IT and IV, plasma IDS activity reached >100-1000 times the wild type level and tissues showed at or above wild type levels of IDS activity with a commensurate normalization of GAG content. Administration of 10¹¹ vc IT was required to achieve quantifiable levels of IDS activity in the brain, a dose that also prevented the emergence of neurocognitive deficits. Varying levels of GAG reduction in the brain were observed in animals administered either 10¹⁰ vc or 10¹¹ vc AAV9.hIDS via either route of administration. Significant reduction in zygomatic arch diameter was observed in animals administered 10¹⁰ vc or 10¹¹ vc (IT and IV) AAV9.hIDS compared to untreated controls or animals administered vector at lower doses. Thus, IT administration of AAV9.hIDS improved the neurologic, metabolic and skeletal disease in MPSII mice indicating a somatic benefit of IT dosing for the treatment of MPSII in humans.

### Example G

Treatment of Cardiac, Neurologic, and Skeletal Manifestations of Murine MPS I with AAV9-IDUA: Efficacy Study of Vector Dose and Route of Administration

Mucopolysaccharidosis type I (MPS I) is caused by deficiency of alpha-L-iduronidase (IDUA). Current treatments for MPS I do not sufficiently address the manifestations of this disease. Our goal was to assess the most effective route and dose of AAV9-IDUA vector administered to MPS I mice. 2-month old MPS I mice were treated with an AAV9-IDUA vector (doses ranging from 10⁷-10¹⁰ vector genomes, vg) administered via the following routes: (i) intrathecal (IT), (ii) intravenous (IV), and (iii) intrathecal+intravenous (IT+IV). Mice were analyzed for cardiac, neurologic, and skeletal improvements 4 months after treatment. In mice administered doses ≤10⁹ vg IT or ≤10⁸ vg IV of AAV9-IDUA, there was no detectable IDUA activity in plasma or tissues of mice, nor was there normalization of GAG, nor correction of neurocognitive or cardiac defects. In mice administered 10⁹ vg IV, there was an increase in IDUA activity in plasma and in tissues ranging from normal to 100 times normal with neurocognitive and cardiac assessments inconclusive at this dose. However, at the highest vector dose (10¹⁰ vg), substantial biochemical correction was observed with restored IDS activity and normalized GAG in all treatment groups. Cardiac valve functions were mostly normalized across treatment groups, and there was prevention of neurologic deficit observed in all treated animals. Skeletal analysis by microCT showed normalization of skull width, zygomatic arch diameter, and kyphosis in IV and IT+IV treated male MPS I mice, with trending towards normal in IT treated mice. Histologic analysis corroborated the biochemical data and showed minimal storage based on Alcian Blue staining. Overall, these results characterized a minimal effective dose of 10¹⁰ vg for both IV and IT routes of administration, alone or in combination, for preventing neurocognitive deficit and in improving cardiac and skeletal manifestations of murine MPS I, which may be applicable to human MPS I.

### Example H

Mucopolysaccharidosis II (MPS II, Hunter syndrome) is a recessive X-linked disorder (˜1:160,000 males) caused by deficiency of Iduronate-2-sulfatase (IDS) enzyme activity, and is a progressive, multisystemic disorder. Hunter syndrome is characterized by glycosaminoglycans (GAGs) accumulation skeletal dysplasias, organomegaly, airway obstruction, and neurocognitive decline (severe). The current approved treatment is. enzyme replacement therapy.

A mouse MPSII model was employed to determine the effect of gene therapy (RGX-121). In the untreated B6-MPSII mice, there was extensive neuropil vacuolization in the medulla (green arrows). In these same brain regions, there was progressively decreased vacuolization in the IV-AAV and IT-AAV treated mice, which was more pronounced in the IT-treated mice as compared to the IV-treated mice.

In summary, the results showed low to undetectable IDS enzyme and little GAG reduction in animals administered very low doses (10⁷ gc, 10⁸ gc) of RGX-121, supraphysiological levels of plasma IDS enzyme in animals administered RGX-121 IT (10¹⁰ gc, 10¹¹ gc) or IV (10⁹ gc, 10¹⁰ gc, 10¹¹ gc), normalized urine GAG excretion in animals administered RGX-121 IT (10¹⁰ gc, 10¹¹ gc) or IV (10⁹ gc, 10¹⁰ gc, 10¹¹ g, 10¹⁰ gc IV was minimally sufficient for significant metabolic correction and normalization of zygomatic arch diameter, and 10¹¹ gc IT was necessary to achieve measurable IDS activity and GAG reduction in the CNS, with prevention of neurocognitive deficit in the Barnes maze.

### Example I

Comparative Effectiveness of Intravenous and Intrathecal AAV9.CB7.hIDS (RGX-121) in a Murine Model of Mucopolysaccharidosis Type II

The only currently approved treatment for Mucopolysaccharidosis type II (MPSII) is enzyme replacement therapy (ERT). However, ERT does not address the neurocognitive and behavioral disease manifestations of MPSII. AAV9.CB7.hIDS (RGX-121, AAV9 vector encoding human iduronate-2-sulfatase) administered intrathecally (IT) is an alternative strategy to treat the neurological manifestations of MPSII. It is unknown if IT administration of AAV9.CB7.hIDS will also improve systemic manifestations of MPSII or whether supplemental systemic administration will be required. We set out to compare a CNS-directed (IT) route of administration (ROA) with systemic (intravenous; IV) administration at varying doses. Our goal was to determine if IT dosing of AAV9.CB7.hIDS improves systemic and neurological outcomes in a murine model of MPSII. While plasma IDS activity at or above wild type levels was observed in animals administered 1.0×10⁹ vg AAV9.CB7.hIDS by either ROA, this dose was insufficient to achieve either wild type IDS activity or reduce glycosaminoglycans (GAGs) in most tissues, including the CNS. However, at doses of 1.0×10¹⁰ vg and 1.0×10¹¹ vg, both IT and IV, plasma IDS activity reached >100-1000 times the wild type levels, and tissues showed at or above wild type levels of IDS activity with a commensurate normalization of GAGs. Administration of 1.0×10¹¹ vg IT was required to achieve quantifiable levels of IDS activity in the brain, a dose that also prevented the emergence of neurocognitive deficits. Varying levels of GAGs reduction in the brain were observed in animals administered either 1.0×10¹⁰ vg or 1.0×10¹¹ vg AAV9.CB7.hIDS via either route of administration. Significant reduction in zygomatic arch diameter was observed in animals administered 1.0×10¹⁰ vg or 1.0×10¹¹ vg AAV9.CB7.hIDS compared to untreated controls or animals administered vector at lower doses. Thus, IT administration of AAV9.CB7.hIDS improved the neurologic, metabolic and skeletal disease in MPSII mice.

In conclusion, there were low to undetectable IDS enzyme and little GAG reduction in animals administered very low doses (10⁷ gc, 10⁸ gc) of RGX-121. Supraphysiological levels of plasma IDS enzyme were observed in animals administered RGX-121 IT (10¹⁰ gc, 10¹¹ gc) or IV (10⁹ gc, 10¹⁰ gc, 10¹¹ gc). Normalized urine GAG excretion was found in animals administered RGX-121 IT (10¹⁰ gc, 10¹¹ gc) or IV (10⁹ gc, 10¹⁰ gc, 10¹¹ gc). Thus, 10¹⁰ gc IV was minimally sufficient for significant metabolic correction and normalization of zygomatic arch diameter, while 10¹¹ gc IT was necessary to achieve measurable IDS activity and GAG reduction in the CNS, with prevention of neurocognitive deficit in the Barnes maze.

### Example J

The most effective route of RGX-111 administration to MPS I animals (IT/IV/IT+IV) and the minimal effective dose of RGX-111 vector (10⁷, 10⁸, 10⁹, 10¹⁰ vg) were evaluated.

### Route of Administration (10¹⁰ VG)

INTRATHECAL (IT)

| | | | | | | | |
|---|---|---|---|---|---|---|---|
| Animal | Co- | | | Treat- | Volume, | Injec- | Eutha- |
| # | hort | DOB | Gender | ment | Dose | tion | nasia |

| | | | | |
|---|---|---|---|---|
| cl5031 | RC4 | M | IT | 10 ul, 1e10 |
| 5032 | RC4 | M | IT | 10 ul, 1e10 |
| 5033 | RC4 | M | IT | 10 ul, 1e10 |
| 5034 | RC4 | F | IT | 10 ul, 1e10 |
| 5035 | RC4 | F | IT | 10 ul, 1e10 |
| 5036 | RC4 | F | IT | 10 ul, 1e10 |
| 5273 | RC6 | N | IT | 10 ul, 1e10 |
| 5274 | RC6 | F | IT | 10 ul, 1e10 |
| 5327 | RC7 | F | IT | 10 ul, 1e10 |
| 5328 | RC7 | F | IT | 10 ul, 1e10 |
| 5429 | RC7 | M | IT | 10 ul, 1e10 |
| 5430 | RC7 | M | IT | 10 ul, 1e10 |
| 5607 | RC10 | M | IT | 10 ul, 1e10 |
| 5611 | RC10 | F | IT | 10 ul, 1e10 |

INTRAVENOUS (IV)

| | | | | | | | |
|---|---|---|---|---|---|---|---|
| Animal | Co- | | | Treat- | Volume, | Injec- | Eutha- |
| # | hort | DOB | Gender | ment | Dose | tion | nasia |

| | | | | |
|---|---|---|---|---|
| 5258 | RC5 | M | IV | 100 ul, 1e10 |
| 5259 | RC5 | M | IV | 100 ul, 1e10 |
| 5260 | RC5 | M | IV | 100 ul, 1e10 |
| 5261 | RC5 | M | IV | 100 ul, 1e10 |
| 5262 | RC5 | F | IV | 100 ul, 1e10 |
| 5263 | RC5 | F | IV | 100 ul, 1e10 |
| 5264 | RC5 | F | IV | 100 ul, 1e10 |
| 5441 | RC8 | F | IV | 100 ul, 1e10 |
| 5442 | RC8 | F | IV | 100 ul, 1e10 |
| 5443 | RC8 | F | IV | 100 ul, 1e10 |
| 5339 | RC8 | M | IV | 100 ul, 1e10 |
| 5340 | RC8 | M | IV | 100 ul, 1e10 |
| 5341 | RC8 | M | IV | 100 ul, 1e10 |
| 5608 | RC10 | M | IV | 100 ul, 1e10 |
| 5612 | RC10 | F | IV | 100 ul, 1e10 |

INTRATHECAL (IT) + INTRAVENOUS (IV)

| | | | | | | | |
|---|---|---|---|---|---|---|---|
| Animal | | | | Treat- | Volume, | Injec- | Eutha- |
| # | Cohort | DOB | Gender | ment | Dose | tion | nasia |

| | | | | |
|---|---|---|---|---|
| 5266 | RC6 | M | IV + IT | 1.00E+10 |
| 5267 | RC6 | M | IV + IT | 1.00E+10 |
| 5268 | RC6 | M | IV + IT | 1.00E+10 |
| 5270 | RC6 | F | IV + IT | 1.00E+10 |
| 5271 | RC6 | F | IV + IT | 1.00E+10 |
| 5272 | RC6 | F | IV + IT | 1.00E+10 |
| 5439 | RC8 | M | IV + IT | 1.00E+10 |
| 5440 | RC8 | M | IV + IT | 1.00E+10 |
| 5501 | RC9 | M | IV + IT | 1.00E+10 |
| 5496 | RC9 | F | IV + IT | 1.00E+10 |
| 5497 | RC9 | F | IV + IT | 1.00E+10 |
| 5498 | RC9 | F | IV + IT | 1.00E+10 |
| 5626 | RC10 | M | IV + IT | 1.00E+10 |
| 5629 | RC10 | F | IV + IT | 1.00E+10 |

UNTREATED MPS I

| | | | | | | | |
|---|---|---|---|---|---|---|---|
| Animal | Co- | | | Treat- | Volume, | Injec- | Eutha- |
| # | hort | DOB | Gender | ment | Dose | tion | nasia |

| | | | | |
|---|---|---|---|---|
| 5038 | RC4 | M | Untreated | No Vector |
| 5039 | RC4 | F | Untreated | No Vector |
| 5072 | RC4 | F | Untreated | No Vector |
| 5011 | RC4 | M | Untreated | No Vector |
| 5012 | RC4 | M | Untreated | No Vector |
| 5010 | RC4 | F | Untreated | No Vector |
| 5265 | RC5 | F | Untreated | No Vector |
| 5269 | RC6 | M | Untreated | No Vector |
| 5275 | RC6 | F | Untreated | No Vector |
| 5431 | RC7 | M | Untreated | No Vector |
| 5432 | RC7 | M | Untreated | No Vector |
| 5320 | RC7 | F | Untreated | No Vector |
| 5628 | RC10 | M | Untreated | No Vector |
| 5631 | RC10 | F | Untreated | No Vector |

HETEROZYGOTE

| | | | | | | | |
|---|---|---|---|---|---|---|---|
| Animal | Co- | | | Treat- | Volume, | Injec- | Eutha- |
| # | hort | DOB | Gender | ment | Dose | tion | nasia |

| | | | | |
|---|---|---|---|---|
| 5353 | RC7 | F | Untreated | No Vector |
| 5324 | RC7 | F | Untreated | No Vector |
| 5326 | RC7 | F | Untreated | No Vector |
| 5356 | RC8 | F | Untreated | No Vector |
| 5438 | RC8 | F | Untreated | No Vector |
| 5518 | RC9 | M | Untreated | No Vector |
| 5519 | RC9 | M | Untreated | No Vector |
| 5489 | RC9 | M | Untreated | No Vector |
| 5482 | RC9 | M | Untreated | No Vector |
| 5483 | RC9 | M | Untreated | No Vector |
| 5512 | RC9 | F | Untreated | No Vector |
| 5609 | RC10 | M | Untreated | No Vector |
| 5610 | RC10 | F | Untreated | No Vector |
| 5613 | RC10 | F | Untreated | No Vector |

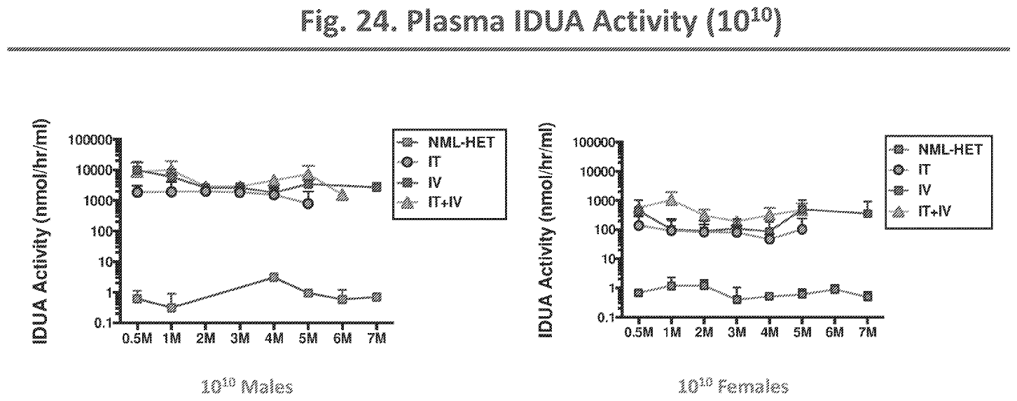

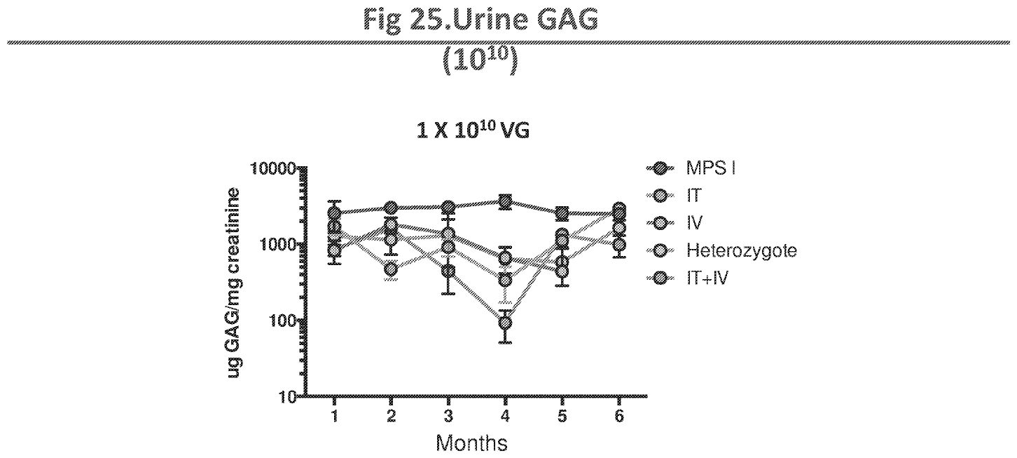

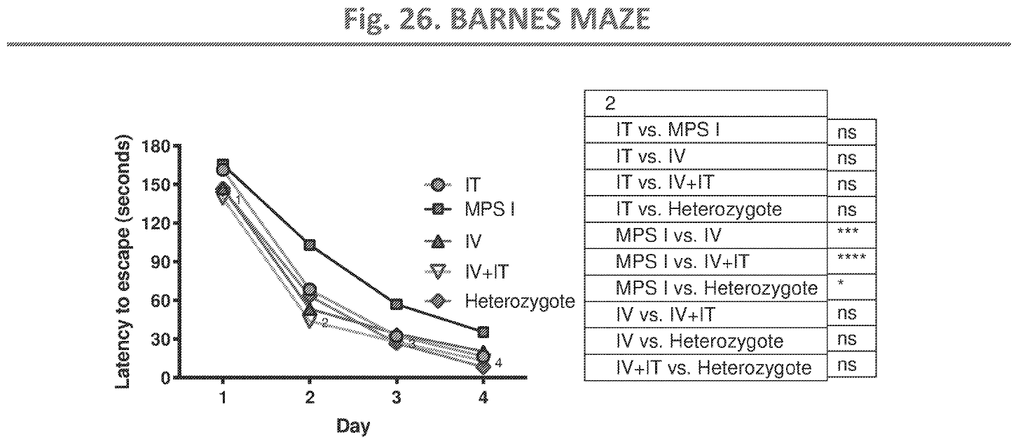

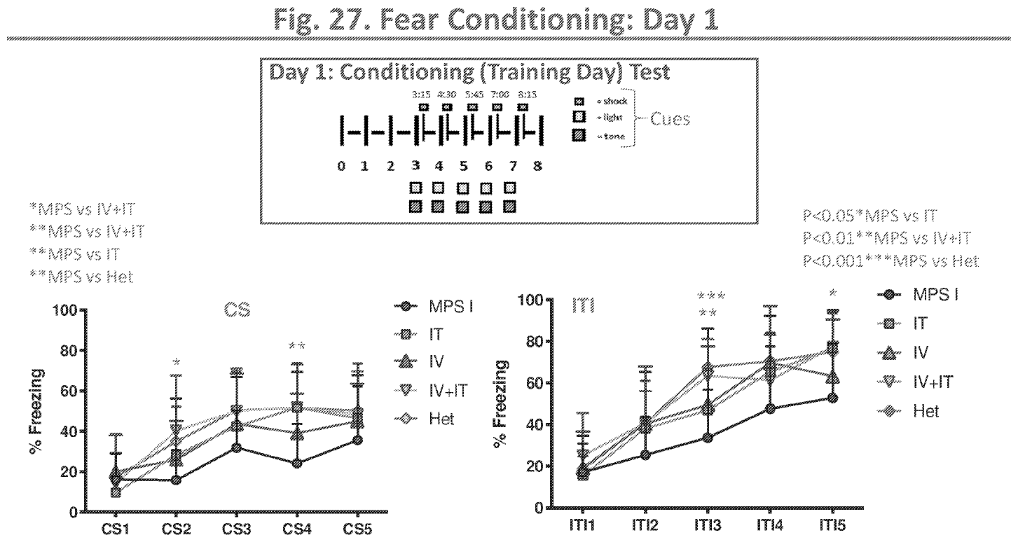

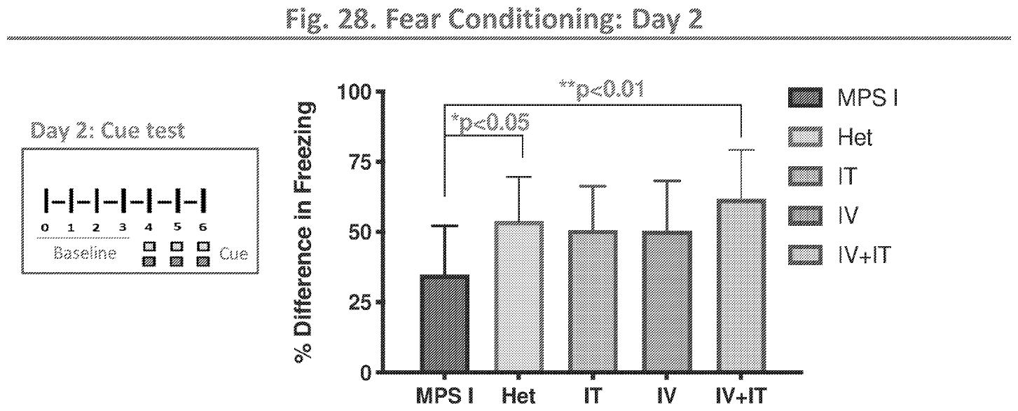

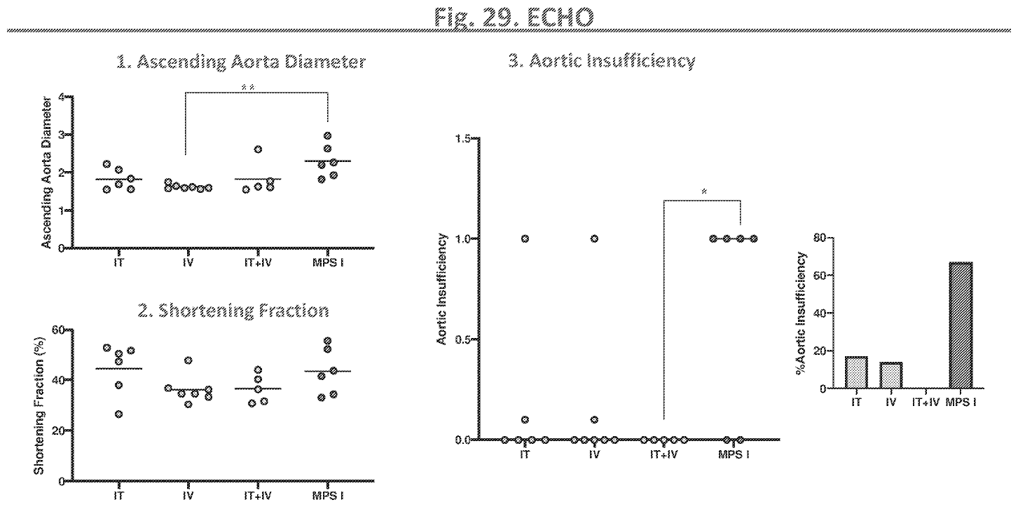

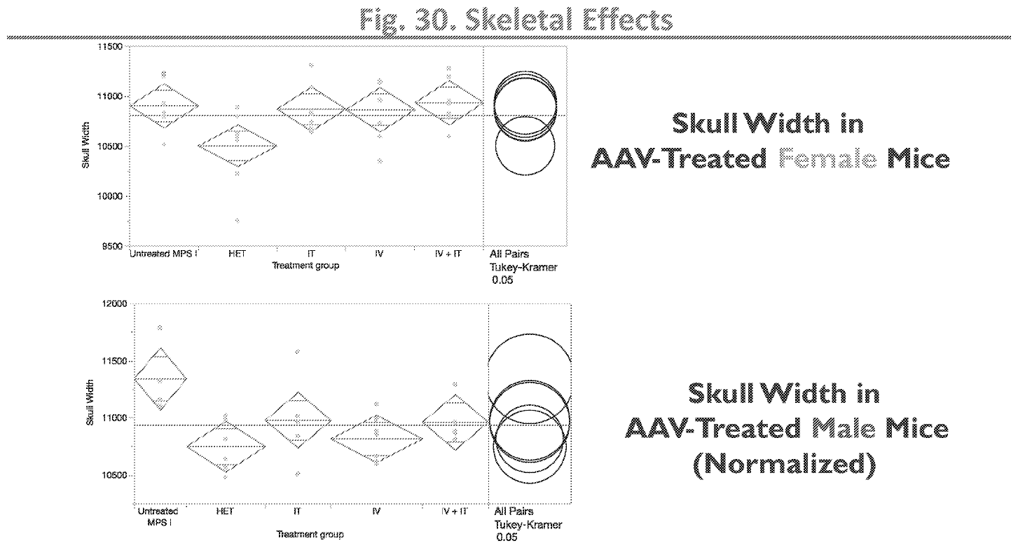

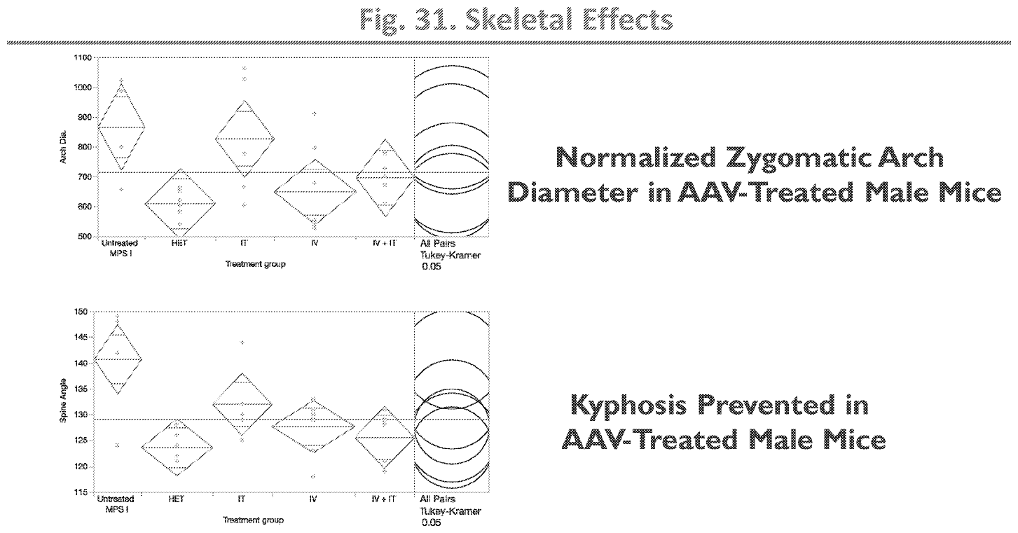

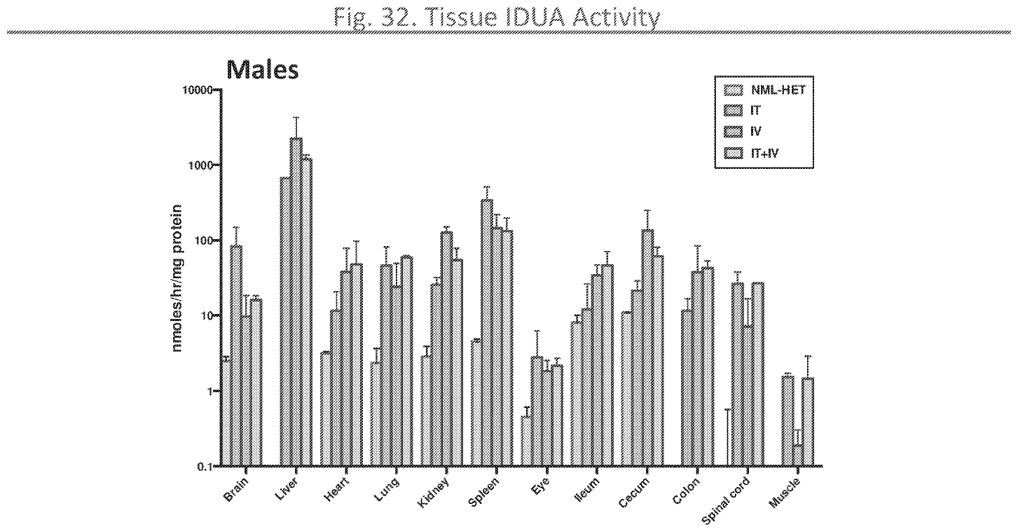

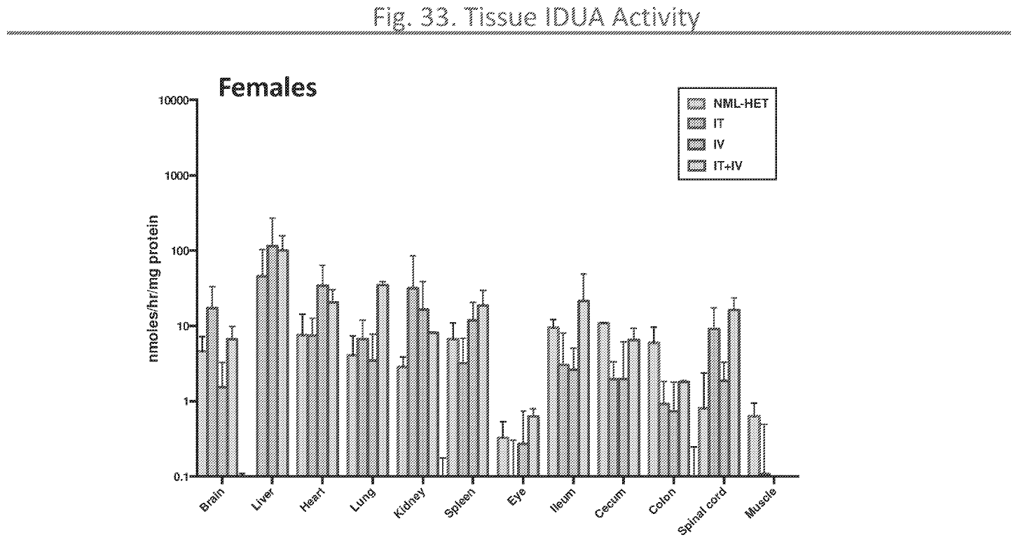

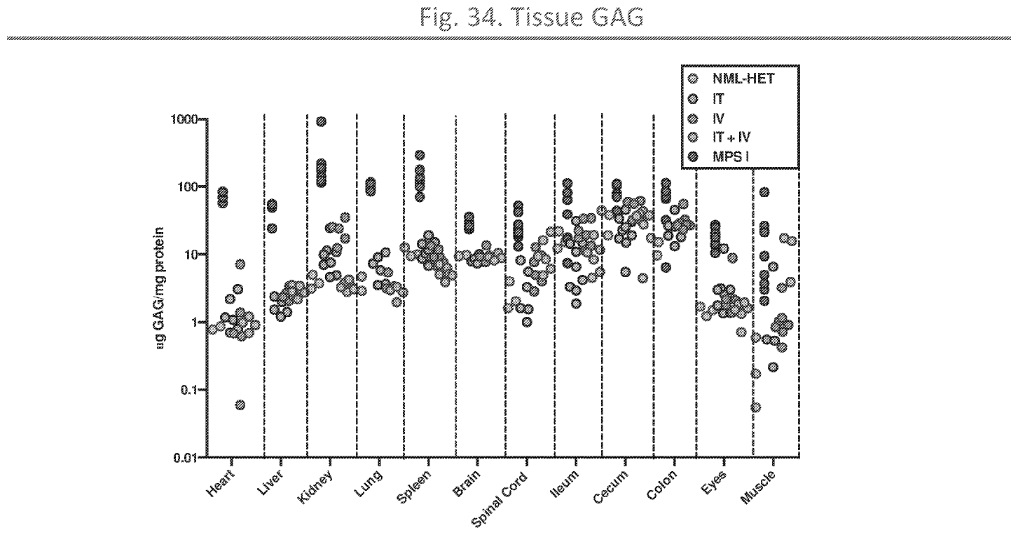

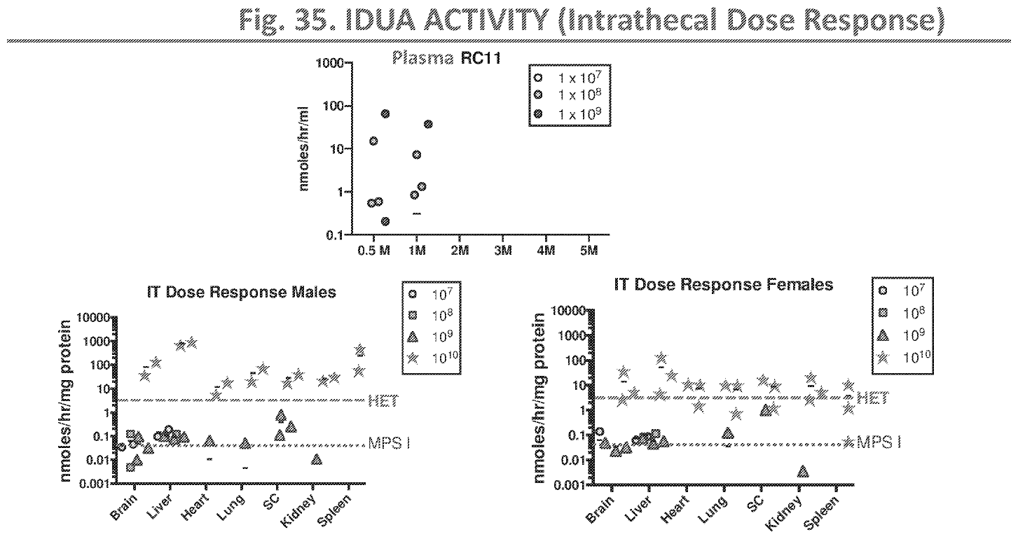

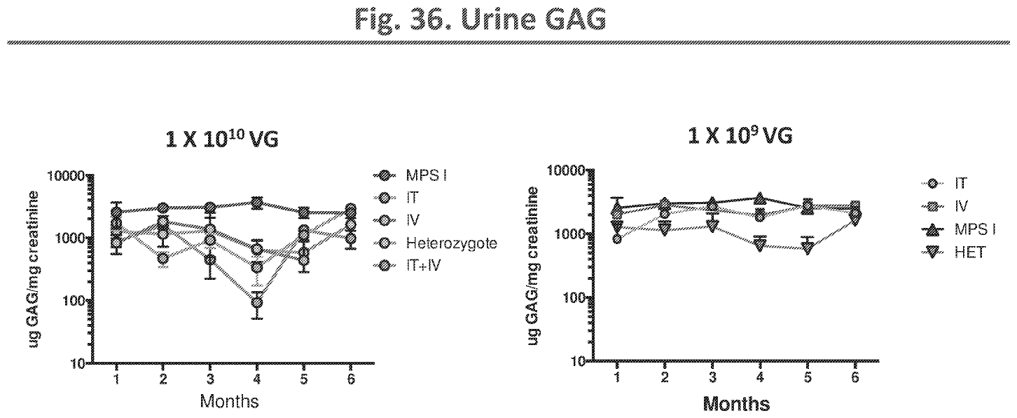

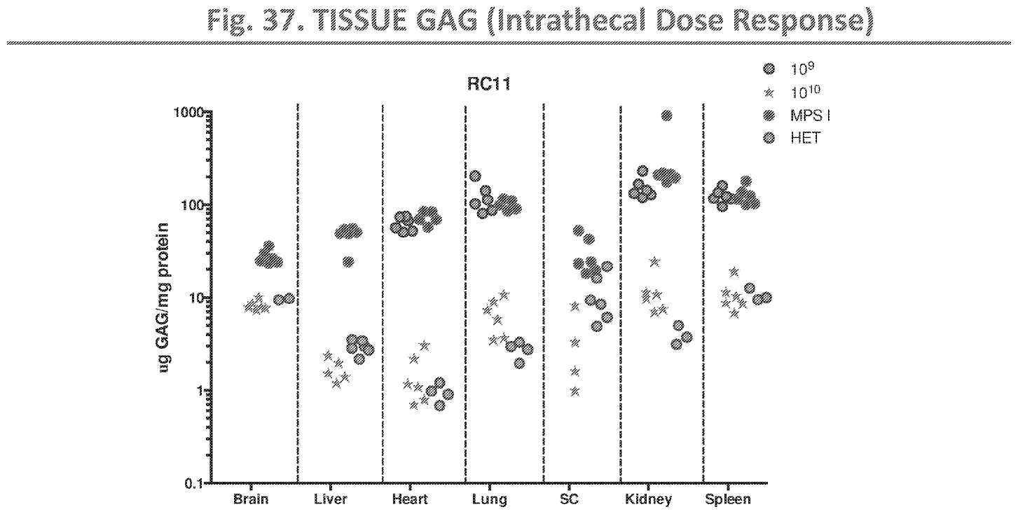

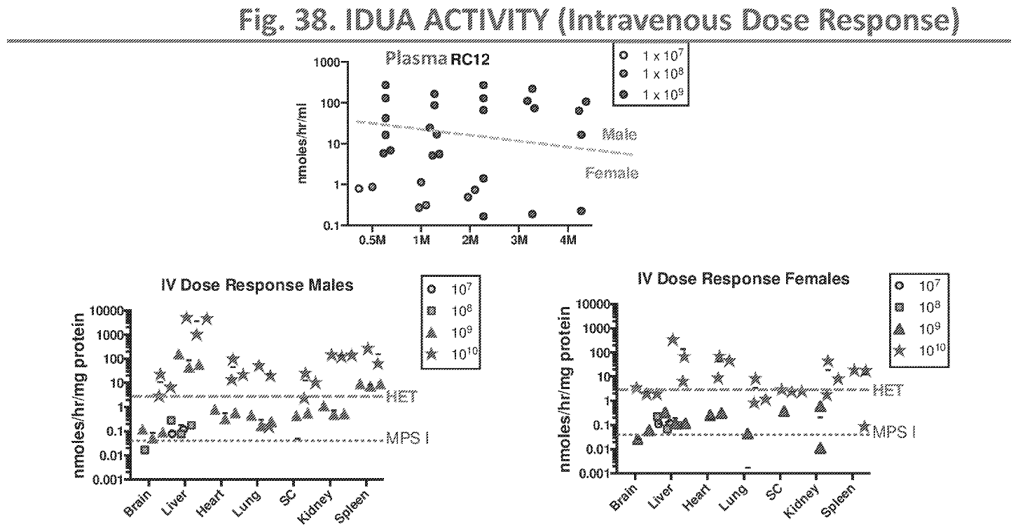

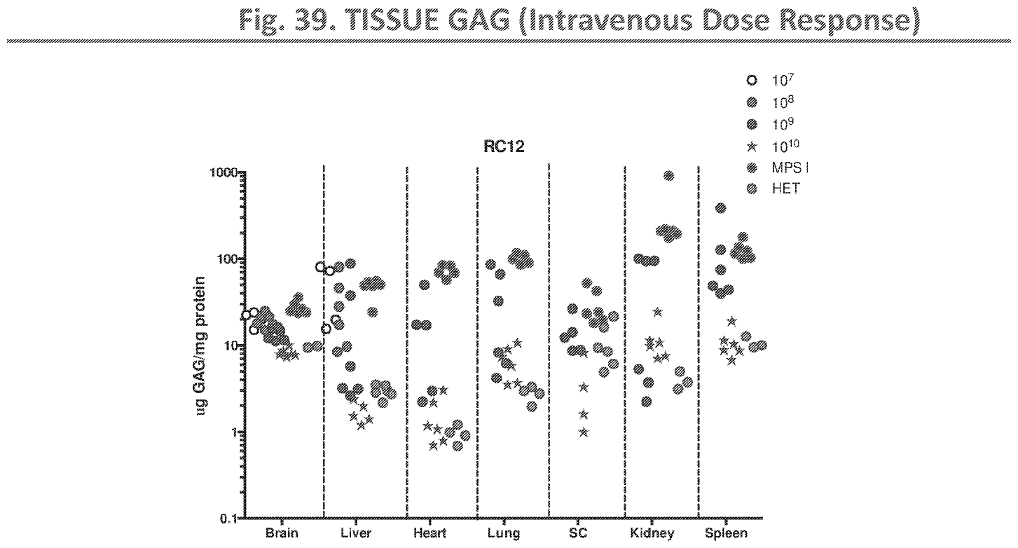

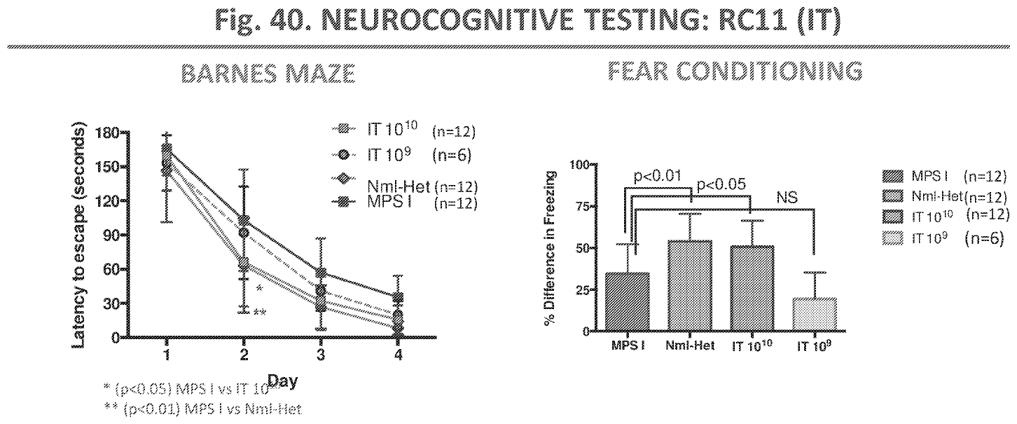

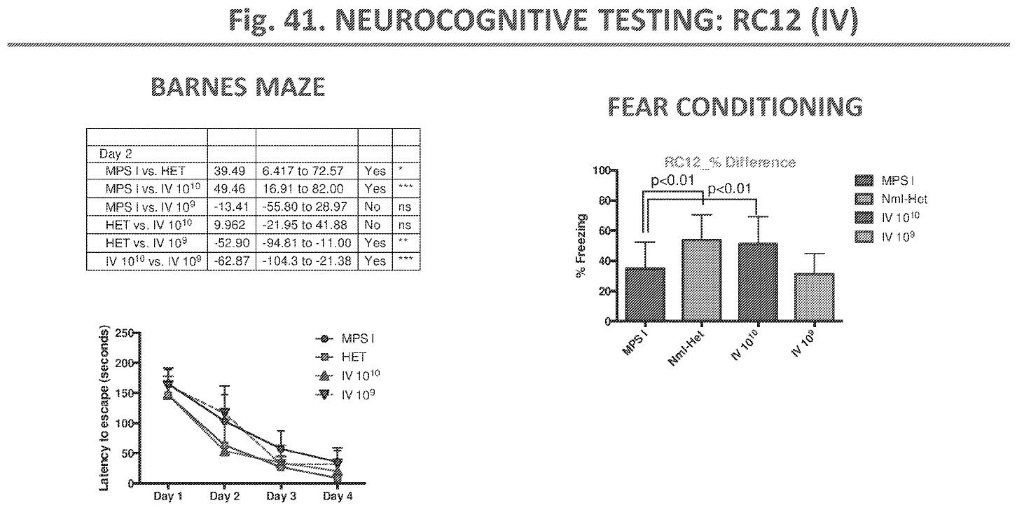

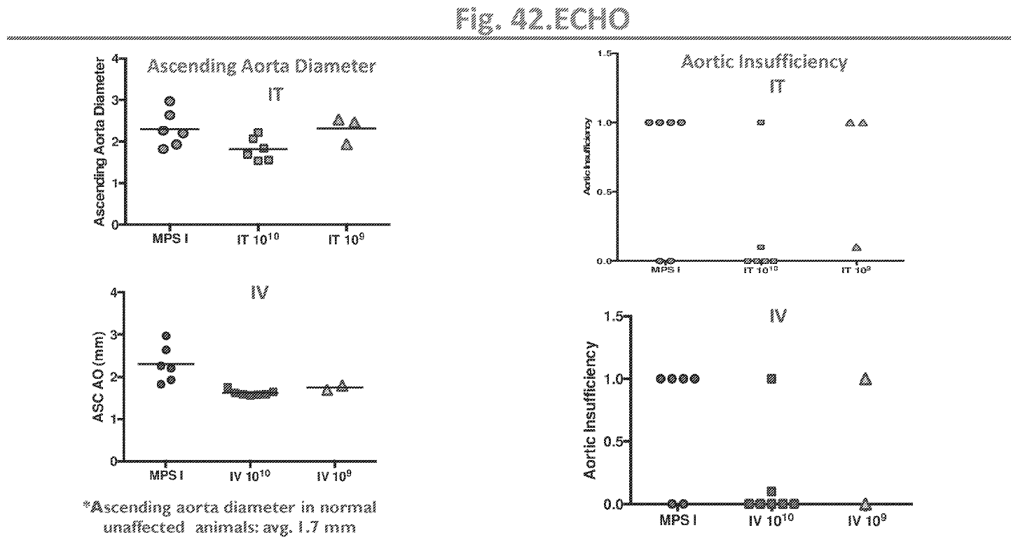

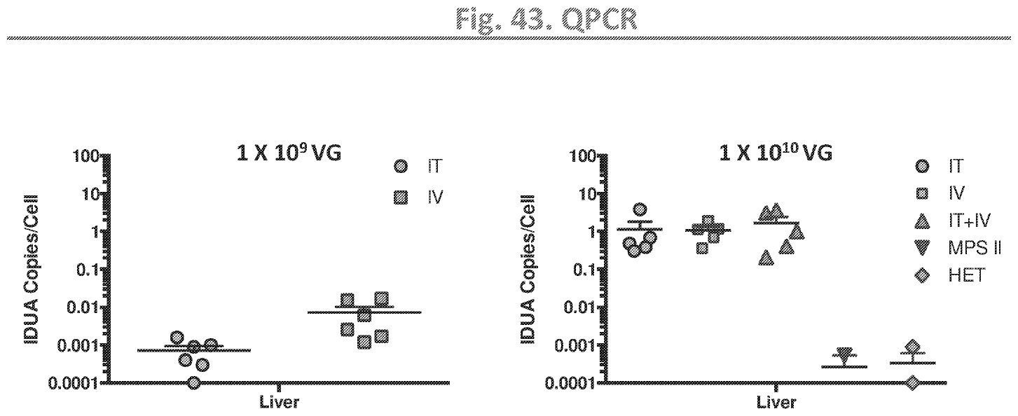

The data (FIGS. 24-43) showed that mice treated with RGX-111 gene therapy had high and sustained levels of IDUA enzyme activity in plasma and tissues regardless of route of administration. Normalization of GAG levels were observed in all tissues studied. Prevention of neurocognitive decline was observed in treated animals. Prevention of aortic dysfunction was noted in treated mice. Male mice treated with AAV9-IDUA treatment had normalization of skull width, zygomatic arch, and prevention of kyphosis.

Minimal Effective Dose (10⁷-10⁹ VG)

| | | | | | |
|---|---|---|---|---|---|
| Cohort | | | | | |
| RC11 | Treatment | DOB | Gender | Volume, Dose | Treatment Date |

| | | | | |
|---|---|---|---|---|
| 5978 | IT | M | 10 ul, 1 × 107 | Jul. 29, 2020 |
| 6003 | IT | M | 10 ul, 1 × 107 | Jul. 29, 2020 |
| 5981 | IT | F | 10 ul, 1 × 107 | Jul. 29, 2020 |
| 5982 | IT | F | 10 ul, 1 × 107 | Jul. 29, 2020 |
| 6008 | IT | M | 10 ul, 1 × 108 | Jul. 29, 2020 |
| 6010 | IT | M | 10 ul, 1 × 108 | Jul. 29, 2020 |
| 6017 | IT | M | 10 ul, 1 × 108 | Jul. 29, 2020 |
| 6001 | IT | F | 10 ul, 1 × 108 | Jul. 29, 2020 |
| 6013 | IT | F | 10 ul, 1 × 108 | Jul. 29, 2020 |
| 6022 | IT | F | 10 ul, 1 × 108 | Jul. 29, 2020 |
| 6018 | IT | M | 10 ul, 1 × 109 | Jul. 29, 2020 |
| 6019 | IT | M | 10 ul, 1 × 109 | Jul. 29, 2020 |
| 6020 | IT | M | 10 ul, 1 × 109 | Jul. 29, 2020 |
| 6026 | IT | F | 10 ul, 1 × 109 | Jul. 29, 2020 |
| 6028 | IT | F | 10 ul, 1 × 109 | Jul. 29, 2020 |
| 6029 | IT | F | 10 ul, 1 × 109 | Jul. 29, 2020 |

Mice treated with IT administered 10⁷-10⁹ vg RGX-111 had no detectable levels of IDUA enzyme activity in plasma and tissues, with no normalization of GAG levels in tissues.

Mice treated with IV administered 10⁷ and 10⁸ vg RGX-111 had no detectable levels of IDUA enzyme activity in plasma and tissues, with no normalization of GAG levels in tissues. However, there was enzyme activity at 10⁹ vg, with partial normalization of GAG levels at this dose. There was no prevention of neurocognitive decline in IT or IV treated animals. Cardiac assessments showed no improvement with the IT dose response, and were inconclusive with the IV dose response. These results characterize a minimal effective dose of 10¹⁰ vg for both IT and IV routes of administration.

### Example K

Higher and Lower Dose-Ranging Study Measuring Plasma IDS, Urine GAG, Tissue IDS, Tissue GAG, gPCR, Skeletal Analysis, and Behavior

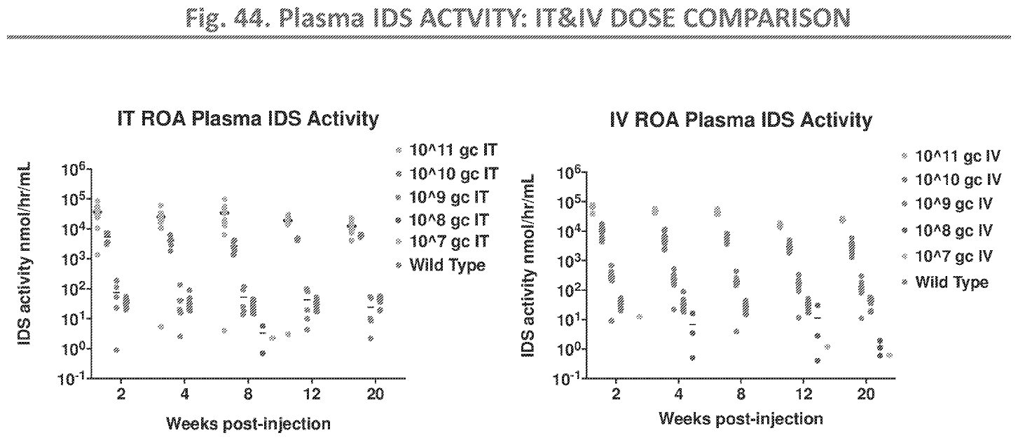

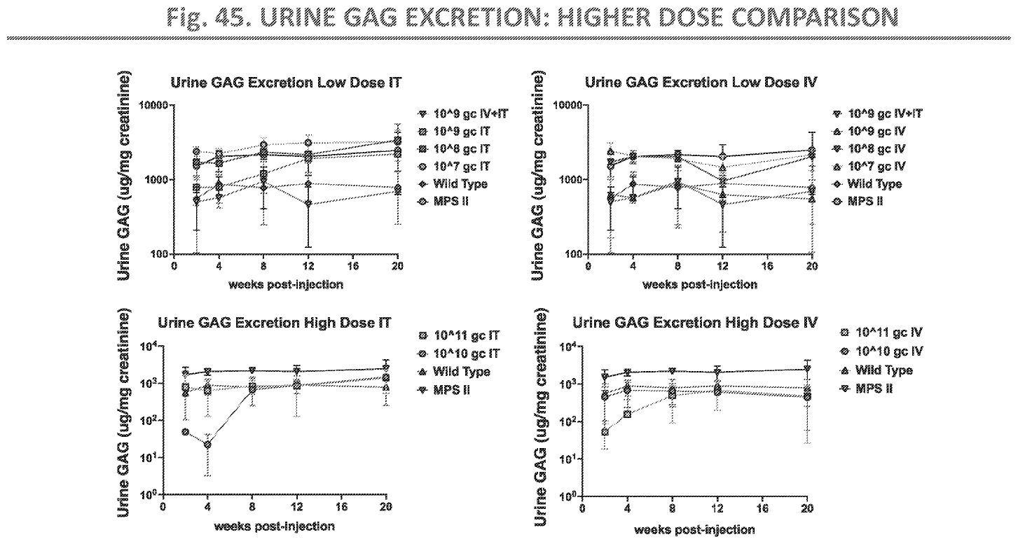

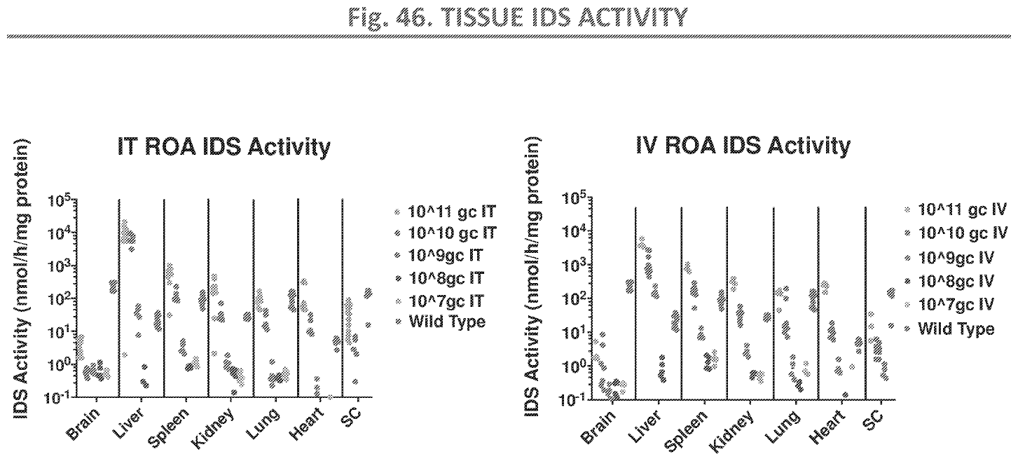

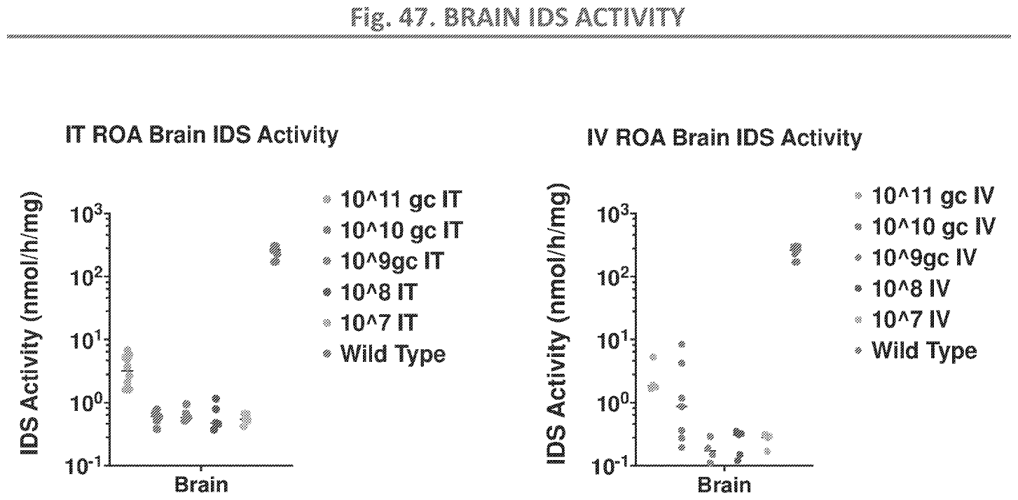

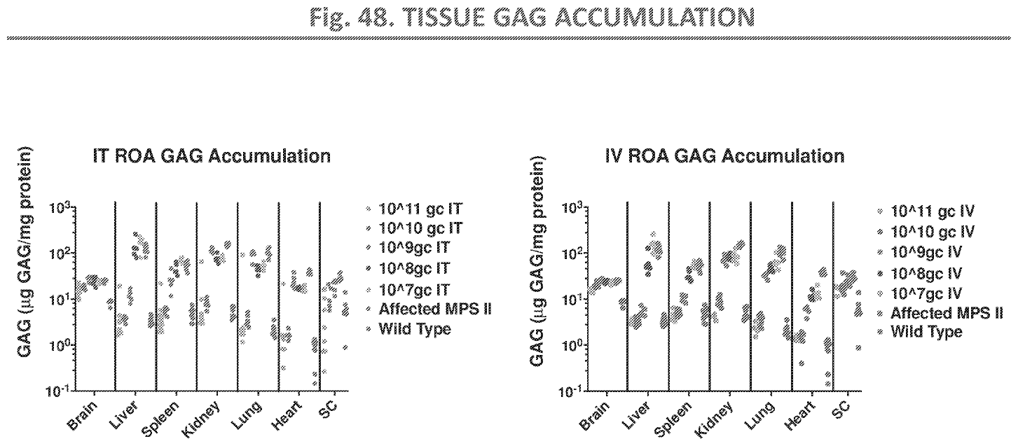

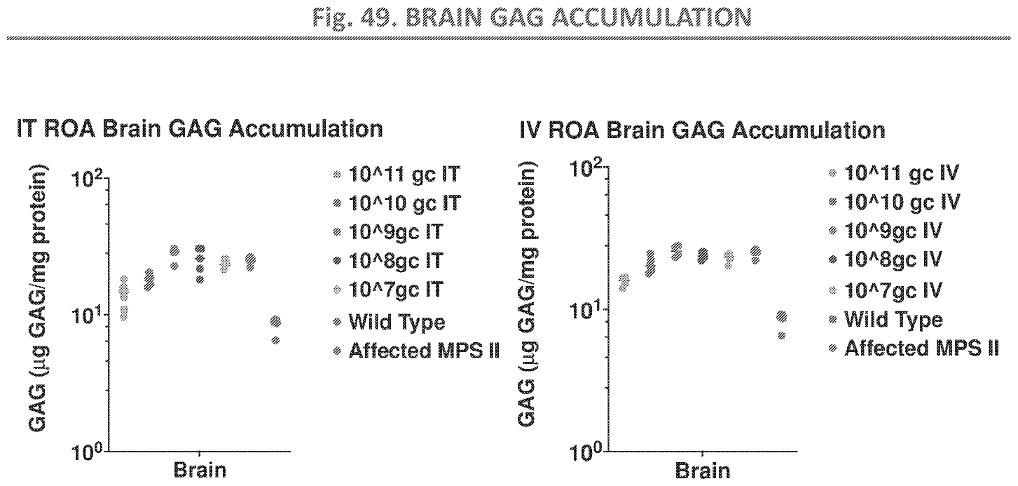

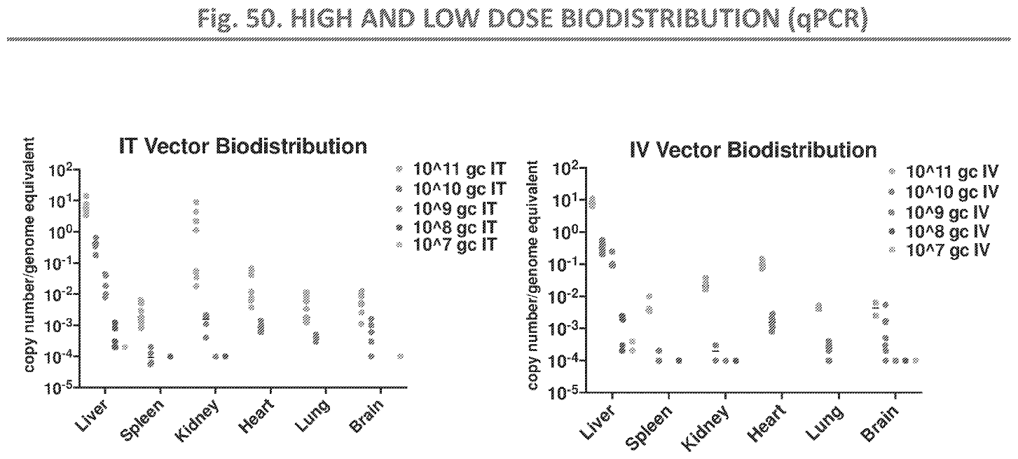

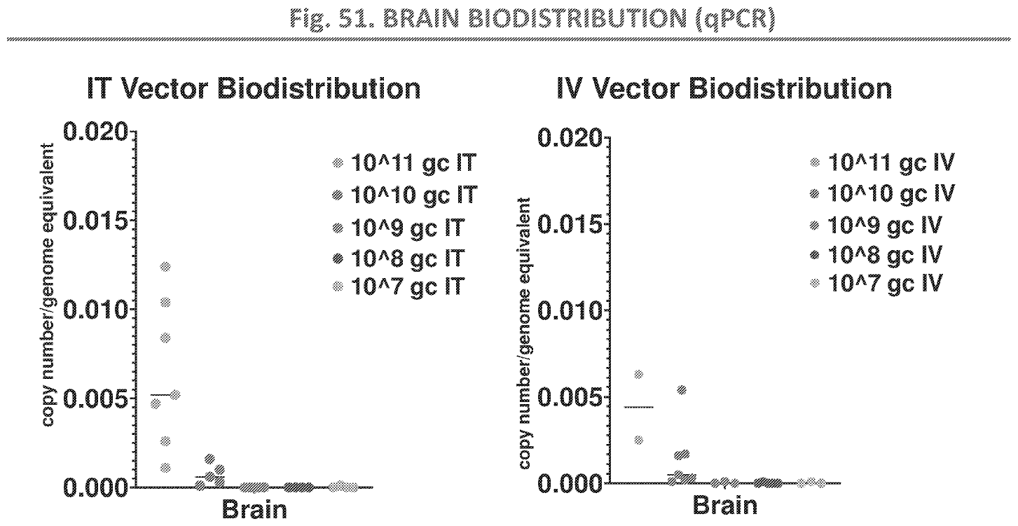

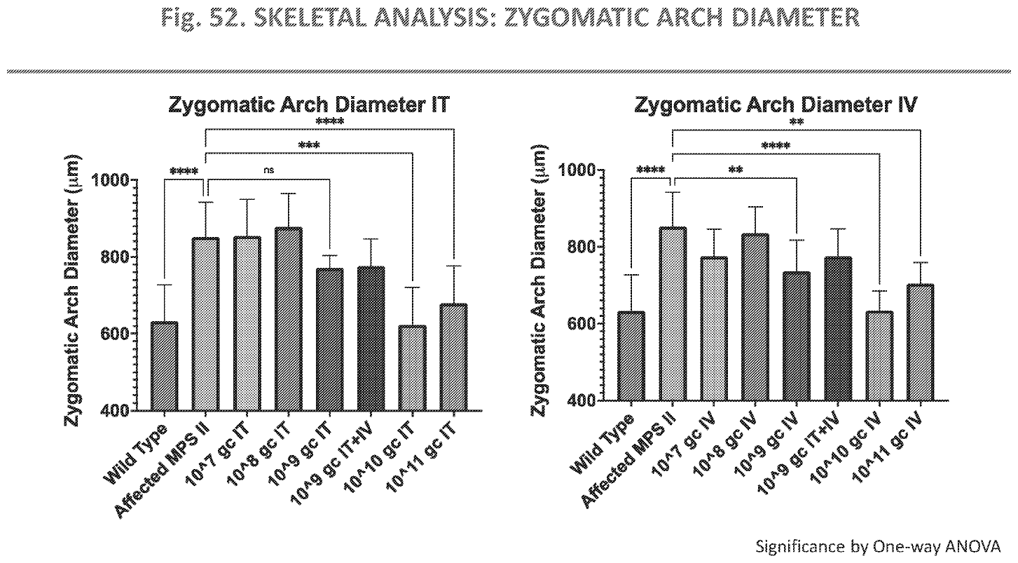

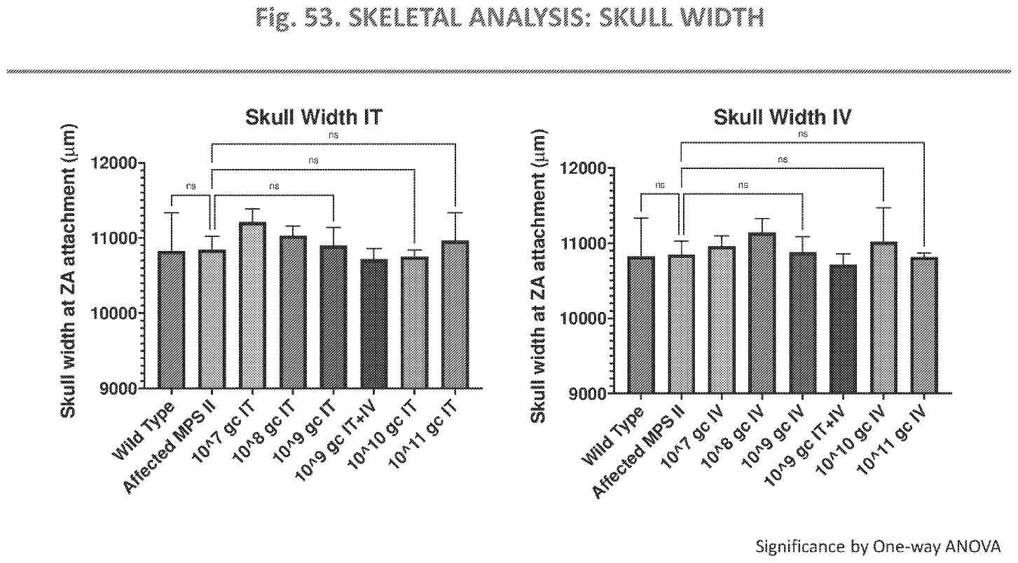

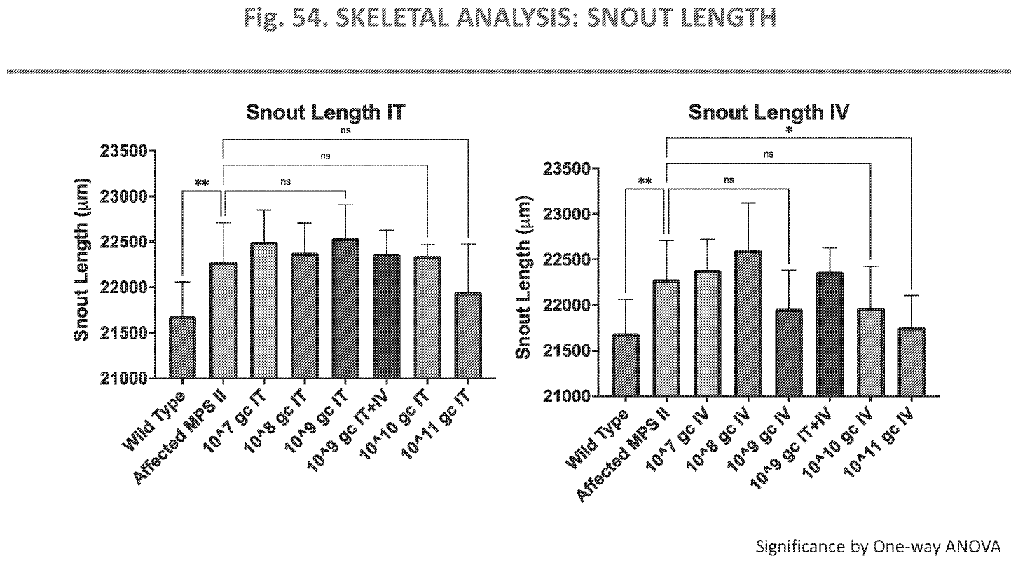

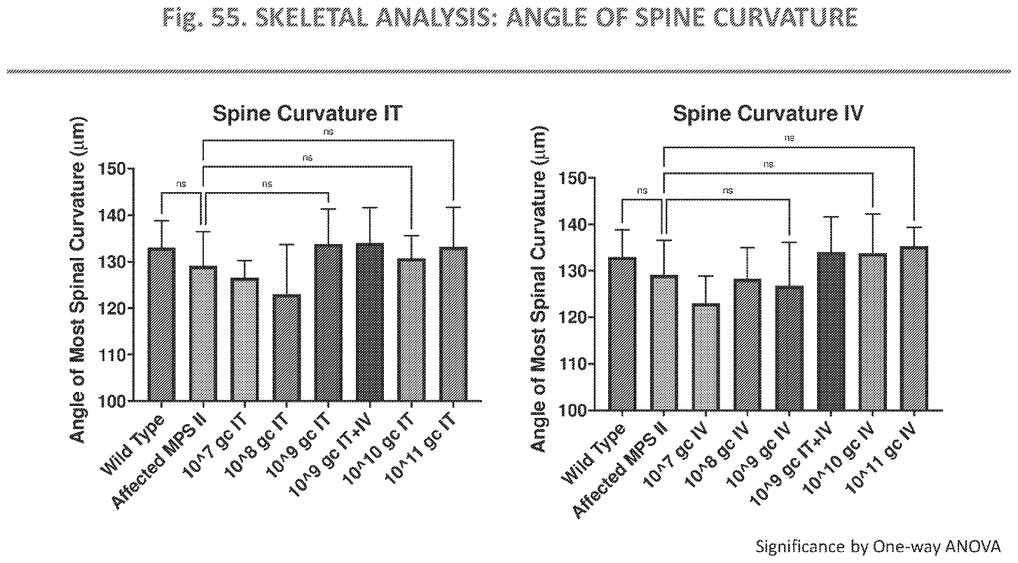

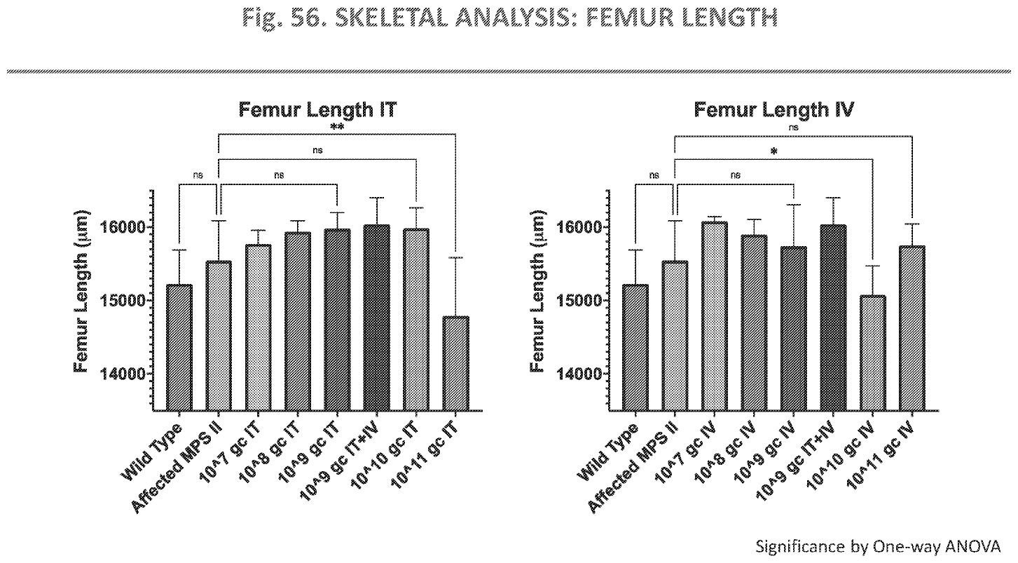

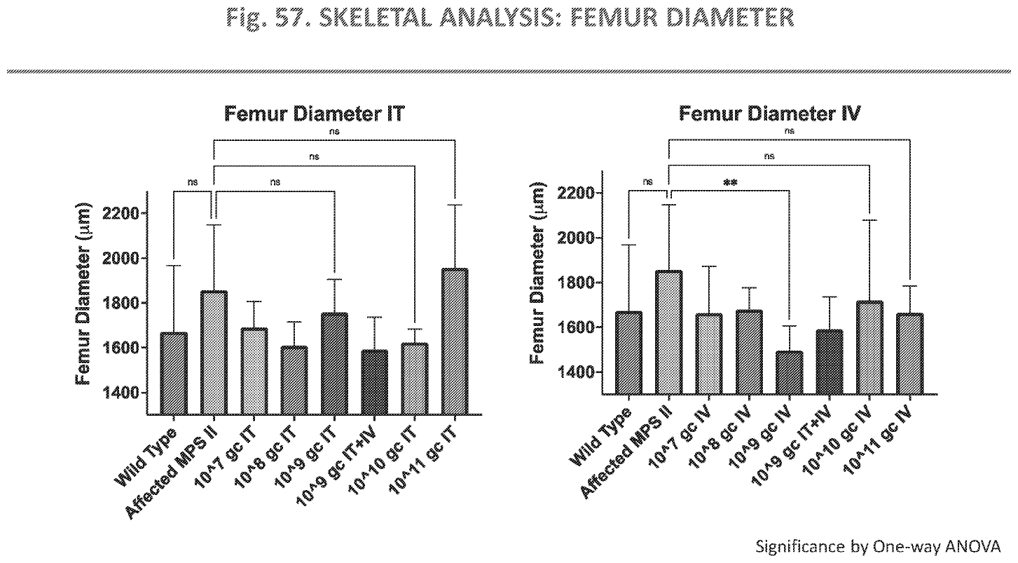

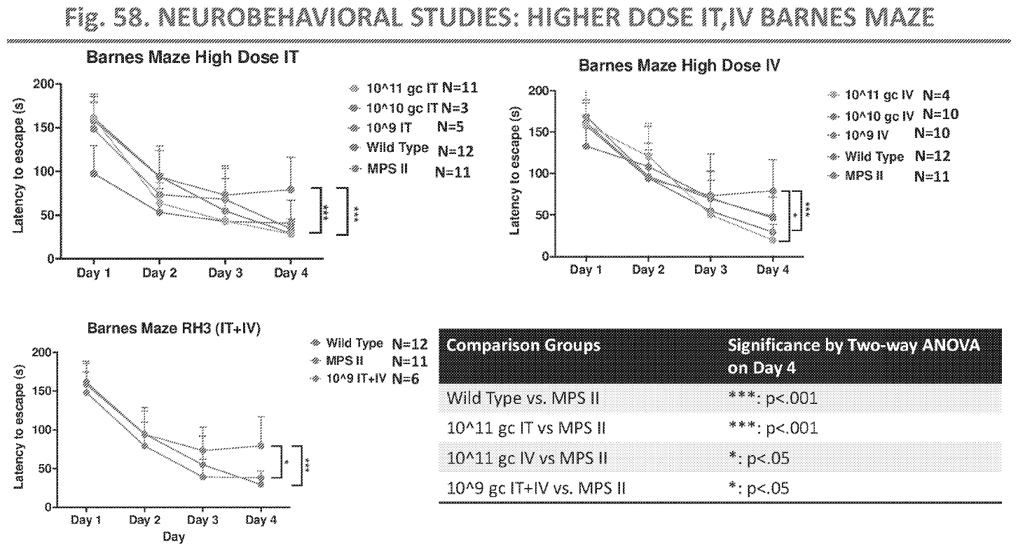

### See FIGS. 44-58.

| | MPSII AAV9 IDS | | | | | | | |
|---|---|---|---|---|---|---|---|---|
| IDS | IV ROA | | | | | | | |
| Animal | | Cohort | | | | | Injection | |
| # | Cage # | # | Dose | Lot # | DOB | Gender | Date | Euthanasia |

| | | | | | |
|---|---|---|---|---|---|
| 5678 | 1382 | RH2 | IV (10\hat{ }7 gc) | 121-191114-1 | M |
| 5679 | 1382 | RH2 | IV (10\hat{ }7 gc) | 121-191114-1 | M |
| 5680 | 1382 | RH2 | IV (10\hat{ }7 gc) | 121-191114-7 | M |
| 5681 | 1382 | RH2 | IV (10\hat{ }7 gc) | 121-191114-1 | M |
| 5687 | 1384 | RH2 | IV (10\hat{ }7 gc) | 121-191114-2-B | M |
| 5692 | 1386 | RH2 | IV (10\hat{ }8 gc) | 121-191114-7 | M |
| 5693 | 1386 | RH2 | IV (10\hat{ }8 gc) | 121-191114-2-B | M |
| 5694 | 1386 | RH2 | IV (10\hat{ }8 gc) | 121-191114-7 | M |
| 5695 | 1386 | RH2 | IV (10\hat{ }8 gc) | 121-191114-1 | M |
| 5700 | 1388 | RH2 | IV (10\hat{ }8 gc) | 121-191114-2-B | M |
| 5701 | 1388 | RH2 | IV (10\hat{ }9 gc) | 121-191114-7 | M |
| 5702 | 1388 | RH2 | IV (10\hat{ }9 gc) | 121-191114-2-B | M |
| 5703 | 1388 | RH2 | IV (10\hat{ }9 gc) | 121-191114-1 | M |
| 5709 | 1392 | RH2 | IV (10\hat{ }9 gc) | 121-191114-7 | M |
| 6088 | 1605 | RH4 | IV (10\hat{ }9 gc) | 121-191114-2-B | M |
| 6090 | 1605 | RH4 | IV (10\hat{ }9 gc) | 121-191114-2-B | M |
| 6091 | 1605 | RH4 | IV (10\hat{ }9 gc) | 121-191114-2-B | M |
| 6222 | 1642 | RH4 | IV (10\hat{ }9 gc) | 121-191114-2-B | M |
| 6223 | 1642 | RH4 | IV (10\hat{ }9 gc) | 121-191114-2-B | M |
| 6225 | 1642 | RH4 | IV (10\hat{ }9 gc) | 121-191114-2-B | M |
| 6690 | 1783 | RH6 | IV 10\hat{ }10 gc | 121-191114-3-B | M |
| 6691 | 1783 | RH6 | IV 10\hat{ }10 gc | 121-191114-3-B | M |
| 6696 | 1783 | RH6 | IV 10\hat{ }10 gc | 121-191114-3-B | M |
| 6683 | 1808 | RH6 | IV 10\hat{ }11 gc | 121-191114-4-B | M |
| 6687 | 1808 | RH6 | IV 10\hat{ }11 gc | 121-191114-4-B | M |
| 6695 | 1808 | RH6 | IV 10\hat{ }11 gc | 121-191114-4-B | M |
| 6099 | 1825 | RH6 | IV 10\hat{ }11 gc | 121-191114-4-B | M |
| 6703 | 1825 | RH6 | IV 10\hat{ }10 gc | 121-191114-3-B | M |
| 6705 | 1825 | RH6 | IV 10\hat{ }10 gc | 121-191114-3-B | M |
| 6892 | 1940 | RH7 | IV 10\hat{ }10 gc | 121-191114-3-B | M |
| 6888 | 1941 | RH7 | IV 10\hat{ }10 gc | 121-191114-3-B | M |
| 6889 | 1941 | RH7 | IV 10\hat{ }10 gc | 121-191114-3-B | M |
| 6890 | 1941 | RH7 | IV 10\hat{ }10 gc | 121-191114-3-B | M |
| 6885 | 1966 | RH7 | IV 10\hat{ }10 gc | 121-191114-3-B | M |
| 6887 | 1966 | RH7 | IV 10\hat{ }10 gc | 121-191114-3-B | M |

| | MPSII AAV9 IDS | | | | | | | |
|---|---|---|---|---|---|---|---|---|
| IDS | IV ROA | | | | | | | |
| Animal | | Cohort | | | | | Injection | |
| # | Cage # | # | Dose | Lot # | DOB | Gender | Date | Euthanasia |

| | | | | | |
|---|---|---|---|---|---|
| 5678 | 1382 | RH2 | IV (10\hat{ }7 gc) | 121-191114-1 | M |
| 5679 | 1382 | RH2 | IV (10\hat{ }7 gc) | 121-191114-1 | M |
| 5680 | 1382 | RH2 | IV (10\hat{ }7 gc) | 121-191114-7 | M |
| 5681 | 1382 | RH2 | IV (10\hat{ }7 gc) | 121-191114-1 | M |
| 5687 | 1384 | RH2 | IV (10\hat{ }7 gc) | 121-191114-2-B | M |
| 5692 | 1386 | RH2 | IV (10\hat{ }8 gc) | 121-191114-7 | M |
| 5693 | 1386 | RH2 | IV (10\hat{ }8 gc) | 121-191114-2-B | M |
| 5694 | 1386 | RH2 | IV (10\hat{ }8 gc) | 121-191114-7 | M |
| 5695 | 1386 | RH2 | IV (10\hat{ }8 gc) | 121-191114-1 | M |
| 5700 | 1388 | RH2 | IV (10\hat{ }8 gc) | 121-191114-2-B | M |
| 5701 | 1388 | RH2 | IV (10\hat{ }9 gc) | 121-191114-7 | M |
| 5702 | 1388 | RH2 | IV (10\hat{ }9 gc) | 121-191114-2-B | M |
| 5703 | 1388 | RH2 | IV (10\hat{ }9 gc) | 121-191114-1 | M |
| 5709 | 1392 | RH2 | IV (10\hat{ }9 gc) | 121-191114-7 | M |
| 6088 | 1605 | RH4 | IV (10\hat{ }9 gc) | 121-191114-2-B | M |
| 6090 | 1605 | RH4 | IV (10\hat{ }9 gc) | 121-191114-2-B | M |
| 6091 | 1605 | RH4 | IV (10\hat{ }9 gc) | 121-191114-2-B | M |
| 6222 | 1642 | RH4 | IV (10\hat{ }9 gc) | 121-191114-2-B | M |
| 6223 | 1642 | RH4 | IV (10\hat{ }9 gc) | 121-191114-2-B | M |
| 6225 | 1642 | RH4 | IV (10\hat{ }9 gc) | 121-191114-2-B | M |
| 6690 | 1783 | RH6 | IV 10\hat{ }10 gc | 121-191114-3-B | M |
| 6691 | 1783 | RH6 | IV 10\hat{ }10 gc | 121-191114-3-B | M |
| 6696 | 1783 | RH6 | IV 10\hat{ }10 gc | 121-191114-3-B | M |
| 6683 | 1808 | RH6 | IV 10\hat{ }11 gc | 121-191114-4-B | M |
| 6687 | 1808 | RH6 | IV 10\hat{ }11 gc | 121-191114-4-B | M |
| 6695 | 1808 | RH6 | IV 10\hat{ }11 gc | 121-191114-4-B | M |
| 6099 | 1825 | RH6 | IV 10\hat{ }11 gc | 121-191114-4-B | M |
| 6703 | 1825 | RH6 | IV 10\hat{ }10 gc | 121-191114-3-B | M |
| 6705 | 1825 | RH6 | IV 10\hat{ }10 gc | 121-191114-3-B | M |
| 6892 | 1940 | RH7 | IV 10\hat{ }10 gc | 121-191114-3-B | M |
| 6888 | 1941 | RH7 | IV 10\hat{ }10 gc | 121-191114-3-B | M |
| 6889 | 1941 | RH7 | IV 10\hat{ }10 gc | 121-191114-3-B | M |
| 6890 | 1941 | RH7 | IV 10\hat{ }10 gc | 121-191114-3-B | M |
| 6885 | 1966 | RH7 | IV 10\hat{ }10 gc | 121-191114-3-B | M |
| 6887 | 1966 | RH7 | IV 10\hat{ }10 gc | 121-191114-3-B | M |

| MPSII | MPSII Untreated | | | | | | Injection | |
|---|---|---|---|---|---|---|---|---|
| Animal # | Cage # | Cohort # | Dose | Lot # | DOB | Gender | Date | Euthanasia |

| | | | | | | |
|---|---|---|---|---|---|---|
| 5848 | 1498 | RH3 | MPS II | N/A | M | NA |
| | | | control | | | |
| 5849 | 1498 | RH3 | MPS II | N/A | M | NA |
| | | | control | | | |
| 5858 | 1507 | RH3 | MPS II | N/A | M | NA |
| | | | control | | | |
| 5860 | 1507 | RH3 | MPS II | N/A | M | NA |
| | | | control | | | |
| 5871 | 1519 | RH3 | MPS II | N/A | M | NA |
| | | | control | | | |
| 6227 | 1608 | RH4 | MPS II | N/A | M | NA |
| | | | control | | | |
| 6228 | 1608 | RH4 | MPS II | N/A | M | NA |
| | | | control | | | |
| 7008 | 2052 | RH9 | MPS II | N/A | M | NA |
| 7010 | 2052 | RH9 | MPS II | N/A | M | NA |
| 7011 | 2052 | RH9 | MPS II | N/A | M | NA |
| 7012 | 2052 | RH9 | MPS II | N/A | M | NA |

| Wt | Wild-type | | | | | | Injection | |
|---|---|---|---|---|---|---|---|---|
| Animal # | Cage # | Cohort # | Dose | Lot # | DOB | Gender | Date | Euthanasia |

| | | | | | | |
|---|---|---|---|---|---|---|
| 5847 | 1490 | RH3 | Wild | N/A | M | NA |
| | | | Type | | | |
| | | | Control | | | |
| 5859 | 1507 | RH3 | Wild | N/A | M | NA |
| | | | Type | | | |
| | | | Control | | | |
| 6089 | 1605 | RH4 | Wild | N/A | M | NA |
| | | | Type | | | |
| | | | control | | | |
| 6226 | 1608 | RH4 | Wild | N/A | M | NA |
| | | | Type | | | |
| | | | control | | | |
| 6230 | 1608 | RH4 | Wild | N/A | M | NA |
| | | | Type | | | |
| | | | control | | | |
| 6224 | 1642 | RH4 | Wild | N/A | M | NA |
| | | | Type | | | |
| | | | control | | | |
| 6689 | 1783 | RH6 | Wild | N/A | M | NA |
| | | | Type | | | |
| | | | control | | | |
| 6704 | 1825 | RH6 | Wild | N/A | M | NA |
| | | | Type | | | |
| | | | control | | | |
| 6891 | 1940 | RH7 | Wild | N/A | M | NA |
| | | | Type | | | |
| | | | control | | | |
| 6893 | 1940 | RH7 | Wild | N/A | M | NA |
| | | | Type | | | |
| | | | control | | | |
| 7009 | 2053 | RH9 | Wild | N/A | M | NA |
| | | | Type | | | |
| | | | control | | | |
| 7013 | 2053 | RH9 | Wild | N/A | M | NA |
| | | | Type | | | |
| | | | control | | | |

| | MPSII AAV9 IDS | | | | | | | |
|---|---|---|---|---|---|---|---|---|
| IDS | IT + IV ROA | | | | | | Injection | |
| Animal # | Cage # | Cohort # | Dose | Lot # | DOB | Gender | Date | Euthanasia |

| | | | | | |
|---|---|---|---|---|---|
| 5846 | 1490 | RH3 | IT/IV | 121-19111-4- | M |
| | | | (both | 3-A (IT)/121- | |
| | | | 10\hat{ }9 gc) | 191114-2-B | |
| | | | | (IV) | |
| 5851 | 1499 | RH3 | IT/IV | 121-19111-4- | M |
| | | | (both | 3-A (IT)/121- | |
| | | | 10\hat{ }9 gc) | 191114-2-B | |
| | | | | (IV) | |
| 5864 | 1518 | RH3 | IT/IV | 121-19111-4- | M |
| | | | (both | 3-A (IT)/121- | |
| | | | 10\hat{ }9 gc) | 191114-2-B | |
| | | | | (IV) | |
| 5866 | 1518 | RH3 | IT/IV | 121-19111-4- | M |
| | | | (both | 3-A (IT)/121- | |
| | | | 10\hat{ }9 gc) | 191114-2-B | |
| | | | | (IV) | |
| 5867 | 1518 | RH3 | IT/IV | 121-19111-4- | M |
| | | | (both | 3-A (IT)/121- | |
| | | | 10\hat{ }9 gc) | 191114-2-B | |
| | | | | (IV) | |
| 5868 | 1518 | RH3 | IT/IV | 121-19111-4- | M |
| | | | (both | 3-A (IT)/121- | |
| | | | 10\hat{ }9 gc) | 191114-2-B | |
| | | | | (IV) | |

Supraphysiologic levels of IDS were observed in plasma and tissues in MPS II mice administered RGX-121 at doses of 10¹⁰ vc IV or 10¹¹ vc IT. Lower doses were ineffective. Normalized GAG storage levels were observed in urine and tissues. Normalized Zygomatic Arch is evidence for improved skeletal outcomes. Reduced Brain GAG and prevention of neurologic deficit were observed in animals administered 10¹¹ vc/mouse IT.

### Example L

In contrast to the normal mice, note the extensive, focal to multifocal, intraneuronal vacuolization (black arrows) within cells of the olfactory bulb, thalamus, hippocampus, rostral cerebral cortex, pons, and cerebellar cortex in the untreated mice. Decreased neuronal vacuolization is observed in the intravenous (IV) and intrathecally (IT) treated mice with only rare cerebellar vacuolization observed in the combination (IT+IV) treated mice.

In contrast to the normal mice, note the extensive, multifocal, intraneuronal vacuolization (black arrows) within cells of the dorsal root ganglion and dorsal horn of the untreated mice. Decreased neuronal vacuolization is observed in the intravenous (IV) and intrathecally (IT) treated mice with only rare vacuolization observed in the ganglion neurons of the combination (IT+IV) treated mice.

In contrast to the normal mice, note the multifocal, intraneuronal vacuolization (black arrows) within neurons of the dorsal thalamic nucleus in the untreated mice. These vacuoles correspond with the accumulation of Alcian Blue-positive material. Intraneuronal vacuolization and Alcian Blue positive material is not observed in the any of the 3 treatment groups.

In contrast to the normal mice, note the multifocal, intraneuronal vacuolization (black arrows) within Purkinje cells of the cerebellar cortex in the untreated mice. Progressively decreasing numbers of intraneuronal vacuoles were identified in the Purkinje cells of the intravenous, IT, and IV+IT treated mice. In contrast, lesser amounts of Alcian Blue-positive material were seen only in the untreated mice. Alcian Blue positive material is not observed in the any of the 3 treatment groups.

In contrast to the normal mice, note the accumulation of vacuolated cells (black arrows) within the centrolobular region of the liver in untreated mice. These vacuoles correspond with the accumulation of Alcian Blue-positive material. Vacuolated cells and Alcian Blue positive material are not observed in the any of the 3 treatment groups.

In contrast to the control mice, in which there is extensive hematopoietic activity, there is loss of hematopoietic precursors in the spleen of the untreated mice. In the treated mice, there is a progressive increase in the density of myeloid, erythroid, and platelet precursors. In addition, note the marked vacuolization of the stromal and smooth muscle cells of the splenic capsule in the untreated mice.

In contrast to the control mice, there is extensive vacuolization of the tunica media of large extra-cardiac vessels with infiltration of inflammatory cells in the untreated mice. Similarly, modest numbers of inflammatory cells are identified surrounding the intracardiac vessels of the untreated mice. In the treated mice, vascular pathology is not identified in the IV-treated mice and there is decreased tunica media vacuolization and inflammatory infiltration in the IT and IV+IT treated mice.

### Example M

[symbol]

| MOUSE | | | MOUSE | | | | |
|---|---|---|---|---|---|---|---|
| #/COHORT | SEX | GROUP # | ID | Tx | Cerebellum | Liver | [symbol] |
| 5429 (RC7) | MALE | 1 | 1 | IT | 0 | 0 | [symbol] |
| 5607 (RC10) | MALE | 1 | 2 | IT | 2 | 2 | [symbol] |
| 5034 (RC4) | FEMALE | 1 | 3 | IT | 0 | 2 | [symbol] |
| 5328 (RC7) | FEMALE | 1 | 4 | IT | 1 | 2 | [symbol] |
| 5258 (RC5) | MALE | 2 | 5 | IV | 2 | 0 | [symbol] |
| 5340 (RC8) | MALE | 2 | 6 | IV | 2 | 0 | [symbol] |
| 5262 (RC5) | FEMALE | 2 | 7 | IV | 2 | 0 | [symbol] |
| 5443 (RC8) | FEMALE | 2 | 8 | IV | 2 | 0 | [symbol] |
| 5268 (RC6) | MALE | 3 | 9 | IT + IV | 0 | 0 | [symbol] |
| 5626 (RC10) | MALE | 3 | 10 | IT + IV | 0 | 0 | [symbol] |
| 5272 (RC6) | FEMALE | 3 | 11 | IT + IV | 1 | 0 | [symbol] |
| 5496 (RC9) | FEMALE | 3 | 12 | IT + IV | 0 | 0 | [symbol] |
| 5518 (RC9) | MALE | 4 | 13 | Normal | 0 | 0 | [symbol] |
| 5483 (RC9) | MALE | 4 | 14 | Normal | 0 | 0 | [symbol] |
| 5321 (RC7) | FEMALE | 4 | 15 | Normal | 1 | 0 | [symbol] |
| 5613 (RC10) | FEMALE | 4 | 16 | Normal | 0 | 0 | [symbol] |
| 5012 (RC4) | MALE | 5 | 17 | Untreated | 3 | 3 | [symbol] |
| 5432 (RC7) | MALE | 5 | 18 | Untreated | 3 | 2 | [symbol] |
| 5010 (RC4) | FEMALE | 5 | 19 | Untreated | 3 | 2 | [symbol] |
| 5631 (RC10) | FEMALE | 5 | 20 | Untreated | 3 | 3 | [symbol] |

[symbol]

indicates data missing or illegible when filed

[symbol]

| MOUSE | | | | | | |
|---|---|---|---|---|---|---|
| #/COHORT | SEX | GROUP # | MOUSE ID | Tx | Cerebellum | Liver |
| 5429 (RC7) | MALE | 1 | 1 | IT | 1 | 0 |
| 5607 (RC10) | MALE | 1 | 2 | IT | 2 | 3 |
| 5034 (RC4) | FEMALE | 1 | 3 | IT | 0 | 0 |
| 5328 (RC7) | FEMALE | 1 | 4 | IT | 1 | 0 |
| 5258 (RC5) | MALE | 2 | 5 | IV | 1 | 0 |
| 5340 (RC8) | MALE | 2 | 6 | IV | 1 | 0 |
| 5262 (RC5) | FEMALE | 2 | 7 | IV | 2 | 0 |
| 5443 (RC8) | FEMALE | 2 | 8 | IV | 2 | 0 |
| 5268 (RC6) | MALE | 3 | 9 | IT + IV | 0-1 | 0 |
| 5626 (RC10) | MALE | 3 | 10 | IT + IV | 0 | 0 |
| 5272 (RC6) | FEMALE | 3 | 11 | IT + IV | 0 | 0 |
| 5496 (RC9) | FEMALE | 3 | 12 | IT + IV | 0 | 0 |
| 5518 (RC9) | MALE | 4 | 13 | Normal | 0 | 0 |
| 5483 (RC9) | MALE | 4 | 14 | Normal | 0 | 0 |
| 5321 (RC7) | FEMALE | 4 | 15 | Normal | 0 | 0 |
| 5613 (RC10) | FEMALE | 4 | 16 | Normal | 0 | 0 |
| 5012 (RC4) | MALE | 5 | 17 | Untreated | 3 | 3 |
| 5432 (RC7) | MALE | 5 | 18 | Untreated | 3 | 3 |
| 5010 (RC4) | FEMALE | 5 | 19 | Untreated | 2 | 2 |
| 5631 (RC10) | FEMALE | 5 | 20 | Untreated | 3 | 3 |

### MPSII

In the olfactory bulb, there is persistent neuronal vacuolization in the IV mice, but slightly decreased vacuolization in the IT as compared to the untreated mice (MPSII).

In the olfactory cortex, there is decreased, but persistent neuronal vacuolization in the IV mice, but a near complete resolution of vacuolization in the IT mice.

In the cerebral cortex, there is persistent neuronal vacuolization in the IV mice, but decreased neuronal vacuolization in the IT mice as compared to the untreated mice.

In the hippocampus, there is decreased neuronal vacuolization in the IV mice, and a near complete resolution of vacuolization in the IT mice as compared to the untreated mice.

In the hypothalamus, there is decreased, but persistent neuronal vacuolization in both IV mice, but near complete resolution in the IT mice as compared to the untreated mice (A-B)

In the thalamus, there is decreased, but persistent neuronal vacuolization in the IV mice, but a near complete resolution of vacuolization in the IT mice.

In the cerebellar purkinje cells, there is decreased (but persistent) neuronal vacuolization in both the IV and IT mice as compared to the untreated mice.

In the medulla, there is persistent neuronal vacuolization in the IV mice, but a near complete resolution of vacuolization in the IT mice.

As compared to the untreated mice, both groups of mice demonstrate decreased neuronal vacuolization, including anatomic distribution and severity. However, the IT-treated mice generally demonstrated more dramatic decreases in vacuolization (as compared to the IV-treated mice).

### Example O

Treatment of Cardiac, Neurologic, and Skeletal Manifestations of Murine MPS I with AAV9-IDUA: Efficacy Study of Vector Dose and Route of Administration

MucopolysaccharidosisType I (MPS I) is caused by absence of functional lysosomal enzyme alpha-L-iduronidase (IDUA) which catalyzes degradation of glycosaminoglycans (GAGs), MPS I is a multisystemic, chronic and progressive disease exhibiting growth delay, organomegaly, cardiopulmonary disease, skeletal dysplasia and neurocognitive decline. The most severe and prevalent form of MPS I is Hurler syndrome. Untreated patients normally do not survive beyond age 10.

Current treatments include enzyme replacement therapy (ERT). However, the enzyme does not cross the blood brain barrier and has limited effect on cardiac, neurologic, and skeletal manifestations Another current treatment is hematopoietic stem cell transplant (HSCT). However, HSCT results in low enzyme levels and there is a wide variability in neurologic effectiveness, often with below normal IQ and impaired neurocognitive ability.

To assess the most effective dose and route of administration of AAV9-IDUA vector on the cardiac, neurologic, and skeletal manifestations in MPS I mouse, the following groups were examined.

### Groups:

| Animal | Route of AAV Treatment | Dose of AAV9-IDUA | |
|---|---|---|---|
| MPS I | Intrathecal (IT) | 10⁷, 10⁸, 10⁹, 10¹⁰ | vg |
| MPS I | Intravenous (IV) | 10⁷, 10⁸, 10⁹, 10¹⁰ | vg |
| MPS I | Intrathecal + | 10¹⁰ | vg |
| | Intravenous (IT + IV) | | |

| Nml-Het | No treatment | No vector (normal control) |
|---|---|---|
| MPS I | No treatment | No vector (affected control) |

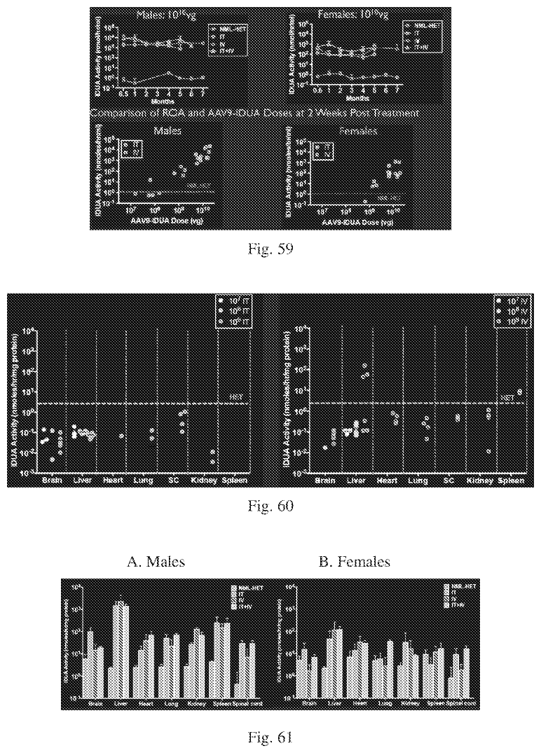

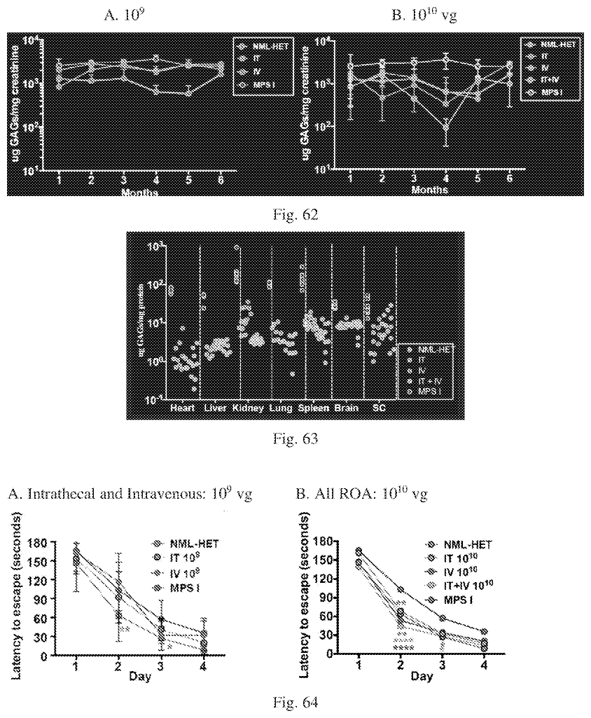

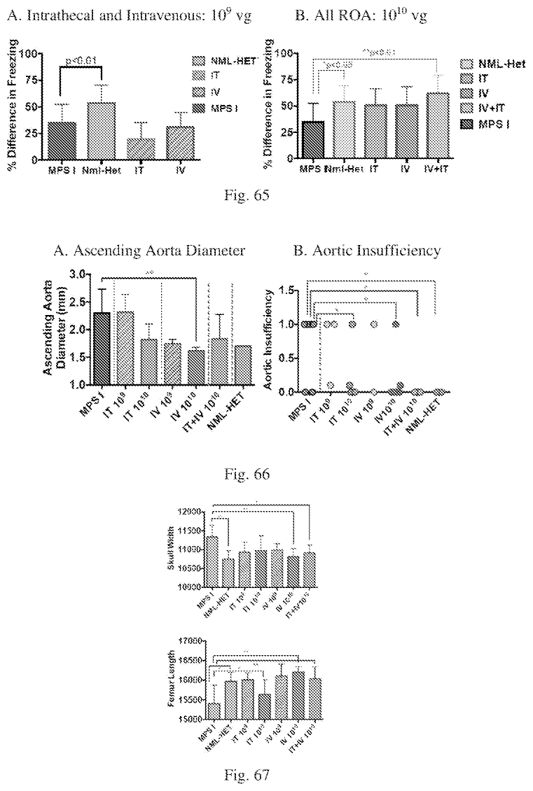

### See FIGS. 59-67.

### Example P

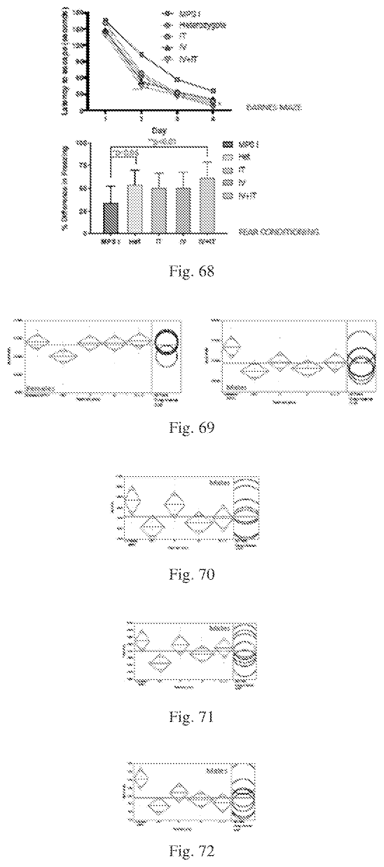

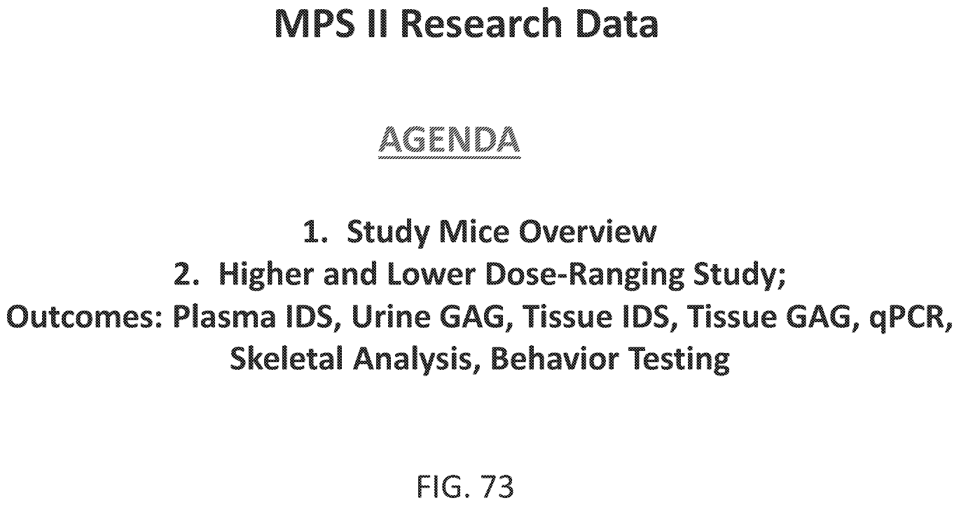

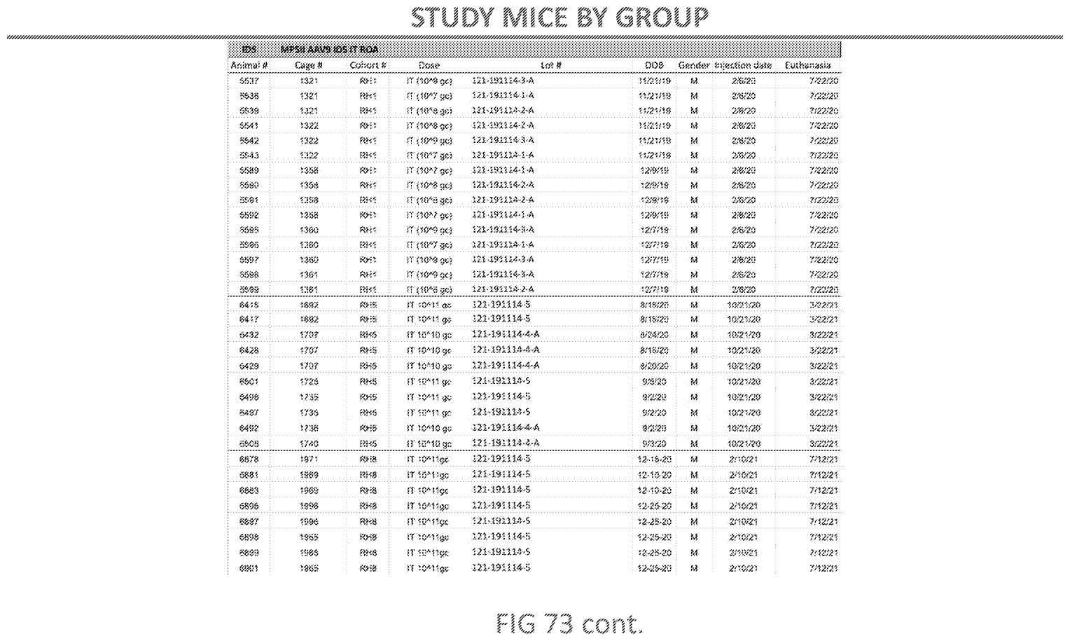

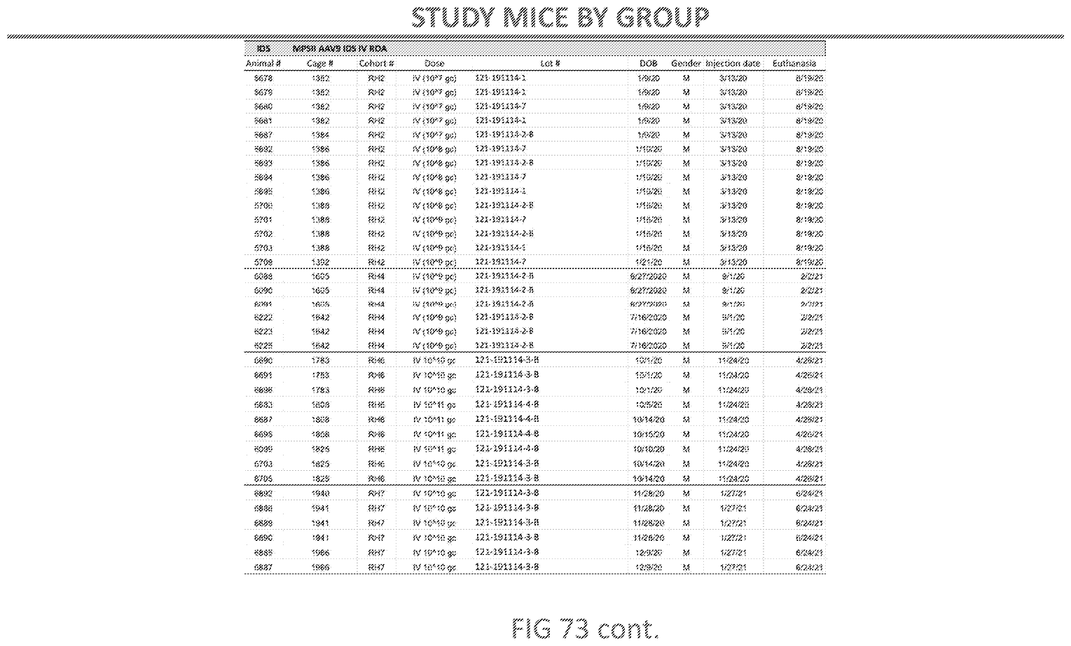

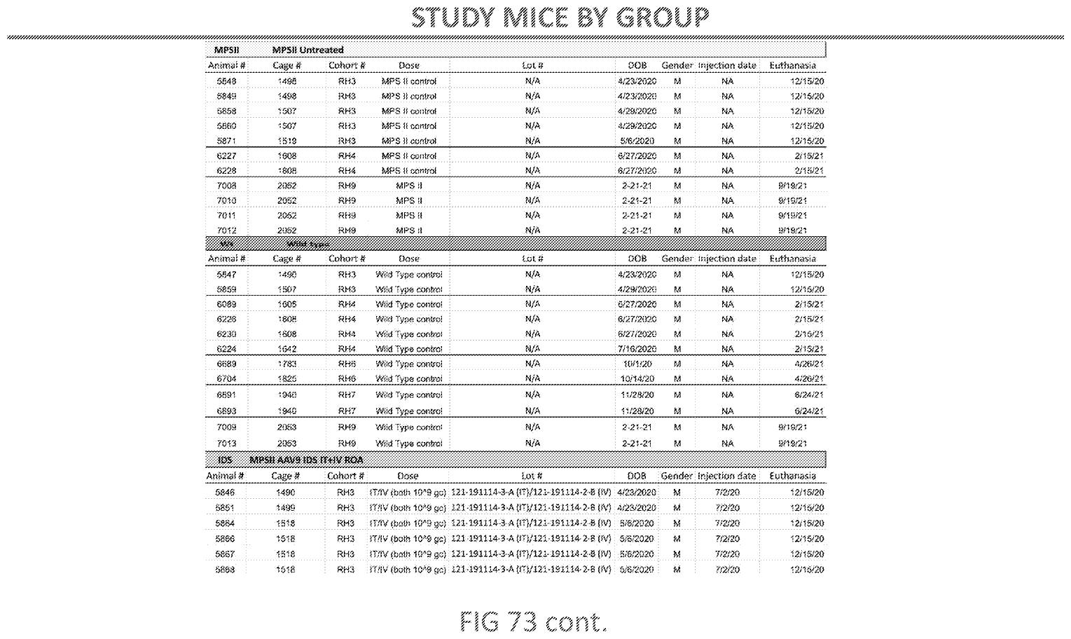

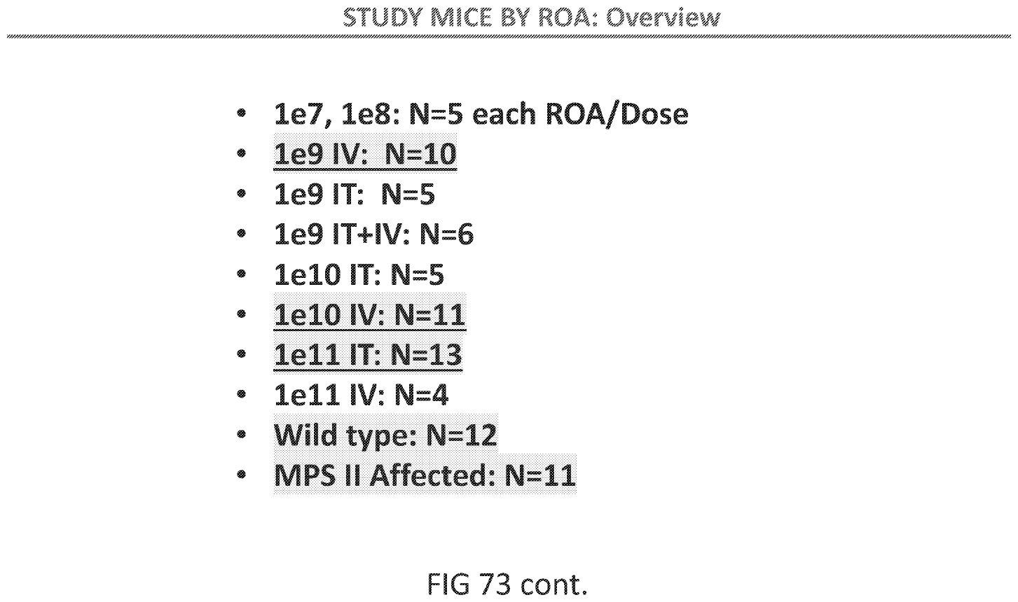

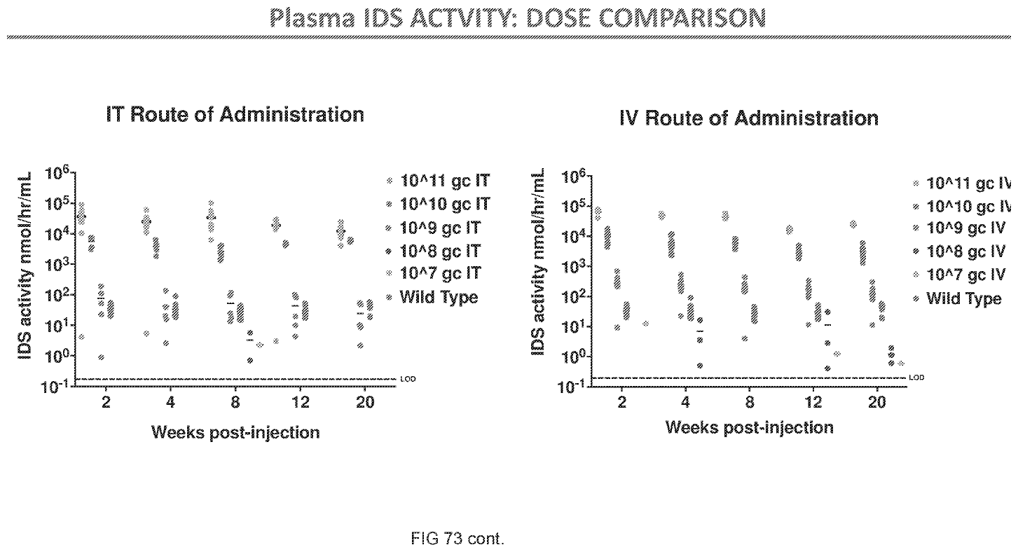

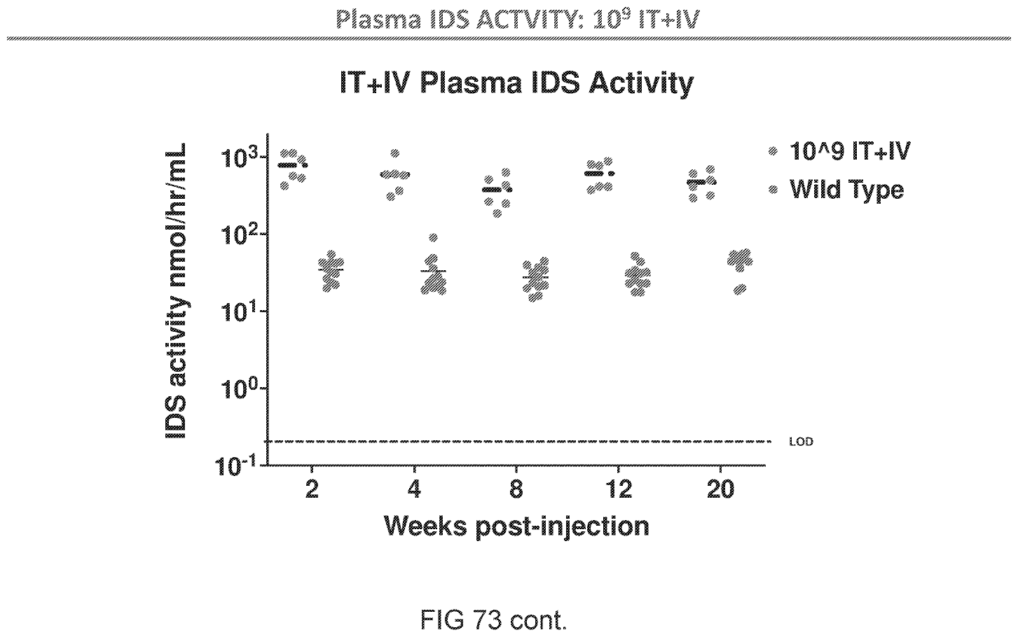

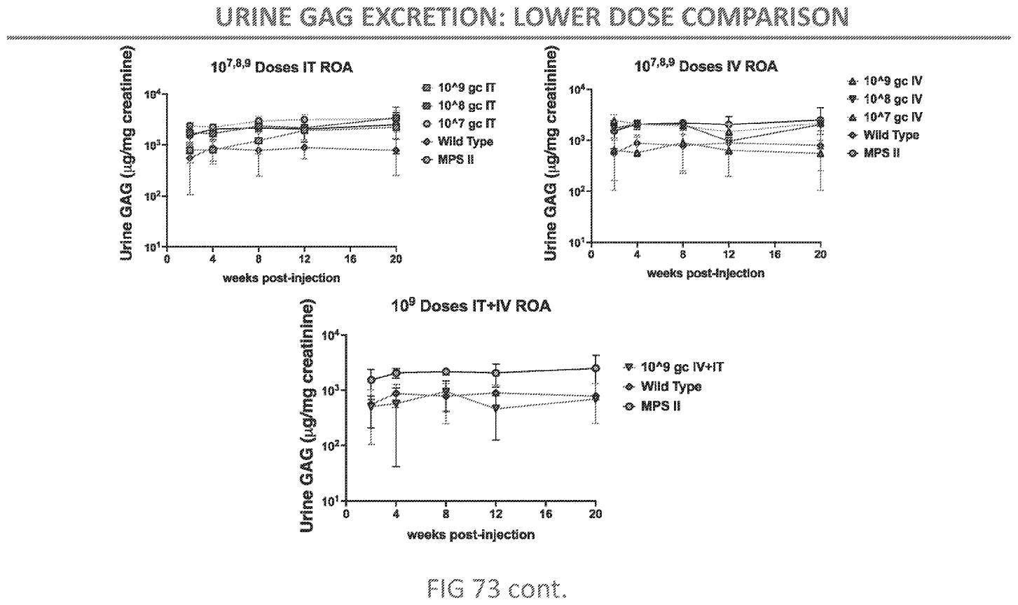

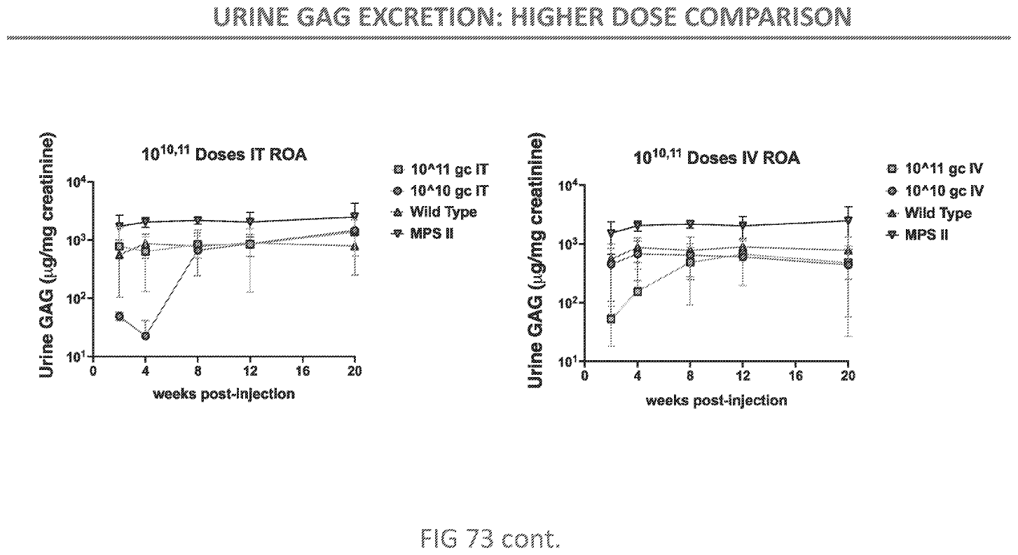

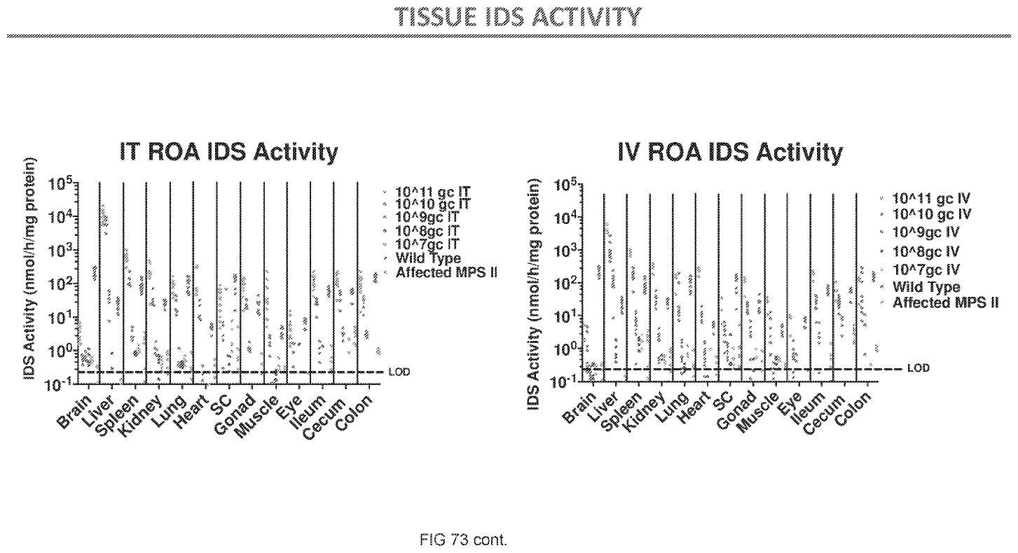

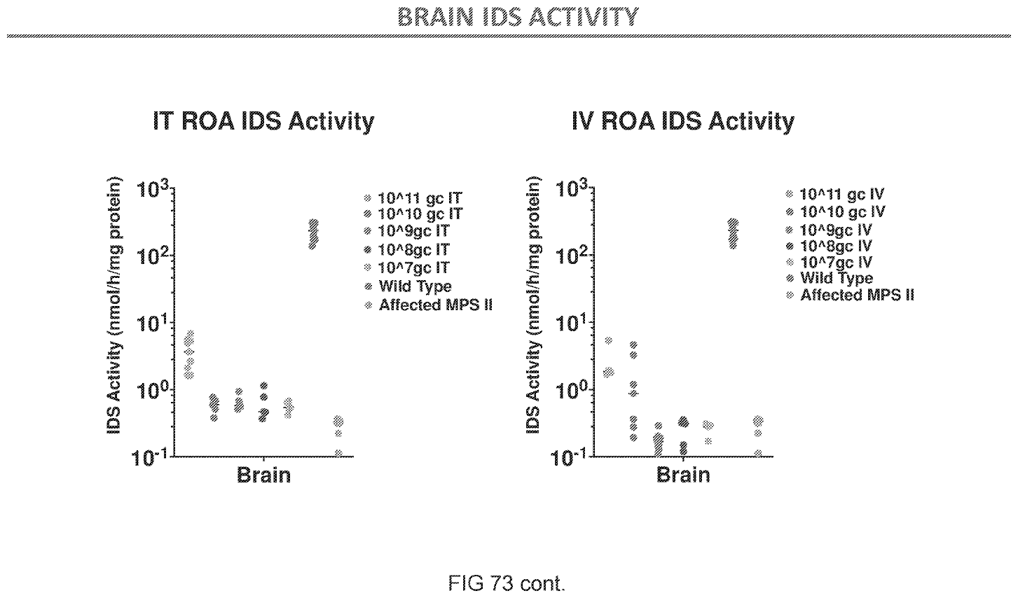

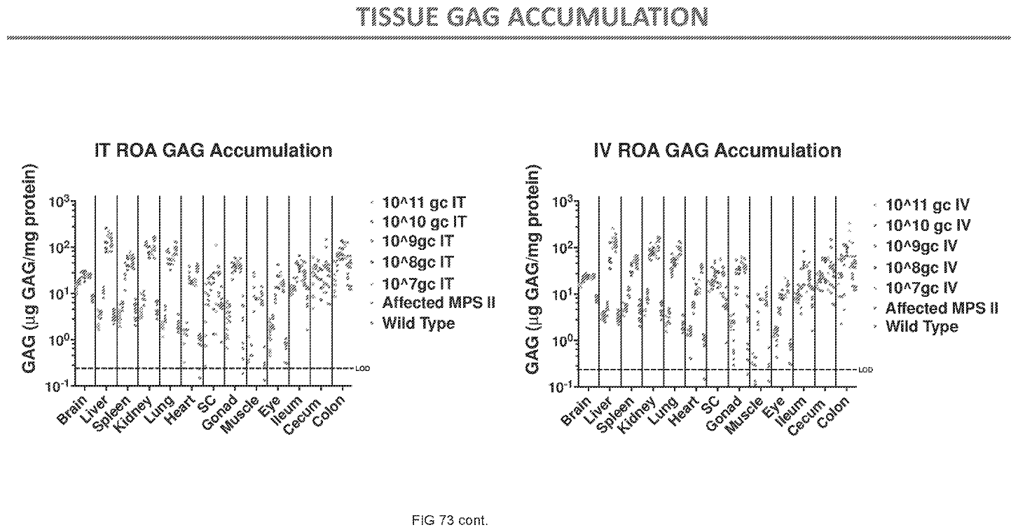

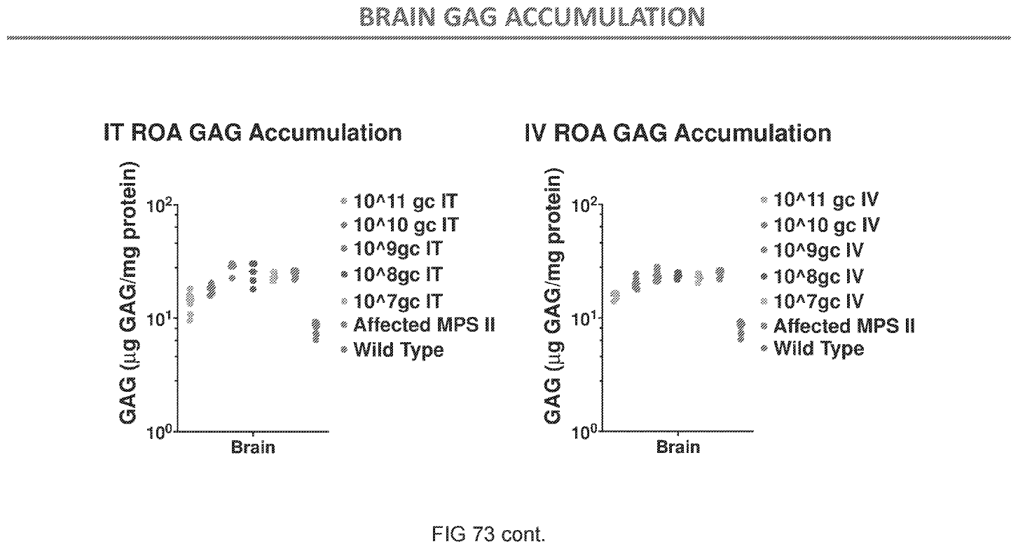

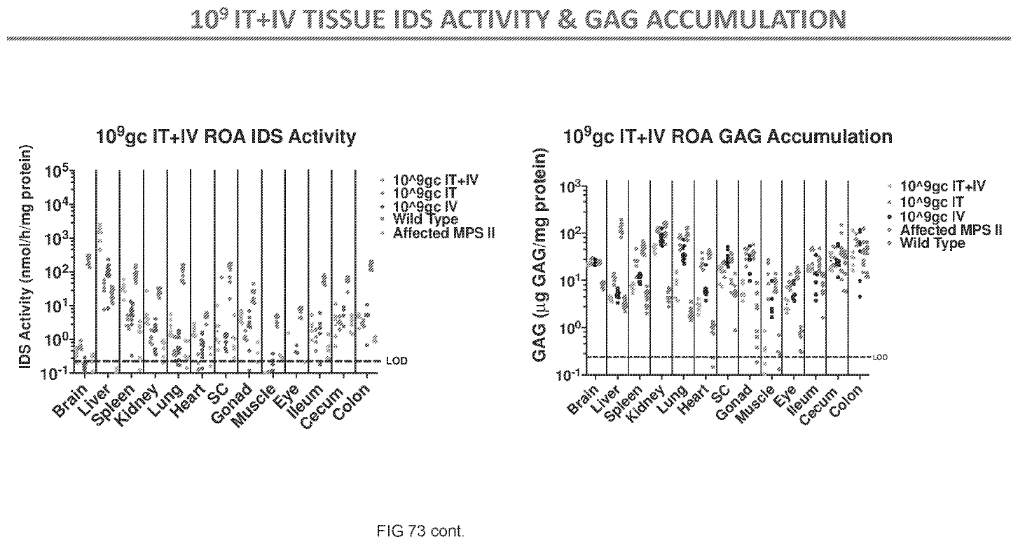

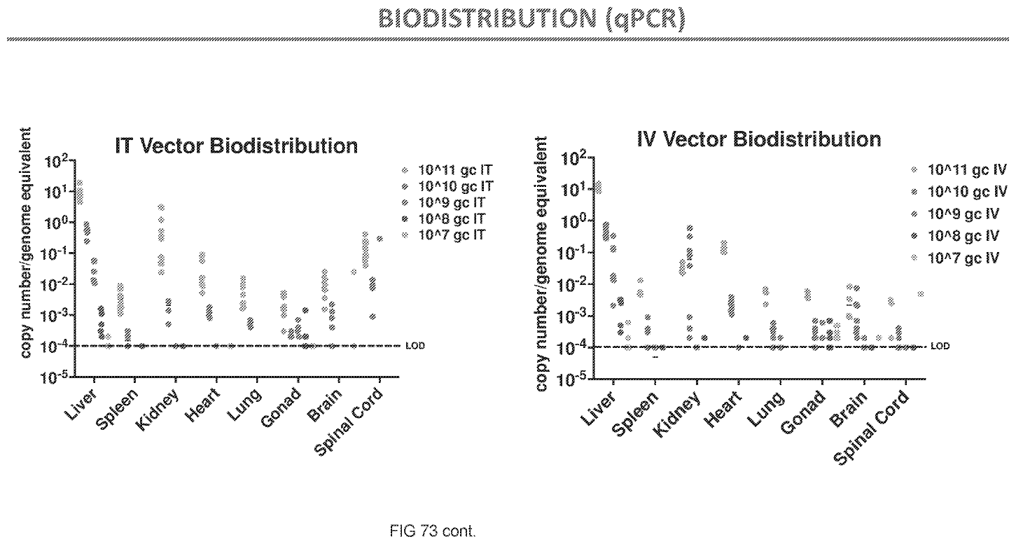

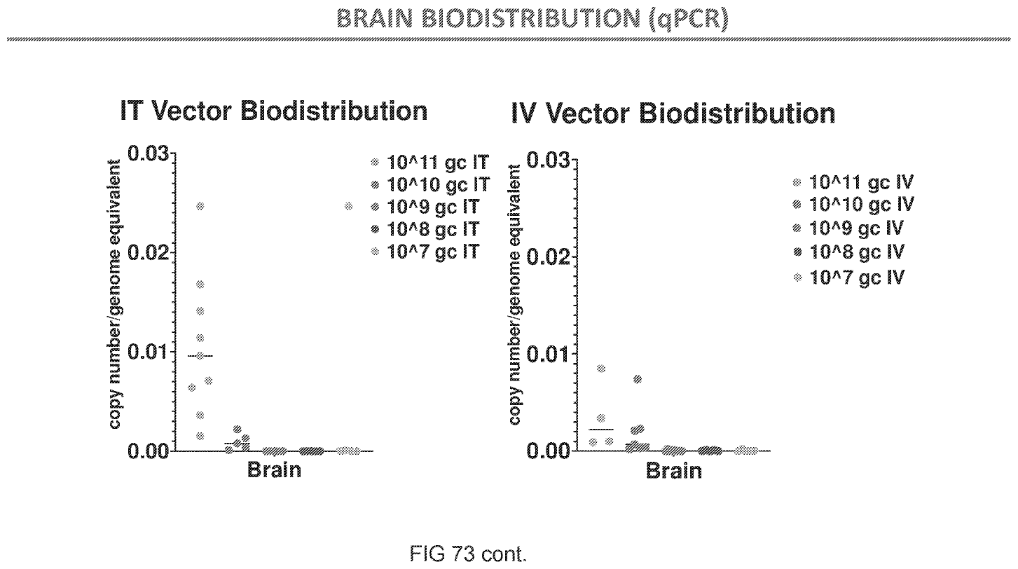

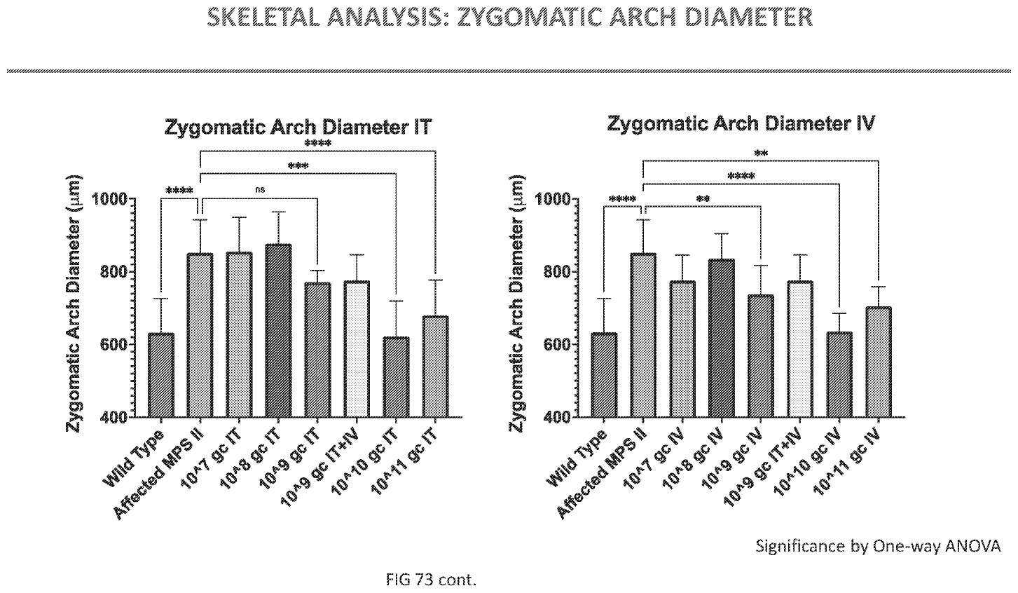

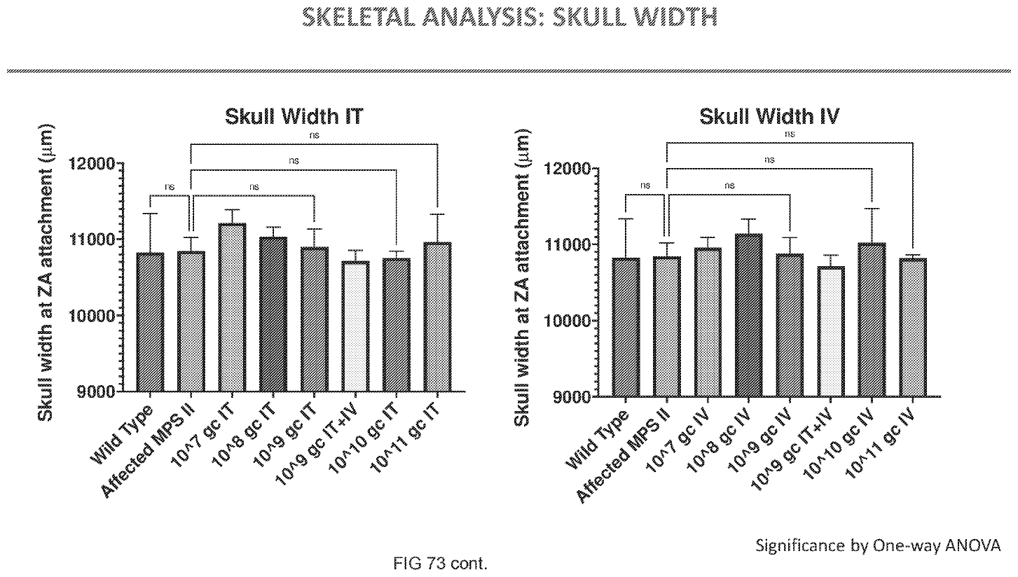

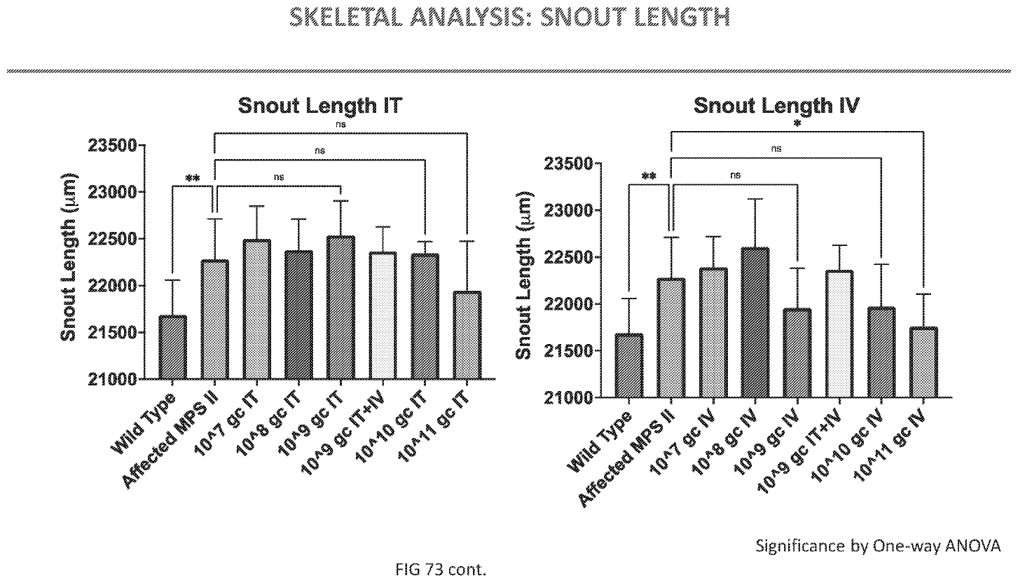

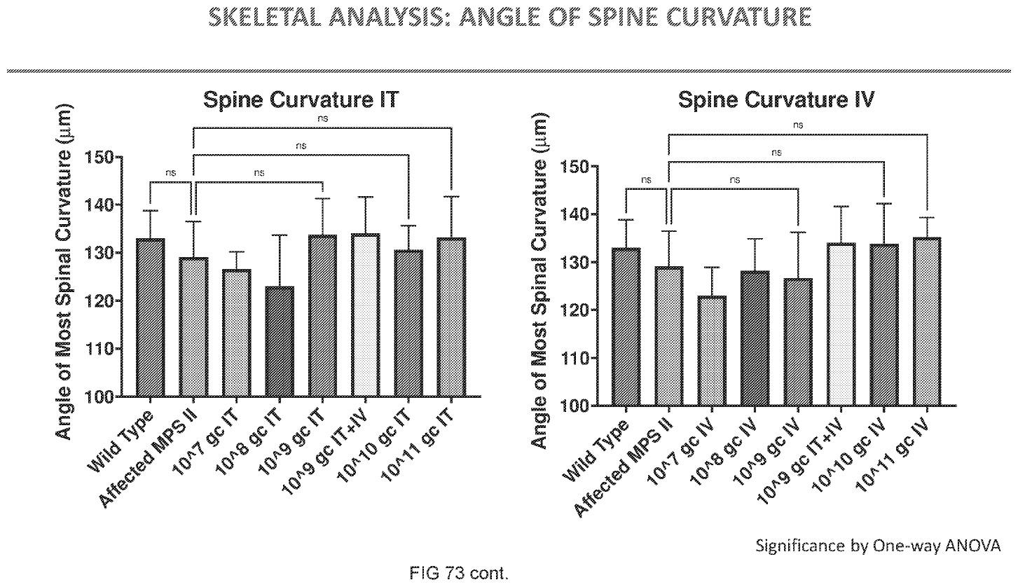

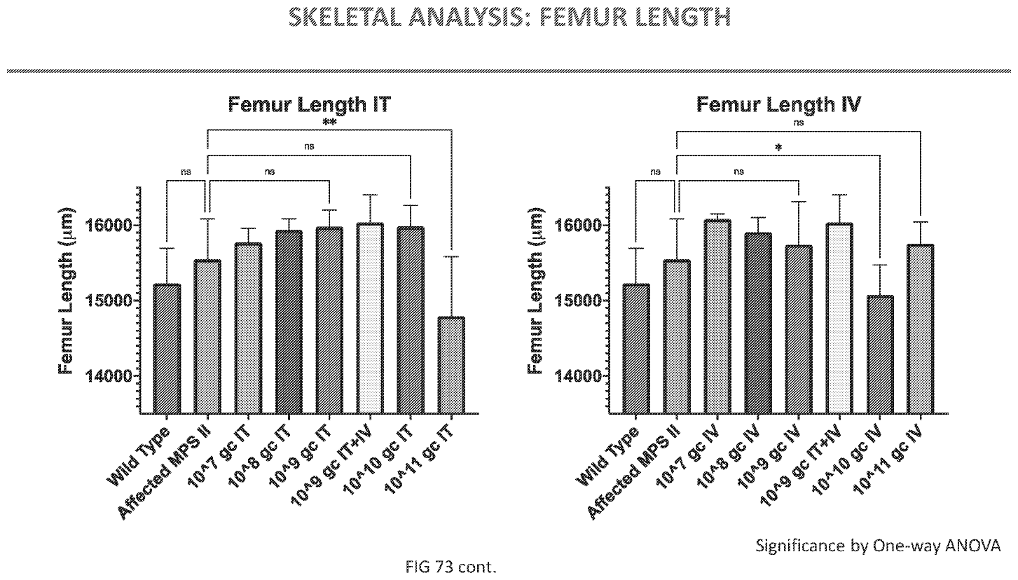

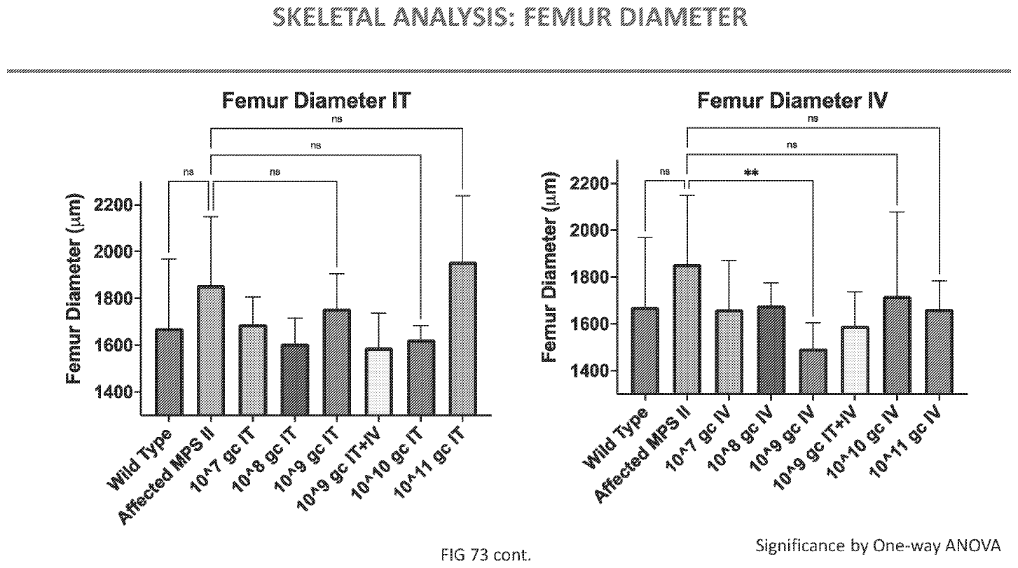

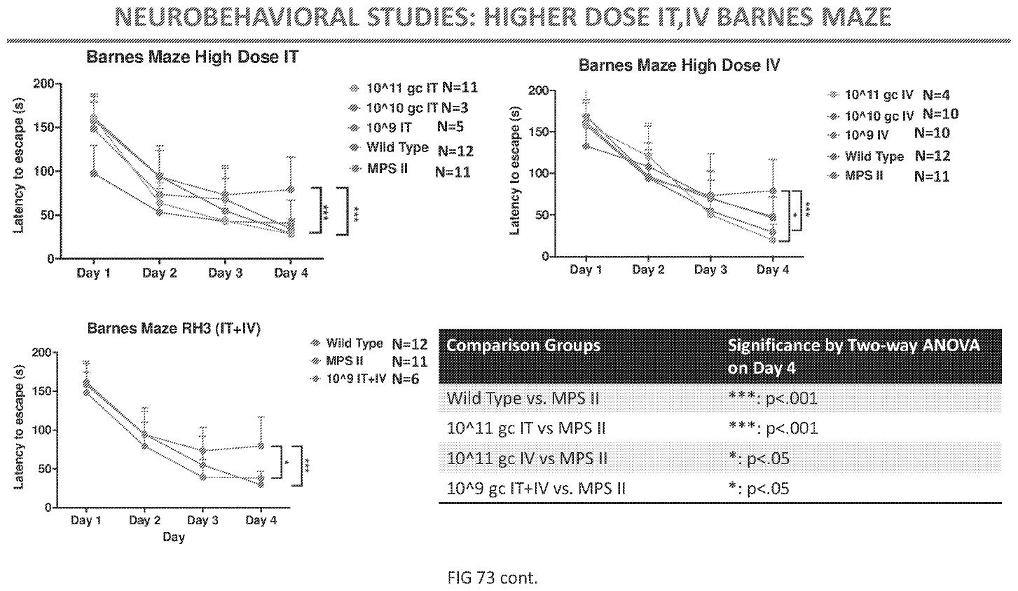

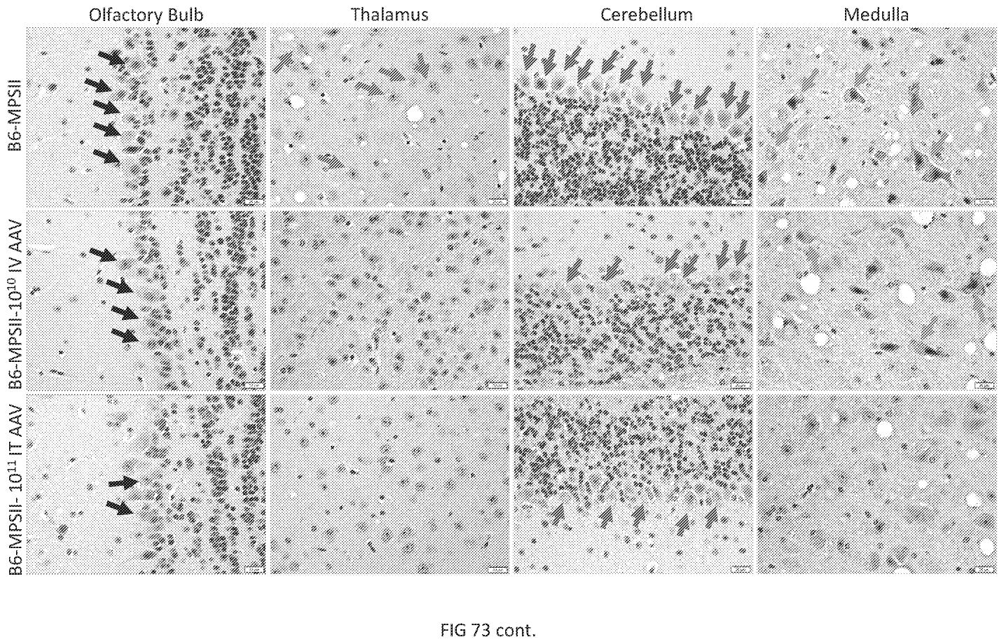

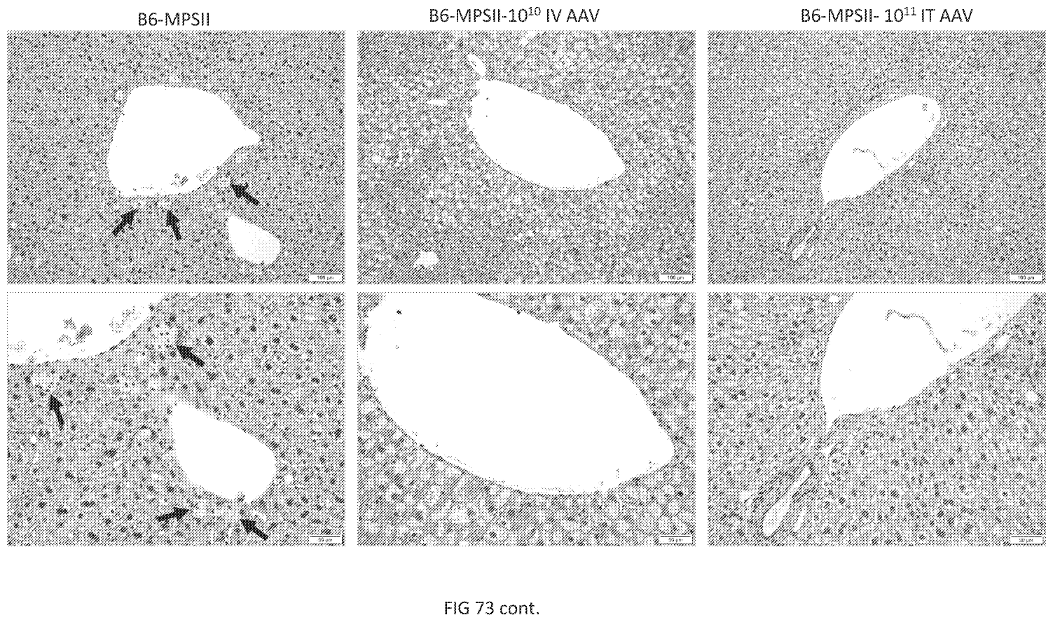

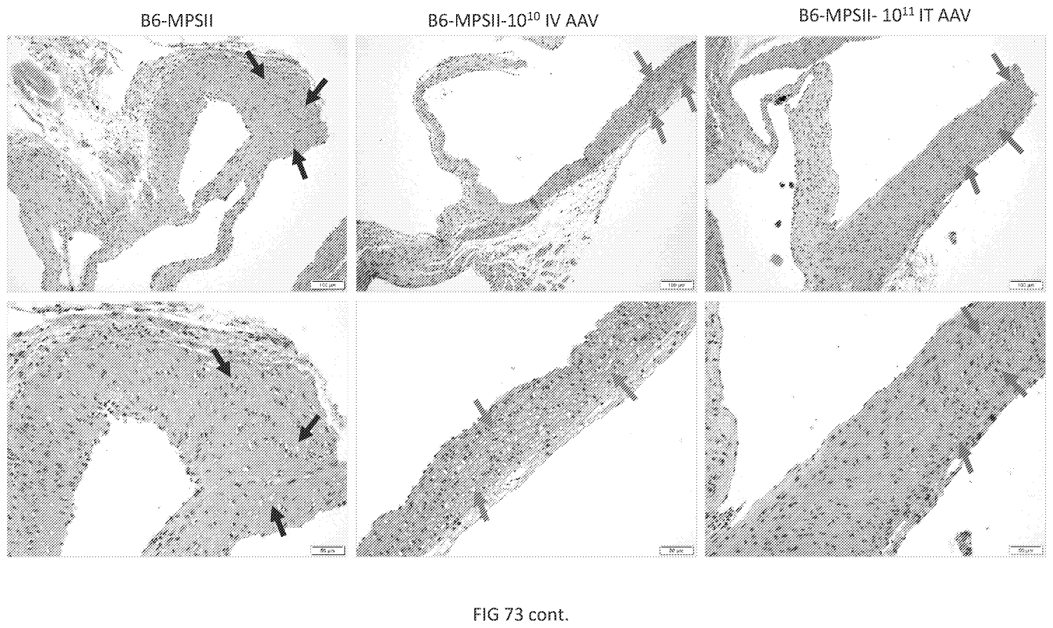

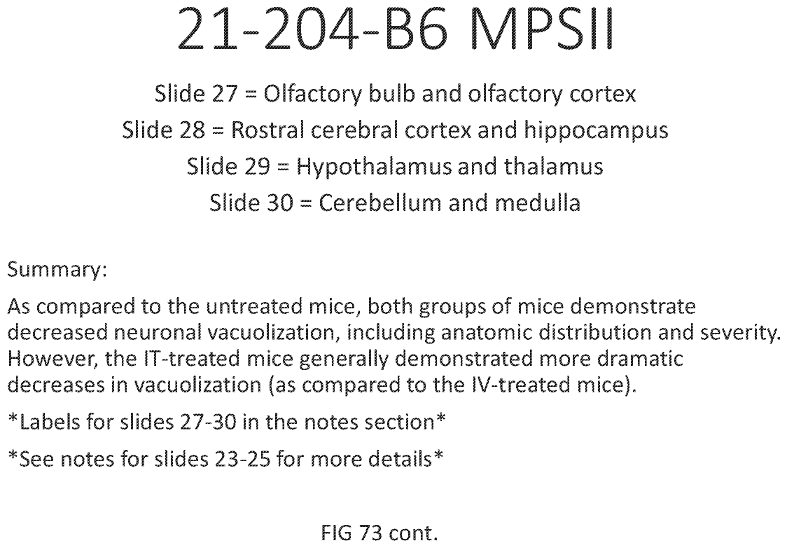

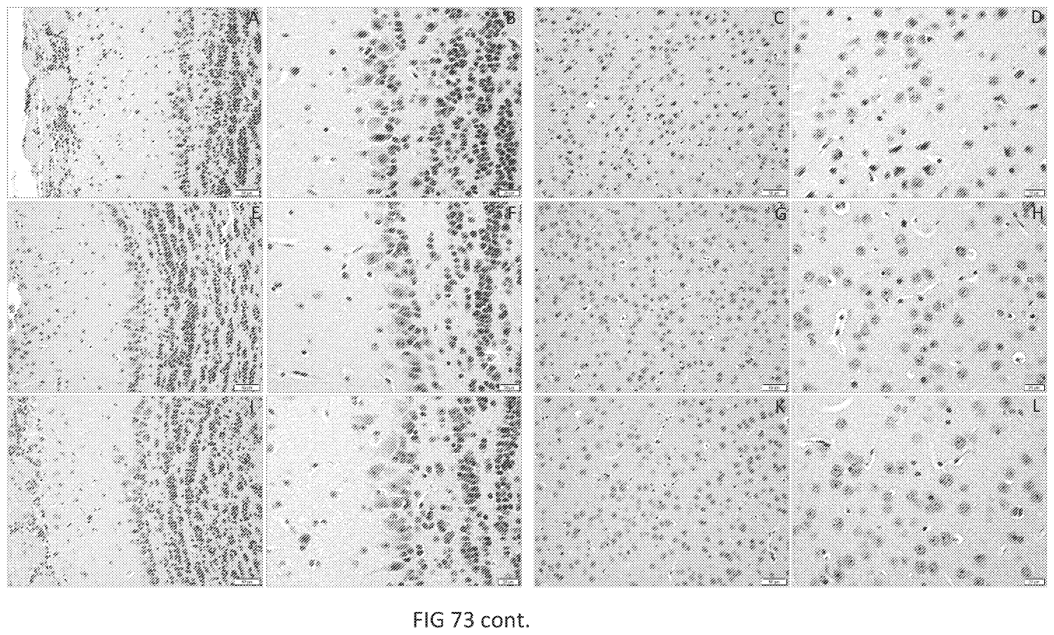

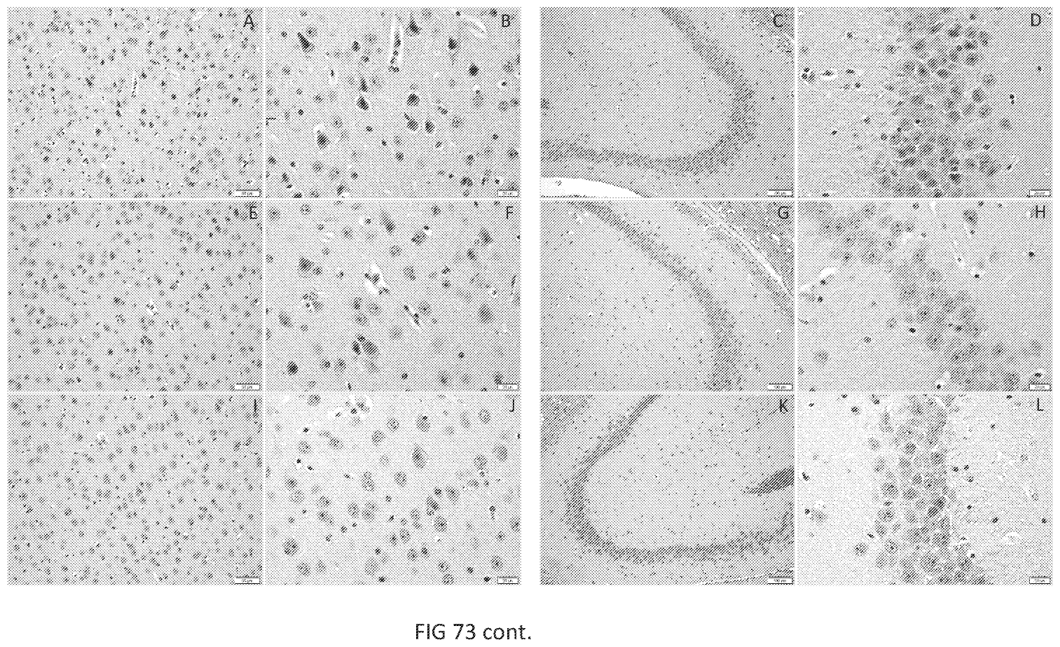

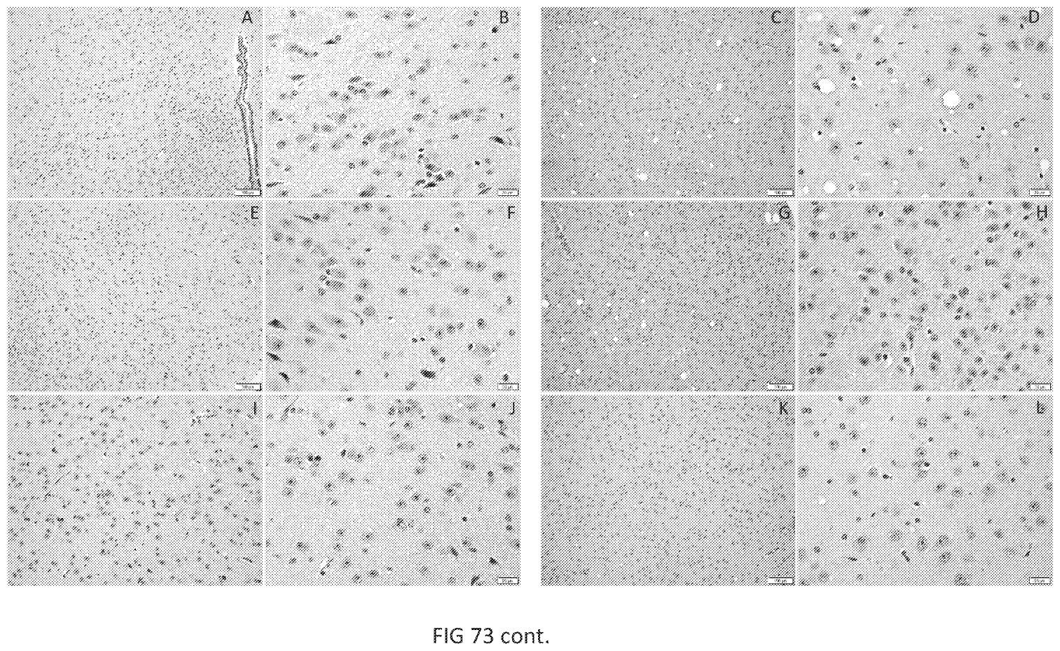

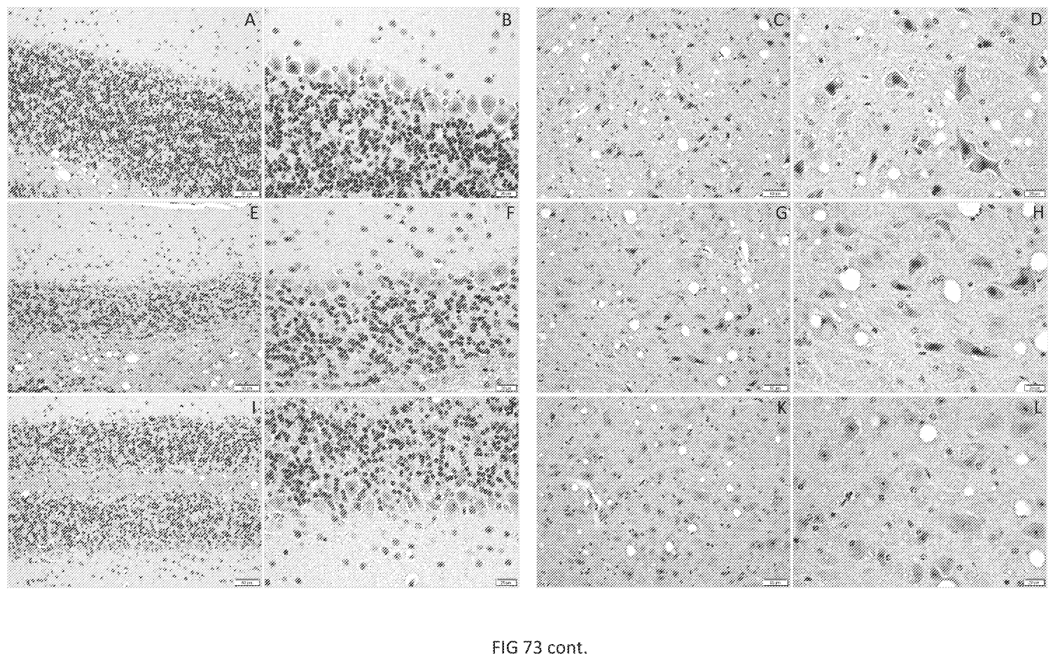

### See FIGS. 68-72.

Mucopolysaccharidosis type I (MPS I) is an autosomal recessive storage disease caused by deficiency of α-L-iduronidase (IDUA), resulting in accumulation of heparan and dermatan sulfate glycosaminoglycans (GAGs). Current treatments include allogeneic hematopoietic stem cell transplantation (HSCT) and enzyme replacement therapy (ERT). However, ERT is ineffective against CNS disease due to the inability of lysosomal enzymes to traverse the blood-brain barrier, and while there is neurologic benefit to HSCT, the level of correction is variable, and the procedure is associated with morbidity and mortality. Preclinical studies of IDUA gene therapy using AAV vectors have provided encouraging results for the treatment of MPS I.

In this study, the relative efficacy of intrathecal (IT), intravenous (IV), and combined routes (IT+IV) of AAV9-IDUA vector administration on cardiac, neurologic, and skeletal manifestations of disease were examines. AAV9-IDUA at a dose of 1×10¹⁰ vg was administered either IT, IV, or IT+IV to 2-month old MPS I mice. Control mice included normal heterozygotes and untreated MPS I mice. A gender related effect was observed for IDUA activity in plasma, with levels 100- to 1000-fold higher (in females and males respectively) than normal in all 3 groups. Cardiac valve function analysis by high resolution ultrasound biomicroscopy showed aortic insufficiency (AI) in most untreated MPS I mice, while the IT+IV group showed no aortic insufficiency, and the IT and IV groups had only 1 mouse in each group with AI. The ascending aortic diameter was normalized in the IV (all mice) and the IT/IT+IV groups (1 mouse had increased diameter), compared to untreated MPS I mice. IV, IT or IV+IT administration of AAV9-IDUA appeared to prevent the emergence of neurocognitive deficit exhibited in MPS I mice, as evaluated by Barnes maze and fear conditioning cognitive tests. Skeletal analysis of vector-treated mice by microCT showed normalization of skull width, zygomatic arch diameter, and kyphosis in male mice. Cross sectional moment of inertia was also normalized in both, male and female treated mice. Animals were euthanized at 6 months post-treatment, and tissues were harvested and assayed for IDUA activity and GAG levels. IDUA activity in brain and liver showed a gender-related effect, with a 10-fold higher level seen in males. Enzyme activities in the brain were highest after IT administration (10-fold higher than normal), with lowest levels seen after IV administration. Supraphysiological levels of IDUA in liver were seen for all three groups (100-1000-fold higher than normal in females and males, respectively). The three test groups yielded similar levels of enzyme activity in all other organs, and GAG levels were normalized or reduced in all groups. The results show that AAV9-IDUA vector, administered IV, IT or IV+IT resulted in high levels of enzyme activity in major organs, and all three treatments appeared to prevent neurocognitive deficit, cardiac valve dysfunction and skeletal dysplasias in MPS I mice as a model for genetic therapy of human MPS I.

### SUMMARY

Mice treated with AAV9-IDUA gene therapy had high and sustained levels of IDUA enzyme activity in plasma and tissues regardless of route of administration. Normalization of GAG levels were observed in all tissues studied in the treated groups. Also treated groups exhibited prevention of neurocognitive decline and prevention of aortic dysfunction in treated mice was also observed. Male mice treated with AAV9-IDUA treatment had improvement in femur diameter, and normalization of skull width, zygomatic arch, and prevention of kyphosis.

### REFERENCES

Al-Ghananeem et al., AAPS Pharm. Sci. Tech., 3:E5 (2002).
Aronovich et al., J. Gene Med., 9:403 (2007).
Aronovich et al., Mol. Ther., 17:1136 (2009).
Aronovich, Mol. Genet. Metab., 114:83 (2015).
Bagger et al., Eur. J. Pharm. Sci., 21:235-242 (2004b).
Bagger et al., Int. J. Pharm., 269:311-322 (2004a).
Baker et al., Exp. Brain Res., 63:461 (1986).
Balin et al., J. Comp. Neurol., 251:260-280 (1986).
Banks et al., J. Drug Target, 17:91-97 (2009).
Banks et al., J. Pharmacol. Exp. Ther., 309:469 (2004).
Banks, Biopolymers, 90:589 (2008).
Barakat et al., J. Pharm. Pharmacol., 58:63 (2006).
Barbier et al., Mol. Genet. Metab., 110:303 (2013).
Barnes, J. Comp. Physiol. Psychol., 93:74 (1979).
Baumgartner et al., Neuron., 58:639 (2008).
Belur et al., Mol. Ther., 22:S236 (2014).
Benedict et al., Neuroendocrinology, 86:136 (2007b).
Benedict et al., Neuropsychopharmacology, 32:239 (2007a).
Benedict et al., Psychoneuroendocrinology, 29:1326 (2004).
Bennett et al., Sci. Transl. Med., 4:120ra15 (2012).
Bjoraker et al., J. Dev. Behav. Ped., 27:290 (2006).
Blits et al., J. Neuros. Methods, 185:257 (2010).
Born et al., Nat. Neurosci., 5:514 (2002).
Boulton et al., Am. J. Physiol., 276:R818 (1999).
Boulton et al., Neuropathol. Appl. Neurobiol., 22:325 (1996).
Bradbury et al., Am. J. Physiol., 240:F329 (1981).
Bradbury et al., J. Physiol., 339:519 (1983).
Brady, Ann. Rev. Med., 57:283 (2006).
Broadwell et al., J. Comp. Neurol., 242:632 (1985).
Broekman et al., Neuroscience, 138:501 (2006).
Broomfiled et al., Mol. Genet. Metab., (2019).
Buck, In: Kandel E R, Schwartz J H, Jessell T M, editors. Principles of neural science. 4th edition. New York: McGraw-Hill Companies. pp. 625-652 (2000).
Buxer et al., J. Neurochem., 56:1012 (1991).
Cai et al., Sichuan Da Xue Xue Bao Yi Xue Ban, 39:438 (2008).
Campos et al., Meta. Brain Dis., 27:121 (2012).
Capsoni et al., Proc. Natl. Acad. Sci. USA, 99:12432 (2002).
Carare et al., Neuropathol. Appl. Neurobiol., 34:131 (2008).
Cauna, In: Proctor D F, Andersen I, editors. Amsterdam: Elsevier Biomedical Press. pp. 45-69 (1982).
Charlton et al., Int. J. Pharm., 338:94 (2007b).
Charlton et al., J. Drug Target, 15:370 (2007a).
Charlton et al., Pharm. Res., 25:1531 (2008).
Chen et al., J. Alzheimers Dis., 1:35 (1998).
Chen et al., J. Pharm. Sci., 95:1364 (2006).
Chen, Mol. Ther., 8:495 (2003).
Chow et al., J. Pharm. Sci., 88:754 (1999).
Chow et al., J. Pharm. Sci., 90:1729 (2001).
Clerico et al., In: Doty R L, editor. Handbook of olfaction and gustation. 2nd edition. New York: Marcel
Dekker, Inc. pp. 1-16 (2003). Campos et al., Meta. Brain Dis., 27:121 (2012).
Costantino et al., Int. J. Pharm., 337:1 (2007).
Cserr et al., Am. J. Physiol., 240:F319 (1981).
Cserr et al., Brain Pathol., 2:269 (1992).
Dahlin et al., Eur. J. Pharm. Sci., 14:75 (2001).
Danhof et al., American Association of Pharmaceutical Scientists Annual Meeting, Atlanta, GA (2008).
Danielyan et al., Eur. J. Cell. Biol., 88:315 (2009).
Davis et al., Clin. Pharmacokinet., 42:1107 (2003).
de Lorenzo, In: Wolstenholme G E W, Knight J, editors. Taste and smell in vertebrates. London: Churchill. pp. 151-175 (1970).
De Rosa et al., Proc. Natl. Acad. Sci. USA, 102:3811 (2005).
DeSesso, Qual. Assur., 2:213 (1993).
Desmanis et al., Ann. Neurol., 56:68 (2004).
Desnick et al., Ann. Rev. Genom. Hum. Genet., 13:307 (2012).
deSouza et al., Eur. Neuropsychopharmacol., 19:53 (2009).
Dhanda et al., Drug Del. Tech., 5:64 (2005).
Dhuria et al., J. Pharm. Sci., 98:2501 (2009b).
Dhuria et al., J. Pharmaceutical Sciences, 99:1654 (2010).
Dhuria et al., J. Pharmacol. Exp. Ther., 328:312 (2009a).
Diano et al., J. Clin. Invest., 118:26 (2008).
Dickson et al., Mol. Gen. Metab., 91:61 (2007).
Djupesland et al., Larvngoscope, 116:466 (2006).
Domes et al., Biol. Psychiatry, 61:731 (2007b).
Domes et al., Biol. Psychiatry, 62:1187 (2007a).
Dufes et al., Int. J. Pharm., 255:87 (2003).
Duque et al., Mol. Ther., 17:1187 (2009).
Einer-Jensen et al., Exp. Brain Res., 130:216 (2000b).
Einer-Jensen et al., Pharmacol. Toxicol., 87:276 (2000a).
Einer-Jensen et al., Reproduction, 129:9 (2005).
Ellinwood et al., Mol. Genet. Metab., 91:239 (2007).
Fehm et al., J. Clin. Endocrinol. Metab., 86:1144 (2001).
Field et al., J. Neurocytol., 32:317 (2003).
Fliedner et al., Endocrinology., 17:2088 (2006).
Foust et al., Nat. Biotech., 27:5 (2009).
Foust et al., Nat. Biotechnol., 27:59 (2009).
Fraldi et al., Hum. Mol. Genet., 16:2693 (2007).
Francis et al., Brain, 131:3311 (2008).
Fratantoni et al., Science, 162:570 (1968).
Frey et al., Drug Delivery, 4:87 (1997).
Frey I I, Drug Del. Tech., 2:46 (2002).
Fu et al., Mol. Ther., 19:1025 (2011).
Fuss et al., Eur. J. Neurosci., 22:2649 (2005).
Gao et al., Biomaterials, 27:3482 (2006).
Gao et al., Int. J. Pharm., 340:207 (2007a).
Gao et al., J. Control Release, 121:156 (2007b).
Gopinath et al., Current Ther. Res., 23:596 (1978).
Gozes et al., Curr. Alzheimer Res., 4:507 (2007).
Graff et al., Pharm. Res., 20:1225 (2003).
Graff et al., Pharm. Res., 22:235 (2005a).
Graff et al., Pharm. Res., 22:86 (2005b).
Gray, 15th revised edition (Classic Collectors edition). New York: Bounty Books (1978).
Grevers et al., Arch. Otorhinolaryngol., 244:55 (1987).
Groothuis et al., J. Cereb. Blood Flow Metab., 27:43 (2007).
Gross et al., J. Anat., 135:83 (1982).
Guastella et al., Biol. Psychiatry, 63:3 (2008).
Hadaczek et al., Mol. Ther., 14:69 (2006).
Hallschmid et al., Regul. Pept., 149:79 (2008).
Han et al., J. Mol. Med., 85:75 (2007).
Hanson et al., BMC Neurosci., 9:S5 (2008).
Hanson et al., Drug Del. Tech., 4:66 (2004).
Hanson et al., In: EPO, editor. Biopharm and HealthPartners Research Foundation (2008).
Hanson et al., San Diego, CA: Society for Neuroscience (2007).
Harrison et al., Learn Mem., 13:809 (2006).
Hartung et al., J. Am. Soc. Gene Ther., 9:866 (2004).
Hartung et al., Mol. Thera., 9:869 (2004).
Hashizume et al., Neuro. Oncol., 10:112 (2008).
Hatterer et al., Blood, 107:806 (2006).
Haurigot et al., J. Clin. Invest., 123:3254 (2013).
Herati et al., J. Gene Med., 10:972 (2008).
Hess et al., Exp. Neuro., 186:134 (2004).
Hinderer et al., Hum. Gene Ther., 27:906 (2016).
Hoogerbrugge et al., Lancet, 345:1398 (1995).
Horvat et al., Eur. J. Pharm. Biopharm., 72:252 (2009).
Huda et al., Mol. Ther. Methods Clin. Devel., 1:14032 (2014).
Hussar et al., Chem. Senses, 27:7 (2002).
Ilium, J. Control Release, 87:187 (2003).
Ilium, J. Pharm. Pharmacol., 56:3 (2004).
Itaya et al., Brain Res., 398:397 (1986).
Janson et al., Neurosurgery, 74:99 (2014).
Jansson et al., J. Drug Target, 10:379 (2002).
Jogani et al., Alzheimer Dis. Assoc. Disord., 22:116 (2008).
Johnston et al., Cerebrospinal Fluid Res., 1:2 (2004).
Kakkis et al., Mol. Gen. Met., 83:163 (2004).
Kandimalla et al., J. Pharm. Sci., 94:613 (2005b).
Kandimalla et al., Pharm. Res., 22:1121 (2005a).
Kaufmann et al., EMBO Mol. Med., 5:1642 (2013).
Kida et al., Neuropathol. Appl. Neurobiol., 19:480 (1993).
Kirsch et al., J. Neurosci., 25:11489 (2005).
Klein et al., J. Am. Soc. Gene Ther., 13:517 (2006).
Koos et al., Neuroreport, 16:1929 (2005).
Kosfeld et al., Nature, 435:673 (2005).
Kristensson et al., Acta Neuropathol (Berl), 19:145 (1971).
Krivit, Springer Seminars in Immunopathology, 26:119 (2004).
Kumar et al., Curr. Sci., 43:435 (1974).
Kumar et al., Int. J. Pharm., 358:285 (2008).
Li et al., Chin. J. Physiol., 48:7 (2005c).
Li et al., Glia, 52:245 (2005a).
Li et al., J. Neurocytol., 34:343 (2005b).
Lock et al., Hum. Gene Ther., 21:1259 (2010).
Loftus et al., Neuroscience, 139:1061 (2006).
Lum et al., J. Inherit. Metab. Dis., 40:455 (2017)
Luzzati et al., J. Mol. Biol., 343:199 (2004).
Mackay-Sim, In: Doty R L, editor. Handbook of olfaction and gustation. 2nd edition. New York: Marcel
Dekker, Inc. pp. 93-113 (2003).
Martinez et al., Neuroscience, 157:908 (2008).
McKinnis et al., J. Pediatr., 129:145 (1996).
Minn et al., J. Drug Target, 10:285 (2002).
Miragall et al., J. Comp. Neurol., 341:433 (1994).
Miyakawa et al., Hippocampus, 11:763 (2001).
Motas et al., JCI Insiqht. 1:e86696 (2016).
Muenzer, J. Pediatrics, 144:S27 (2004).
Munoz-Rojas et al., Am. J. Med. Gen., 146A:2538 (2008).
Nakai et al., J. Virol., 29:214 (2005).
Nathwani et al., New Engl. J. Med., 371:1994 (2014).
Neufeld and Muenzer, In A. L. B. C. R. Scriver, W. S. Sly, et al (ed.), McGraw Hill, NY, pg. 3421 (2001).
Nonaka et al., J. Pharmacol. Exp. Ther., 325:513 (2008).
Ohlfest et al., Blood, 105:2691 (2005).
Orchard et al., J. Pediatrics, 151:340 (2007).
Osborn et al., Mol. Ther., 19:450 (2011).
Ou et al., Mol. Genet. Metab., 111:116 (2014).
Owens et al., Diabet. Med., 20:886 (2003).
Pacak et al., Circ. Res., e3-e9 (2006).
Pan et al., Brain Res., 1188:241 (2008).
Pardridge, NeuroRx, 2:3 (2005).
Parker et al., Psychoneuroendocrinoloy, 30:924 (2005).
Pastores, Exp. Opin. Biol. Ther., 8:1003 (2008).
Paylor et al., Physiol. Behav., 73:781 (2001).
Pereira et al., Clin. Chim. Acta: Int. J. Clin. Chem., 387:75 (2008).
Perl et al., Lancet, 1:1028 (1987).
Peters et al., Bone Marrow Transpl., 31:229 (2003).
Polito et al., Am. J. Hum. Genet., 85:296 (2009).
Pollock et al., J. Anat., 191:337 (1997).
Raghavan et al., J. Laryngol. Otol., 114:456 (2000).
Rao et al., Ped. Res., 73:31 (2013).
Reger et al., J. Alzheimers Dis., 13:323 (2008a).
Reger et al., Neurobiol. Aginq, 27:451 (2006).
Reger et al., Neurology, 70:440 (2008b).
Rennels et al., Adv. Neurol., 52:431 (1990).
Rennels et al., Brain Res., 326:47 (1985).
Reolon et al., Brain Res., 1076:225 (2006).
Reolon et al., Cell. Mol. Neurobiol., 29:443 (2009).
Rimmele et al., J. Neurosci., 29:38 (2009).
Roberts et al., Pediatr. Res., 60:309 (2006).
Ross et al., J. Neuroimmunol., 151:66 (2004).
Ross et al., Neurosci. Lett., 439: 30 (2008).
Sabourdy et al., Orphanet. J. Rare Dis., 10:31 (2015).
Sakane et al., J. Pharm. Pharmacol., 46:378 (1994).
Sakane et al., J. Pharm. Pharmacol., 47:379 (1995).
Sarkar, Pharm. Res., 9:1 (1992).
Schaefer et al., J. Comp. Neurol., 444:221 (2002).
Scheibe et al., Arch. Otolaryngol. Head Neck Surg., 134:643 (2008).
Schley et al., J. Theor. Biol., 238:962 (2006).
Schulz et al., Endocrinology, 145:2696 (2004).
Schuster et al., Front Neuroanat., 8:42 (2014).
Scott et al., Am. J. Hum. Genet., 53:973 (1993).
Seeger et al., J. Neurosci., 24:10117 (2004).
Shimizu et al., Int. J. Obes. (Lond), 29:858 (2005).
Shipley, Brain Res. Bull., 15:129 (1985).
Skipor et al., Reprod. Biol., 3:143 (2003).
Steen et al., J. Alzheimers Dis., 7:63 (2005).
Souza et al. Studying learning and memory in animals: A look into cognitive function. in Cognitive Behavioral Therapy: Applications, Methods and Outcomes Vol. 4, Number 3, p. 37-70 (2012).
Stefanczyk-Krzymowska et al., Exp. Physiol., 85:801 (2000).
Takano et al., J. Histochem. Cytochem., 53:611 (2005).
Tanaka et al., Mol. Genet. Metab., 107:513 (2012).
Taylor et al., Biol. Blood Marrow Transpl., 25:e226 (2019).
Thorne et al., Brain Res., 692:278 (1995).
Thorne et al., Clin. Pharmacokinet., 40:907 (2001).
Thorne et al., Neuroscience, 127:481 (2004).
Thorne et al., Neuroscience, 152:785 (2008).
Thorne, R G. 2002. The nasal pathways for drug delivery to the central nervous system: Studies with protein tracers and therapeutics. Doctoral Dissertation, University of Minnesota.
Tkac et al., Maq. Res. In Med., 41:649 (1999).
Tkac et al., Maq. Res. In Med., 50:24 (2003).
Unger et al., J. Neuropath. Exp. Neuro., 52:460 (1993).
van den Berg et al., Eur. J. Pharm. Biopharm., 58:131 (2004b).
Van den Berg et al., J. Drug Target, 11:325 (2003).
van den Berg et al., J. Neurosci. Methods, 116:99 (2002).
van den Berg et al., Pharm. Res., 21:799 (2004a).
Van Diest et al., J. Anat., 128:293 (1979).
Vellodi et al., J. Inherit. Metab. Dis., 22:638 (1999).
Vulchanova et al., Mol. Pain, 6:31 (2010).
Vyas et al., AAPS PharmSciTech., 7:E1 (2006c).
Vyas et al., Crit. Rev. Ther. Drug Carrier Syst., 23:319 (2006b).
Vyas et al., J. Drug Target, 13:317 (2005).
Vyas et al., J. Pharm. Sci., 95:570 (2006a).
Walter et al., Arch. Histol. Cytol., 69:37 (2006a).
Walter et al., Neuropathol. Appl. Neurobiol., 32:388 (2006b).
Wang et al., Cancer Chemother. Pharmacol., 57:97 (2006a).
Wang et al., Eur. J. Pharm. Biopharm., 70:735 (2008).
Wang et al., Int. J. Pharm., 317:40 (2006b).
Wang et al., Int. J. Pharm., 341:20 (2007).
Watson et al., Gene Therapy, 16 Feb. 2006, doi:10.1038/sj.gt.3302735.
Weller et al., Neurol. Res., 25:611 (2003).
Westin et al., Eur. J. Pharm. Sci., 24:565 (2005).
Westin et al., Pharm. Res., 23:565 (2006).
Whitley et al., Am. J. Med. Genet., 46:209 (1993).
Williams et al., J. Comp. Neurol., 470:50 (2004).
Wioland et al., J. Histochem. Cytochem., 48:1215 (2000).
Wolf et al., Neurobio. Dis., 43:123 (2011).
Wolf et al., Neurology, 62:1503 (2004)
Xu et al., J. Clin. Invest., 118:272 (2008).
Yamada et al., Am. J. Physiol., 261:H1197 (1991).
Yang et al., J. Pharm. Sci., 94:1577 (2005).
Zhang et al., Acta Neuropathol. (Berl), 83:233 (1992).
Zhang et al., Acta Pharmacol. Sin., 25:522 (2004a).
Zhang et al., Int. J. Pharm., 275:85 (2004b).
Zhang et al., J. Druq Target, 14:281 (2006).
Zhao et al., Acta Pharmacol. Sin., 28:273 (2007).
Zhao et al., Chin. Med. Sci. J., 19:257 (2004).
Zheng et al., Mol. Genet. Metab., 79:233 (2003).
Ziegler and Shapiro, In J. Donders and S. Hunter (ed.), Cambridge University Press, p. 427 (2007).

All publications, patents and patent applications are incorporated herein by reference. While in the foregoing specification, this invention has been described in relation to certain preferred embodiments thereof, and many details have been set forth for purposes of illustration, it will be apparent to those skilled in the art that the invention is susceptible to additional embodiments and that certain of the details herein may be varied considerably without departing from the basic principles of the invention.

1. A method to prevent, inhibit the progression of, reduce the severity of, or treat cardiac, vascular
or skeletal dysfunction or defect(s) in a human having lysosomal storage disorder, comprising: administering to the human a first composition comprising an effective amount of a first recombinant adeno-associated virus (rAAV) vector comprising an open reading frame encoding a
first gene product and optionally a second composition comprising an amount of a second rAAV vector comprising an open reading frame encoding a second gene product, wherein if the first and second compositions are administered, they are administered via different routes.

2. The method of claim 1 wherein the amount reduces cardiac valve dysfunction, coronary artery abnormalities, myocardial abnormalities, conduction system abnormalities, or aortic root dilation.

3. The method of claim 1 wherein the amount decreases skeletal abnormalities.

4. The method of claim 1 wherein the amount increases skull width, cross-sectional moment of inertia (MOM) or bone stiffness.

5. The method of claim 1 wherein the first and the second compositions are administered.

6. The method of claim 5 wherein the first and the second gene products are the same.

7. The method of claim 5 wherein one of the routes is intravenous or intrathecal administration.

8. (canceled)

9. The method of claim 5 wherein the first and second compositions are concurrently administered.

10. The method of claim 5 wherein the first composition is administered before the second composition or the first composition is administered after the second composition.

11-12. (canceled)

13. The method of claim 1 wherein the mammal is not immunotolerized prior to administration of at least one of the rAAVs.

14. The method of claim 1 wherein the human is immunotolerized prior to administration of rAAV.

15. The method of claim 1 wherein at least one of the rAAV vectors is a rAAV1, rAAV3, rAAV4, rAAV5, rAAVrh10, or rAAV9 vector.

16. The method of claim 1 wherein the rAAV encodes alpha-L-iduronidase, iduronate-2-sulfatase, heparan sulfate sulfatase, N-acetyl-alpha-D-glucosaminidase, beta-hexosaminidase, alpha-galactosidase, betagalactosidase, beta-glucuronidase, or glucocerebrosidase

17. The method of claim 1 wherein the human is less than 5 years old.

18. The method of claim 1 wherein at least one of the rAAVs has an AAV9 or AAVrh10 capsid.

19. The method of claim 1 wherein the first and the second rAAVs have different capsids.

20. The method of claim 5 wherein the first composition is intravenously administered and the second composition is intrathecally administered

21-24. (canceled)

25. The method of claim 1 wherein the amount of rAAV administered is about 0.5×10¹⁰ to 4×10¹¹ genome copies (GC)/g brain tissue for intrathecal administration.

26. The method of claim 1 wherein the amount of rAAV administered is about 0.5×10¹¹ to 2×10¹² genome copies (GC)/kg of the human.


SEQUENCE LISTING (5 sequences)

SEQ ID NO: 1 - DNA, 20 bp, Artificial Sequence
  A synthetic sequence
gccaaaaattatggggacat

SEQ ID NO: 2 - DNA, 22 bp, Artificial Sequence
  A synthetic sequence
attccaacacactattgcaatg

SEQ ID NO: 3 - DNA, 27 bp, Artificial Sequence
  A synthetic sequence
atgaagccccttgagcatctgacttct

SEQ ID NO: 4 - DNA, 20 bp, Artificial Sequence
  A synthetic sequence
tcccttacctcgaccctttt

SEQ ID NO: 5 - DNA, 19 bp, Artificial Sequence
  A synthetic sequence
cacaaggtccatggattgc